(19) United States
(12) Patent Application Publication
(10) Pub. No.: US 2026/0061069 A1
(43) Pub. Date: Mar. 5, 2026

(54) SIZE-TUNABLE SYNTHETIC PARTICLES FOR IMMUNE CELL ACTIVATION

(71) Applicant: Slingshot Biosciences, Inc., Emeryville, CA (US)

(72) Inventors: Sunil THOMAS, San Bruno, CA (US); Harini KETHAR, Danville, CA (US); Daixuan ZHANG, Emeryville, CA (US); Juan ARMAS, Emeryville, CA (US); Subhanip BISWAS, Emeryville, CA (US); Kanwal PALLA, Oakland, CA (US); Martina DE GEUS, Berkeley, CA (US)

(21) Appl. No.: 19/307,843

(22) Filed: Aug. 22, 2025

Related U.S. Application Data
(63) Continuation of application No. PCT/US2024/018187, filed on Mar. 1, 2024.
(60) Provisional application No. 63/488,948, filed on Mar. 7, 2023.
(60) Provisional application No. 63/488,949, filed on Mar. 7, 2023.
(60) Provisional application No. 63/550,809, filed on Feb. 7, 2024.

(51) Int. Cl. A61K 47/69; A61K 47/64; A61P 37/04; C12N 5/00; C12N 5/0783

(52) U.S. Cl. CPC A61K 47/6927; A61K 47/6425; A61K 47/6903; A61P 37/04; C12N 5/0018; C12N 5/0636; C12N 2501/25; C12N 2501/51

(57) ABSTRACT

The present disclosure provides synthetic biomolecule presenting particles for immune cell activation.

### CROSS-REFERENCE TO RELATED APPLICATIONS

This application is a continuation of International Patent Application PCT/US2024/018187, filed Mar. 1, 2024, which claims the benefit of U.S. Provisional Application No. 63/488,949, filed Mar. 7, 2023, U.S. Provisional Application No. 63/488,948, filed Mar. 7, 2023, and U.S. Provisional Application No. 63/550,809, filed Feb. 7, 2024, each of which is herein incorporated by reference in its entirety.

### REFERENCE TO AN ELECTRONIC SEQUENCE LISTING

The contents of the electronic sequence listing (47357-716.601.xml; Size: 42,564 bytes; and Date of Creation: Feb. 29, 2024) is herein incorporated by reference in its entirety.

### BACKGROUND

Immunotherapy involving priming and expansion of immune cells, including T lymphocytes (T cells), is a promising treatment for cancer and other diseases (e.g., infectious diseases or autoimmune diseases). Current standards for in vitro T cell activation are magnetic microbeads containing αCD3 and αCD28 antibodies and having a subcellular sized diameter. Other methods to stimulate T cells in vitro include a plate-bound method where αCD3 and αCD28 antibodies are directly added to T cell culture and are washed off after 24 h of stimulation. Still other methods of T cells stimulation in vitro rely on autologous dendritic cells, virally infected B cells, and/or allogenic feeder cells cloned and injected with expanded T cells. However, these methods are inefficient, require billions of cells, and/or increase risk of undesirable immune reactions when the expanded T cells are administered to a patient. Accordingly, an improved method for immune cell activation is needed.

### SUMMARY

Aspects of the present disclosure relate to synthetic particles for immune cell activation. In some embodiments, the synthetic particle comprises (i) an antigen of the target immune cells; and/or (ii) immune co-stimulatory biomolecules that can activate 4-1BB receptor signaling, activate OX40 receptor signaling, and/or activate CD28 signaling.

Aspects of the present disclosure relate to a population of synthetic particles containing (i) an antigen of the target immune cells; and/or (ii) immune co-stimulatory biomolecules that can activate 4-1BB receptor signaling, activate OX40 receptor signaling, and/or activate CD28 signaling. In some embodiments, these functions may be carried out by different immune co-stimulatory biomolecules residing on different synthetic particles.

In some embodiments, the synthetic particles are biodegradable.

In some embodiments, the present disclosure provides methods of inducing an immune cell response (e.g., activation and/or expansion of the immune cells). In some embodiments, the present disclosure provides methods of treating diseases using the immune cells stimulated by such synthetic particles.

In some embodiments, the present disclosure provides methods of preparing such synthetic particles.

In one aspect, the disclosure provides synthetic particles comprising a matrix and at least one immune co-stimulatory biomolecule selected from the group consisting of: (i) a biomolecule that activates 4-1BB receptor signaling; (ii) a biomolecule that activates OX40 receptor signaling; (iii) a biomolecule that activates CD28 receptor signaling; and (iv) any combination thereof.

In one aspect, the disclosure provides synthetic particles comprising a matrix and at least one immune co-stimulatory biomolecule selected from the group consisting of a biomolecule that activates the signaling of CD3, a biomolecule that activates the signaling of CD28, a biomolecule that activates the signaling of ICOS (CD278), a biomolecule that activates the signaling of CD27 (TNFRSF7), a biomolecule that activates the signaling of CD40, a biomolecule that activates the signaling of CD40L, a biomolecule that activates the signaling of OX40 (CD134), a biomolecule that activates the signaling of 4-1BB (CD137), a biomolecule that activates the signaling of Toll-like receptor (TLR), a biomolecule that activates the signaling of HVEM (TNFSFR14 or CD270), a biomolecule that activates the signaling of LIGHT (TNFSF14, CD258), a biomolecule that activates the signaling of DR3 (TNFRSF25), a biomolecule that activates the signaling of GITR (CD357), a biomolecule that activates the signaling of CD30 (TNFRSF8), a biomolecule that activates the signaling of TIM1 (HAVCR1, KIM1), a biomolecule that activates the signaling of SLAM (CD150, SLAMF1), a biomolecule that activates the signaling of CD2 (LFA2, OX34), a biomolecule that activates the signaling of CD226 (DNAM1), and any combination thereof.

In one aspect, the disclosure provides synthetic biomolecule presenting particles comprising a matrix and at least one immune co-stimulatory biomolecule selected from the group consisting of: (i) a biomolecule that activates 4-1BB receptor signaling; (ii) a biomolecule that activates OX40 receptor signaling; (iii) a biomolecule that activates CD28 receptor signaling; and (iv) any combination thereof.

In one aspect, the disclosure provides synthetic biomolecule presenting particles comprising a matrix and at least one immune co-stimulatory biomolecule selected from the group consisting of a biomolecule that activates the signaling of CD3, a biomolecule that activates the signaling of CD28, a biomolecule that activates the signaling of ICOS (CD278), a biomolecule that activates the signaling of CD27 (TNFRSF7), a biomolecule that activates the signaling of CD40, a biomolecule that activates the signaling of CD40L, a biomolecule that activates the signaling of OX40 (CD134), a biomolecule that activates the signaling of 4-1BB (CD137), a biomolecule that activates the signaling of Toll-like receptor (TLR), a biomolecule that activates the signaling of HVEM (TNFSFR14 or CD270), a biomolecule that activates the signaling of LIGHT (TNFSF14, CD258), a biomolecule that activates the signaling of DR3 (TNFRSF25), a biomolecule that activates the signaling of GITR (CD357), a biomolecule that activates the signaling of CD30 (TNFRSF8), a biomolecule that activates the signaling of TIM1 (HAVCR1, KIM1), a biomolecule that activates the signaling of SLAM (CD150, SLAMF1), a biomolecule that activates the signaling of CD2 (LFA2, OX34), a biomolecule that activates the signaling of CD226 (DNAM1), and any combination thereof.

In one aspect, the disclosure provides synthetic particles comprising a matrix and at least one immune response biomolecule selected from the group consisting of: (i) a 4-1BB receptor; (ii) an OX40 receptor; (iii) a CD28 receptor; and (iv) any combination thereof.

In one aspect, the disclosure provides synthetic particles comprising a matrix and at least one immune response biomolecule selected from the group consisting of CD3, CD28, ICOS (CD278), CD27 (TNFRSF7), CD40, CD40L, OX40 (CD134), 4-1BB (CD137), Toll-like receptor (TLR), HVEM (TNFSFR14 or CD270), LIGHT (TNFSF14, CD258), DR3 (TNFRSF25), GITR (CD357), CD30 (TNFRSF8), TIM1 (HAVCR1, KIM1), SLAM (CD150, SLAMF1), CD2 (LFA2, OX34), CD226 (DNAM1), and any combination thereof.

In some embodiments, the immune response biomolecule is attached to the matrix via a linker. In some embodiments, the immune response biomolecule is non-covalently attached to the linker.

In some embodiments, the immune response biomolecule is tethered to an immune cell.

In some embodiments, the immune response biomolecule is attached to the matrix via the extracellular portion of the corresponding 4-1BB receptor, the OX40 receptor, and/or the CD28 receptor.

In some embodiments, the 4-1BB receptor is the human 4-1BB receptor. In some embodiments, the OX40 receptor is the human OX40 receptor. In some embodiments, the CD28 receptor is the human CD28 receptor.

In some embodiments, the 4-1BB receptor comprises a sequence at least 90%, at least 95%, at least 96%, at least 97%, at least 98%, at least 99%, or 100% identical to amino acids 24-255 of SEQ ID NO: 3. In some embodiments, the OX40 receptor comprises a sequence at least 90%, at least 95%, at least 96%, at least 97%, at least 98%, at least 99%, or 100% identical to amino acids 30-277 of SEQ ID NO: 4. In some embodiments, the CD28 receptor comprises a sequence at least 90%, at least 95%, at least 96%, at least 97%, at least 98%, at least 99%, or 100% identical to amino acids 28-220 of SEQ ID NO: 5.

In some embodiments, the extracellular portion of the 4-1BB receptor comprises a sequence at least 90%, at least 95%, at least 96%, at least 97%, at least 98%, at least 99%, or 100% identical to amino acids 24-159 of SEQ ID NO: 3. In some embodiments, the extracellular portion of the OX40 receptor comprises a sequence at least 90%, at least 95%, at least 96%, at least 97%, at least 98%, at least 99%, or 100% identical to amino acids 30-167 of SEQ ID NO: 4. In some embodiments, the extracellular portion of the CD28 receptor comprises a sequence at least 90%, at least 95%, at least 96%, at least 97%, at least 98%, at least 99%, or 100% identical to amino acids 28-137 of SEQ ID NO: 5.

In some embodiments, the synthetic particle of the disclosure comprises at least two of the biomolecules selected from the group consisting of (i)-(iii). In some embodiments, the synthetic particle of the disclosure comprises all three biomolecules selected from the group consisting of (i)-(iii).

In some embodiments, the synthetic particle comprises an antigen for an immune cell. In some embodiments, the antigen is CD19.

In some embodiments, the synthetic particle of the disclosure comprises a cell conjugated to the synthetic particle via the 4-1BB receptor, the OX40 receptor, and/or the CD28 receptor bound to the cell.

In one aspect, the disclosure provides a population of synthetic particles, said population comprising synthetic particles selected from the group consisting of: (a) synthetic particles comprising a biomolecule that activates 4-1BB receptor signaling; (b) synthetic particles comprising a biomolecule that activates OX40 receptor signaling; (c) synthetic particles comprising a biomolecule that activates CD28 receptor signaling; and (d) any combination thereof; wherein each of the synthetic particles comprises a polymer matrix.

In one aspect, the disclosure provides a population of synthetic particles, said population comprising synthetic particles selected from the group consisting of: (a) synthetic particles comprising a 4-1BB receptor immune response biomolecule; (b) synthetic particles comprising an OX40 receptor immune response biomolecule; (c) synthetic particles comprising a CD28 receptor immune response biomolecule; and (d) any combination thereof; wherein each of the synthetic particles comprises a polymer matrix.

In some embodiments, the immune response biomolecule is attached to the matrix via a linker. In some embodiments, the immune response biomolecule is non-covalently attached to the linker. In some embodiments, the immune response biomolecule is tethered to an immune cell. In some embodiments, the immune response biomolecule is attached to the matrix via the extracellular portion of the corresponding 4-1BB receptor; OX40 receptor, and/or the CD28 receptor. In some embodiments, the 4-1BB receptor is the human 4-1BB receptor. In some embodiments, the OX40 receptor is the human OX40 receptor. In some embodiments, the CD28 receptor is the human CD28 receptor. In some embodiments, the 4-1BB receptor comprises a sequence at least 90%, at least 95%, at least 96%, at least 97%, at least 98%, at least 99%, or 100% identical to amino acids 24-255 of SEQ ID NO: 3. In some embodiments, the OX40 receptor comprises a sequence at least 90%, at least 95%, at least 96%, at least 97%, at least 98%, at least 99%, or 100% identical to amino acids 30-277 of SEQ ID NO: 4. In some embodiments, the CD28 receptor comprises a sequence at least 90%, at least 95%, at least 96%, at least 97%, at least 98%, at least 99%, or 100% identical to amino acids 28-220 of SEQ ID NO: 5. In some embodiments, the extracellular portion of the 4-1BB receptor comprises a sequence at least 90%, at least 95%, at least 96%, at least 97%, at least 98%, at least 99%, or 100% identical to amino acids 24-159 of SEQ ID NO: 3. In some embodiments, the extracellular portion of the OX40 receptor comprises a sequence at least 90%, at least 95%, at least 96%, at least 97%, at least 98%, at least 99%, or 100% identical to amino acids 30-167 of SEQ ID NO: 4. In some embodiments, the extracellular portion of the CD28 receptor comprises a sequence at least 90%, at least 95%, at least 96%, at least 97%, at least 98%, at least 99%, or 100% identical to amino acids 28-137 of SEQ ID NO: 5.

In some embodiments, the population comprises (a). In some embodiments, the population comprises (b). In some embodiments, the population comprises (c). In some embodiments, the population comprises (a) and (b). In some embodiments, the population comprises (a) and (c). In some embodiments, the population comprises (b) and (c). In some embodiments, the population comprises (a), (b), and (c). In some embodiments, (a), (b), and (c) are distinct synthetic particles. In some embodiments, (a), (b) are the same synthetic particles that are distinct from (c). In some embodiments, (a), (c) are the same synthetic particles that are distinct from (b). In some embodiments, (b), (c) are the same synthetic particles that are distinct from (a). In some embodiments, (a), (b), and (c) are the same synthetic particles.

In some embodiments, at least one of the synthetic particles comprises a cell conjugated to the synthetic particle via a 4-1BB receptor, an OX40 receptor, and/or a CD28 receptor expressed by the cell.

In one aspect, the disclosure provides a population of synthetic particles comprising one or more synthetic particles of the disclosure.

In some embodiments, the population comprises one or more different subpopulations, each subpopulation comprises a different synthetic particle of the disclosure.

In some embodiments, the molar ratio of the biomolecule that activates 4-1BB receptor signaling to the biomolecule that activates OX40 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

In some embodiments, the molar ratio of the biomolecule that activates 4-1BB receptor signaling to the biomolecule that activates CD28 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

In some embodiments, the molar ratio of the biomolecule that activates OX40 receptor signaling to the biomolecule that activates CD28 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

In some embodiments, at least one synthetic particle comprises an antigen for an immune cell. In some embodiments, the antigen is CD19.

In one aspect, the disclosure provides a mixture of (i) cells and (ii) the population of synthetic particles of the disclosure. In some embodiments, the mixture is essentially free of feeder cells.

In one aspect, the disclosure provides cell-particle conjugates comprising a cell and the synthetic particle of the disclosure.

In one aspect, the disclosure provides cell-particle conjugates comprising a cell and the population of synthetic particles of the disclosure.

In one aspect, the disclosure provides cells conjugated to the synthetic particle of the disclosure.

In one aspect, the disclosure provides cells conjugated to the population of synthetic particles of the disclosure.

In some embodiments, the cell and the particle(s) are non-covalently conjugated.

In some embodiments, the cell expresses at least one of 4-1BB receptor, OX40 receptor, and CD28 receptor. In some embodiments, the cell expresses at least two of 4-1BB receptor, OX40 receptor, and CD28 receptor. In some embodiments, the cell expresses 4-1BB receptor, OX40 receptor, and CD28 receptor.

In some embodiments, the conjugation between the cell and the particle(s) comprises an interaction between at least one of (i) 4-1BB receptor and the biomolecule that activates 4-1BB receptor signaling, (ii) OX40 receptor and the biomolecule that activates OX40 receptor signaling, and (iii) CD28 receptor and the biomolecule that activates CD28 receptor signaling. In some embodiments, the conjugation comprises interactions between at least two of (i)-(iii). In some embodiments, the conjugation comprises interactions between all of (i)-(iii).

In some embodiments, the cell is an immune cell. In some embodiments, the immune cell is a T cell. In some embodiments, the immune cell is a cytotoxic T cell. In some embodiments, the immune cell is a CAR-T cell.

In some embodiments, the antigen binds to a chimeric antigen receptor (CAR) expressed by the immune cell.

In some embodiments, the biomolecule that activates 4-1BB receptor signaling comprises an anti-4-1BB receptor antibody or antigen binding fragment thereof, or comprises a 4-1BB ligand (4-1BBL) or a functional fragment thereof. In some embodiments, the 4-1BBL or the functional fragment thereof comprises an amino acid sequence at least 80%, at least 85%, at least 90%, at least 95%, at least 96%, at least 97%, at least 98%, at least 99%, or 100% identical to amino acids 80-244, or amino acids 50-254 of SEQ ID NO: 1. In some embodiments, the 4-1BBL or the functional fragment thereof is capable of activating the signaling of 4-1BB receptor expressed on a surface of an immune cell.

In some embodiments, the biomolecule that activates OX40 receptor signaling comprises an anti-OX40 receptor antibody or antigen binding fragment thereof, or comprises an OX40 ligand (OX40L) or a functional fragment thereof. In some embodiments, the OX40L or the functional fragment thereof comprises an amino acid sequence at least 80%, at least 85%, at least 90%, at least 95%, at least 96%, at least 97%, at least 98%, at least 99%, or 100% identical to amino acids 61-174, or amino acids 51-183 of SEQ ID NO: 2. In some embodiments, the OX40L or the functional fragment thereof is capable of activating the signaling of OX40 receptor expressed on a surface of an immune cell.

In some embodiments, the biomolecule that activates CD28 receptor signaling comprises an anti-CD28 antibody or antigen binding fragment thereof, a B7-1 (CD80) ligand or a functional fragment thereof, or a B7-2 (CD86) ligand or a functional fragment thereof. In some embodiments, the biomolecule that activates CD28 receptor signaling comprises an anti-CD28 antibody or antigen binding fragment thereof. In some embodiments, the anti-CD28 antibody is a mouse IgG1 monoclonal antibody (clone CD28.2) available from BioLegend®. In some embodiments, the biomolecule that activates CD28 receptor signaling binds CD28 receptor with a dissociation constant (Kd) of less than 10 μM, less than 1 μM, less than 100 nM, less than 10 nM, or less than 1 nM.

In some embodiments, the synthetic particle(s) further comprise a molecule selected from the group consisting of: a biologic; an antibody or an antigen-binding fragment thereof; an antibody drug conjugate; a protein; an enzyme; a peptide; a non-ribosomal peptide. In some embodiments, the synthetic particle(s) further comprise a molecule (e.g., antigen) selected from CD3; CD4; CD8; CD19; CD14; ccr7; CD45; CD45RA; CD27; CD16; CD56; CD127; CD25; CD38; HLA-DR; PD-1; CD28; CD183; CD185; CD57; IFN-gamma; CD20; TCR gamma/delta; TNF alpha; CD69; IL-2; Ki-67; CCR6; CD34; CD45RO; CD161; IgD; CD95; CD117; CD123; CD11c; IgM; CD39; FoxP3; CD10; CD40L; CD62L; CD194; CD314; IgG; TCR V alpha 7.2; CD11b; CD21; CD24; IL-4; Biotin; CCR10; CD31; CD44; CD138; CD294; NKp46; TCR V delta 2; TIGIT; CD1c; CD2; CD7; CD8a; CD15; CD32; CD103; CD107a; CD141; CD158; CD159c; IL-13; IL-21; KLRG1; TIM-3; CCR5; CD5; CD33; CD45.2; CD80; CD159a (NKG2a); CD244; CD272; CD278; CD337; Granzyme B; Ig Lambda Light Chain; IgA; IL-17A; Streptavidin; TCR V delta 1; CD1d; CD26; CD45R (B220); CD64; CD73; CD86; CD94; CD137; CD163; CD193; CTLA-4; CX3CR1; Fc epsilon R1 alpha; IL-22; Lag-3; MIP-1 beta; Perforin; TCR V gamma 9; CD1a; CD22; CD36; CD40; CD45R; CD66b; CD85j; CD160; CD172a; CD186; CD226; CD303; CLEC12A; CXCR4; Helios; IgKappaLight Chain; IgE; IgG1; IgG3; IL-5; IL-8; IL-21 R; KIR3dl05; KLRC1/2; Ly-6C; Ly-6G; MHC Class 11(1-A/I-E); MHC II; TCR alpha/beta; TCR beta; TCR V alpha 24; Akt (pS473); ALDH1A1; Annexin V; Bcl-2; c-Met; CCR7; cd16/32; cd41a; CD3 epsilon; CD8b; CD11b/c; CD16/CD32; CD23; CD29; CD43; CD45.1; CD48; CD49b; CD49d; CD66; CD68; CD71; CD85k; CD93; CD99; CD106; CD122; CD133; CD134; CD146; CD150; CD158b; CD158b1/b2; CD158e; CD166; CD169; CD184; CD200; CD200 R; CD235a; CD267; CD268; CD273; CD274; CD317; CD324; CD326; CD328; CD336; CD357; CD366; DDR2; eFluor 780 Fix Viability; EGF Receptor; EGFR (pY845); EOMES; EphA2; ERK1/2 (pT202/pY204); F4/80; FCRL5; Flt-3; FVS575V; FVS700; Granzyme A; HER2/ErbB2; Hes1; Hoechst (33342); ICAM-1; IFN-alpha; IgAQ1; IgAQ1/IgA2; IgA2; IgG2; IgG4; IL-1 RAcP; IL-6; IL-10; IL-12; IL-17; Integrin alpha 4 beta 7; Isotype Ctrl; KLRC1; KLRC2; Live/Dead Fix Aqua; Ly-6A/Ly-6E; Ly-6G/Ly-6C; Mannose Receptor; MDRT; Met (pY1234/pY1235); MMP-9; NGF Receptor p75; ORAI1; ORAI2; ORAI3; p53; P2RY12; PARP; cleaved; RT1B; S6 (pS235/pS236); STIM1; STIM2; TCR delta; TCR delta/gamma; TCR V alpha 24 J alpha 18; TCR V beta 11; TCR V gamma 1.1; TCR V gamma 2; TER-119; TIMP-3; TRAF3; TSLP Receptor; VDAC1; Vimentin; XCR1; and YAP1. In some embodiments, the molecule is an antigen for an immune cell.

In some embodiments, the synthetic particle(s) do not contain a CD3 binding molecule. In some embodiments, the synthetic particle(s) do not contain a CD8 binding molecule.

In some embodiments, the synthetic particle(s) further comprise at least one T cell stimulatory molecule and/or at least one T cell co-stimulatory molecule.

In some embodiments, the biomolecule is biotinylated.

In some embodiments, at least one surface of the matrix is functionalized. In some embodiments, the functionalized surface comprises a linker. In some embodiments, the functionalization comprises conjugating, coating, and/or embedding the linker to and/or within the matrix.

In some embodiments, the biomolecule is bound to the matrix via a linker. In some embodiments, the linker comprises streptavidin. In some embodiments, the biomolecule is non-covalently or covalently bound to the matrix.

In some embodiments, the matrix is a substantially spherical matrix.

In some embodiments, the matrix comprises a polymer material derived from one or more monomers. In some embodiments, the one or more monomers are selected from group consisting of: hydroxyethyl methacrylate, ethyl methacrylate, 2-hydroxyethyl methacrylate (HEMA), propylene glycol methacrylate, N-vinylpyrrolidone (NVP), methyl methacrylate, glycidyl methacrylate, glycerol methacrylate (GMA), glycol methacrylate, lactic acid, glycolic acid, poly(lactic-co-glycolic) acid (PLGA), ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzyl acrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, N-phenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, acrylamide, bisacrylamide, streptavidin-acrylamide, N-vinylcarbazole, 4-vinylpyridine, 2-vinylpyridine, agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitin, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan.

In some embodiments, the matrix is biodegradable.

In some embodiments, the one or more monomers comprise a monosaccharide, disaccharide, polysaccharide, peptide, protein, or protein domain. In some embodiments, the one or more monomers comprise a protein or protein domain comprising at least one non-natural amino acid. In some embodiments, the one or more monomers comprise a structural polysaccharide. In some embodiments, the one or more monomers are selected from the group consisting of agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitin, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan.

In some embodiments, the polymer material comprises poly(lactic-co-glycolic acid) (PLGA). In some embodiments, the PLGA has a composition of poly(lactic acid):poly(glycolic acid) of between about 90:10 and about 10:90.

In some embodiments, at least 50%, at least 60%, at least 70%, at least 80%, at least 90%, at least 95%, at least 98%, or at least 99% of the matrix is the polymer material derived from the one or more monomers.

In some embodiments, the synthetic particle(s) further comprise at least one fluorophore.

In some embodiments, the synthetic particle(s) have a (mean) diameter of between about 1 μm and about 40 μm, between about 10 μm and about 30 μm, between about 15 μm and about 25 μm, or about 20 μm.

In some embodiments, the synthetic particle(s) are hydrogel particles.

In some embodiments, the synthetic particle(s) have a (mean) porosity of about 5% to about 95% of a volume of the synthetic particle(s). In some embodiments, the synthetic particle(s) have a (mean) porosity of between about 80% and about 95% of the volume of the synthetic particle(s).

In some embodiments, the synthetic particle(s) comprise a plurality of micropores and a plurality of macropores within the matrix. In some embodiments, the mean diameter of the plurality of macropores is between about 200 nm and about 2 μm. In some embodiments, the synthetic particle comprises the plurality of macropores at a concentration of at least 2.25% v/v, at least 3.4% v/v, and/or at least 4.5% v/v. In some embodiments, the mean diameter of the plurality of micropores is between about 1 nm and about 20 nm. In some embodiments, between about 2 nm and about 4 nm. In some embodiments, the plurality of macropores comprise between about 2% and about 30% of a total number of pores of the synthetic particle, the total number of pores of the synthetic particle being a combination of the plurality of micropores and the plurality of macropores.

In some embodiments, the synthetic particle(s) exhibit a (mean) Young's modulus of between about 0.2 kPa and about 400 kPa.

In some embodiments, the biomolecule is located on a surface of the particle(s). In some embodiments, the surface of the particle is an internal surface or an external surface. In some embodiments, the internal surface is within the plurality of macropores.

In one aspect, the disclosure provides methods of inducing proliferation, expansion, and/or activation of immune cells in culture, comprising contacting or culturing the immune cells with the synthetic particle of the disclosure or the population of synthetic particles of the disclosure.

In one aspect, the disclosure provides methods of inducing an immune cell response, comprising contacting or culturing the immune cell with the synthetic particle of the disclosure or the population of synthetic particles of the disclosure. In some embodiments, the immune cell response includes activation and/or expansion of the immune cell. In some embodiments, the immune cell response is determined by (i) IL-2 secretion from the immune cell; (ii) CD25 expression from the immune cell; or (iii) CD69 expression from the immune cell. In some embodiments, the immune cell response is determined by interferon-gamma (IFNg) secretion from the immune cell. In some embodiments, the immune cell response from contacting the immune cell with the synthetic particle(s) is at least 50%, at least 100%, at least 2-fold, at least 3-fold, at least 5-fold, or at least 10-fold higher than the immune cell response from a control immune cell contacted with otherwise identical synthetic particle(s) lacking the biomolecule or macropores. In some embodiments, contacting comprises exposing the immune cells to the synthetic particles at a ratio of immune cell:synthetic particle of between about 1:0.5 and about 1:50, between about 1:1 and about 1:40, between about 1:2 and about 1:30, between about 1:5 and about 1:20, or about 1:10. In some embodiments, the contacting or culturing of the immune cell with the synthetic particle(s) lasts more than 8 hours.

In one aspect, the disclosure provides methods of treating a disease or disorder in a subject in need thereof, comprising administering the activated immune cells obtained by the method of the disclosure to the subject.

In one aspect, the disclosure provides methods of treating a disease or disorder in a subject in need thereof, comprising administering synthetic particle of the disclosure, the population of synthetic particles of the disclosure, the mixture of the disclosure, the cell-particle conjugate of any of the disclosure, or the cell of the disclosure, to the subject.

In some embodiments, the disease or disorder is a cancer, an autoimmune disease, or an infectious disease.

In one aspect, the disclosure provides methods of preparing the synthetic particle of the disclosure, comprising: preparing a precursor particle comprising the matrix and attaching the biomolecule to the precursor particle.

In some embodiments, the method comprises attaching the antigen for the immune cell to the precursor particle.

In one aspect, the disclosure provides methods of preparing or the population of synthetic particles of the disclosure, comprising: (i) preparing precursor particles comprising the matrix; (ii) attaching the biomolecules to the precursor particles. In some embodiments, step (ii) comprises attaching the two or more groups of biomolecule groups (i)-(iii) to separate precursor particles and then mixing the precursor particles. In some embodiments, the method comprises attaching the antigen for the immune cell to at least part of the precursor particle. In some embodiments, wherein preparing the precursor particle(s) comprises: mixing a base material with a porogen; forming microspheres from the mixture; thermally curing the microspheres; and washing the microspheres to remove the porogen, wherein the base material comprises a monomer and a linker.

In some embodiments, preparing the precursor particle(s) comprises: mixing a first phase comprising a monomer and porogens, with a second phase, wherein the first phase and the second phase are immiscible; polymerizing the first phase, thereby encapsulating or embedding porogens within the polymerized monomer; removing the porogens from the polymerized monomer to form the precursor particle(s). In some embodiments, the first phase is an aqueous phase and the second phase is a non-aqueous phase. In some embodiments, the first phase is a dispersed phase and the second phase is a continuous phase.

### BRIEF DESCRIPTION OF THE FIGURES

FIGS. 1A-1B illustrate the optical properties of disclosed hydrogel particles compared to polystyrene beads.

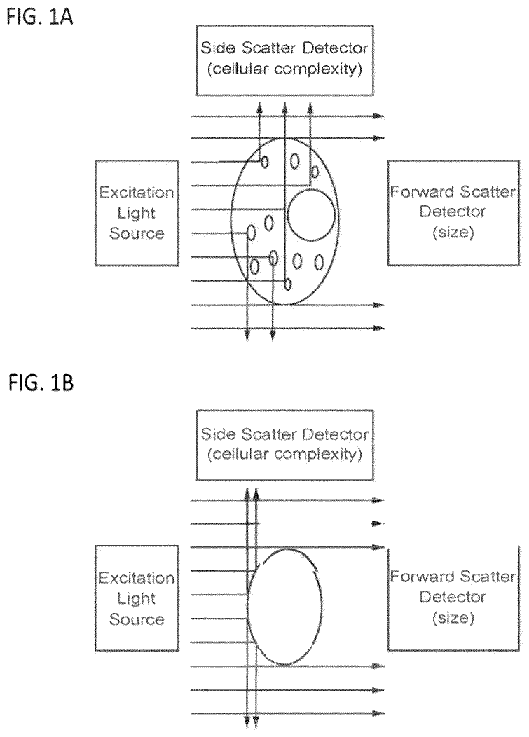

FIG. 2 depicts the process of producing labeled hydrogel particles of the disclosure, including hydrogels with attached biomolecules.

FIGS. 3A-3C provide brightfield and fluorescent images of labeled hydrogel particles of the disclosure.

FIG. 4 shows a scatter plot of a porous particle and a general step for manufacturing of porous particles.

FIG. 5 provides illustrations of porous particles formed from porogens at a range of concentrations (weight by volume) within the dispersed phase. As shown in FIG. 5, the porogen may be polyethylene glycol 8000 at concentrations of 2.25%, 3.4%, 4.5%, 6.3%, and 9% w/v. By visual observation, the porosity of the porous particles increases with increasing content of polyethylene glycol 8000 in the water phase formulations. Each image of the porogen concentrations can be evaluated in view of the 50 μm scale bar in the 9% porogen image. Increased porosity can be used as a factor for increase SSC optical match of particles. Porosity can also help replicate visual morphologies of target cells. Further conjugation of biomolecules on particles can provide additional functionality, including immune response activation functions.

FIG. 6 provides scatter plots of side scatter data and forward scatter data for porous particles formed by varying porogen concentrations (weight by volume) within the dispersed phase. From left to right, the porous particles comprise polyethylene glycol 8000 at concentrations of 2.25%, 3.4%, and 4.5% w/v. The side scatter of the porous particles measured by flow cytometry increases with increasing content of polyethylene glycol 8000 in the water phase formulations, while the forward scatter is largely unchanged.

FIG. 7 provides scatter plots of side scatter data and forward scatter data for porous particles comprising a constant concentration of porogen and nanoparticles. From left to right, the porous particles are formed from 9% polyethylene glycol with nanoparticles at concentrations (weight by volume) of 0%, 0.0825%, and at 0.165% w/v. The plots illustrate that the side scatter of a particle can be controlled independently of its porosity.

FIG. 8 provides scatter plots of optical scatter of porous particles conjugated with fluorescent dyes. Fluorophores or dyes can be conjugated to the porous particles, which can then be used to mimic a stained cell in the applications of image cytometry or histology.

FIG. 9 is a schematic of a degradable particle, according to embodiments of the present disclosure.

FIG. 10 is a schematic of a particle as a synthetic feeder cell, according to embodiments of the present disclosure.

FIG. 11 is a schematic of a particle as a synthetic biomolecule presenting particle, according to embodiments of the present disclosure.

FIGS. 12A-12B relate to particles as feeder cells, according to embodiments of the present disclosure.

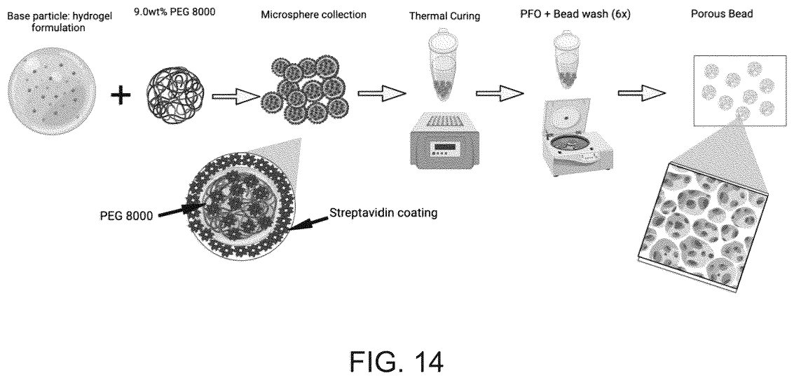

FIGS. 13A-13B relate to synthetic biomolecule presenting particles, according to embodiments of the present disclosure.

FIG. 14 depicts a method of generating porous particles by a microfluidic droplet process, the process including curing and purification before cell therapy application.

FIG. 15 is a microscopy image of porous particles formed using polyethylene glycol (PEG).

FIG. 16 depicts early-stage (24 hour incubation) activation of Jurkat samples incubated with either Dynabeads™ or porous particles, according to embodiments of the present disclosure. The porous particles of FIG. 16 are particles having pores formed during manufacturing using 9% w/v PEG as a porogen. FIG. 16 depicts an increased activation of Jurkat samples as indicated by upregulation of activation marker CD69 when compared with baseline Jurkats values and also when compared against cells activated by Dynabeads™.

FIG. 17 is a bar chart depicting early-stage T-cell activation (i.e., increase in Jurkat activation) when incubated with porous particles (pores formed by 9% PEG) and Dynabeads™ for 24 hours. As shown, T-cell activation is increased in porous particles samples, as shown by an increase in CD69.

FIG. 18 depicts a relative upregulation of early-stage T-cell activation marker CD69 in Jurkat samples incubated for 48 hours with porous particles (pores formed by 9% PEG) as compared to Dynabeads™. Activation during this prolonged incubation period represents a sustained activation.

FIG. 19 depicts a relative upregulation of late-stage T-cell activation marker CD25 in Jurkat samples incubated for 48 hours with porous particles (pores formed by 9% PEG) as compared to Dynabeads™. Activation during this prolonged incubation period represents a sustained activation.

FIG. 20 is a bar chart depicting a relative upregulation of late-stage T-cell activation marker CD25 in Jurkat samples incubated for 48 hours with porous particles (pores formed by 9% PEG) as compared to Dynabeads™.

FIG. 21 provides scatter plots of conjugation. 15 μm porous particles with 4.5% polyethylene glycol (MW 3550) and 0.4 mg/mL streptavidin acrylamide conjugated with EpCAM protein were stained with anti-EpCAM (Alexa Fluor® 405). Three different levels of EpCAM protein were evaluated (low, medium, high).

FIG. 22A is a chart showing the levels of secreted IFNg at 12-hour post co-culturing of indicated porous hydrogel particles with CAR-T Cells. FIG. 22B is a chart showing the levels of secreted IFNg at 24-hour post co-culturing of indicated porous hydrogel particles with CAR-T Cells.

FIG. 23A is a chart showing the levels of secreted IFNg at 24-hour post co-culturing of indicated porous hydrogel particles with CAR-T Cells. FIG. 23B is a chart showing the levels of secreted IFNg at 48-hour post co-culturing of indicated porous hydrogel particles with CAR-T Cells.

FIG. 24A is a chart showing the levels of secreted IFNg at 8-hour post co-culturing of indicated porous hydrogel particles with CAR-T Cells. FIG. 24B is a chart showing the levels of secreted IFNg at 24-hour post co-culturing of indicated porous hydrogel particles with CAR-T Cells.

FIG. 25 depicts poly(lactic-co-glycolic) acid (PLGA) particles providing significant upregulation of early-stage activation marker CD69 when compared to conventional stimulation methods (e.g., plate bound stimulation).

FIG. 26 is a histogram representation depicting a relative upregulation of late-stage T-cell activation marker CD25 in peripheral blood mononuclear cells five days after culture with PLGA particles functionalized with anti-CD3 and anti-CD28 antibodies. This upregulation, when compared to baseline activation of PBMCs, reflects sustained activation.

FIG. 27 is a schematic showing the activation of T cells by the synthetic beads, leading to expression of CD69 and CD25, and subsequent release of signaling molecules.

FIG. 28A is a bar chart showing the level of CD69 expressed by T cells stimulated with the indicated synthetic beads for 24-hour. FIG. 28B is a bar chart showing the level of CD25 expressed by T cells stimulated with the indicated synthetic beads for 96-hour.

FIG. 29 shows (upper) microscopy images of porous particles formed using the varying concentration of polyethylene glycol (PEG) (weight by volume) within the dispersed phase; (lower) scatter plots of side scatter data and forward scatter data for porous particles formed by varying concentrations of polyethylene glycol (PEG) (weight by volume) within the dispersed phase.

FIG. 30 shows flow cytometry dot plots of CD69 and CD25 expression by T cells following stimulation by the indicated particles. The numbers in the upper right quadrant of each graph shows % activated PBMC following incubation with each bead type.

### DETAILED DESCRIPTION

### Definitions

The indefinite articles “a” and “an” and the definite article “the” are intended to include both the singular and the plural, unless the context in which they are used clearly indicates otherwise.

“At least one” and “one or more” are used interchangeably to mean that the article may include one or more than one of the listed elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

The term “including all ranges and subranges therebetween” or equivalents, are used herein to denote the intention that disclosure of any range or series of possible values, inherently also discloses all ranges and subranges encompassed by the highest and lowest values disclosed. This term includes the entire range from highest to lowest disclosed values, as well as subranges from any two or more disclosed points. This term is also intended to disclose any subranges encompassed anywhere within the highest and lowest disclosed values, including between two points that are explicitly recited in the document, up to one decimal point. Thus, disclosure of values 0, 5, 10, 15, 20, including all ranges and subranges therebetween, should be interpreted as also encompassing a range from 0-20, a range from 0-5 or 5-15, as well as a range from 2-16, or 3.1 to 19.8, etc.

The term “Substantially similar,” as may be used herein, when used in reference to a property denotes at least 40% similar, at least 50% similar, at least 60% similar, at least 70% similar, at least 80% similar, at least 90% similar, at least 95% similar, at least 96% similar, at least 97% similar, at least 98% similar, or at least 99% similar to the property. For example, a particle having forward scatter property that is substantially similar to that of an target cell denotes that the forward scatter of the particle is at least 40% similar, at least 50% similar, at least 60% similar, at least 70% similar, at least 80% similar, at least 90% similar, at least 95% similar, at least 96% similar, at least 97% similar, at least 98% similar, or at least 99% similar to the forward scatter of the target cell.

As referred to herein, “porosity” may be used to refer to the percentage of void space within the particle. When porogens are used, the porosity is the percentage of void space within the particle after removal of the porogens. In such a case, the porosity may comprise a plurality of micropores and a plurality of macropores, as will be described below.

Unless otherwise indicated, it is to be understood that all numbers expressing quantities, ratios, and numerical properties of ingredients, reaction conditions, and so forth, used in the specification are contemplated to be able to be modified in all instances by the term “about”.

Unless otherwise indicated, it is to be understood that all numbers expressing quantities, ratios, and numerical properties of ingredients, reaction conditions, and so forth, used in the specification are contemplated to be able to be modified in all instances by the term “including all ranges and subranges therebetween”.

As may be used herein, the term “contacting” (i.e., contacting a cell e.g., a differentiable cell, with a compound or particle) is intended to include (but is not limited to) incubating the compound or particle and the cell together in vitro (e.g., adding the compound/particles to cells in culture). It is understood that the cells contacted with the defined medium can be further treated with a cell differentiation environment to stabilize the cells, or to differentiate the cells.

As may be used herein, the term “stabilize,” when used in reference to the differentiation state of a cell or culture of cells, indicates that the cells will continue to proliferate over multiple passages in culture, and preferably indefinitely in culture, where most, if not all, of the cells in the culture are of the same differentiation state. In addition, when the stabilized cells divide, the division typically yields cells of the same cell type or yields cells of the same differentiation state. A stabilized cell or cell population in general, does not further differentiate or de-differentiate if the cell culture conditions are not altered and the cells continue to be passaged and are not overgrown. In some embodiments, the cell that is stabilized is capable of proliferation in the stable state indefinitely, or for at least more than 2 passages. In a more specific embodiment, the cells are stable for more than 3 passages, more than 4 passages, more than 5 passages, more than 6 passages, more than 7 passages, more than 8 passages, more than 9 passages, more than 10 passages, more than 15 passages, more than 20 passages, more than 25 passages, or more than 30 passages. In some embodiments, the cell is stable for greater than approximately 1 month, 2 months, 3 months, 4 months, 5 months, 6 months, 7 months, 8 months, 9 months, 10 months, or 11 months of continuous passaging. In some embodiments, the cell is stable for greater than approximately 1 year of continuous passaging. In some embodiments, stem cells are maintained in culture in a pluripotent state by routine passage in the defined medium until it is desired that they be differentiated. As used herein, the term “proliferate” refers to an increase in the number cells in a cell culture.

Hence, as may be used herein, the term “growth environment” is an environment in which cells will proliferate in vitro. Features of the environment include the medium in which the cells are cultured, and a supporting structure (such as a substrate on a solid surface) if present.

As may be used herein, a “defined” medium refers to a biochemically defined formulation comprised solely of the biochemically defined constituents. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include particular compounds added to form the culture medium.

As may be used herein, the term “basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that supports the growth of primate embryonic stem cells in a substantially undifferentiated state can be employed. A “basal medium” as described herein also refers to the basal medium described in PCT/US2007/062755, filed Jun. 13, 2007, which is herein incorporated by reference in its entirety.

As may be used herein, the term “micropore” refers to porous structures within the particles that are naturally formed during the polymerization of the one or more monomer materials. The sizes of the micropores are typically small, with a diameter in the low nanometer range. The diameters of micropores rarely exceed 50 nm. In some embodiments, the mean diameter of the micropores is between about 1 nm and about 20 nm. In some embodiments, the mean diameter of the micropores is between about 2 nm and about 4 nm.

As may be used herein, the term “macropore” refers to porous structures within the particles that are larger than those naturally formed during the polymerization of the one or more monomer materials. Typically, macropores are created by first incorporating porogen material during the preparation of particles and then removing the porogen material from the particles. The diameters of macropores usually exceed 50 nm. In some embodiments, the mean diameter of the macropores is between about 200 nm and about 2 μm.

The term “antigen-binding fragment” refers to a polypeptide fragment that contains at least one complementarity-determining region (CDR) of an immunoglobulin heavy and/or light chain that binds to at least one epitope of the antigen of interest. Antigen-binding fragments include proteins that comprise a portion of a full length antibody, generally the antigen binding or variable region thereof, such as Fab, F(ab′)2, Fab′, Fv fragments, minibodies, diabodies, single domain antibody (dAb), single-chain variable fragments (scFv), and multispecific antibodies formed from antibody fragments.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is determined using the National Center for Biotechnology Information (NCBI)'s Basic Local Alignment Search Tool (BLAST®), available at blast.ncbi.nlm.nih.gov/Blast.cgi, version BLAST+2.13.0.

### Overview

Current methods used to activate and subsequently expand immune cells (e.g., T-cells) in vitro lead to cell exhaustion or require multi-step processes to remove activation agents from culture due to incompatibility with long-term cell survival. Accordingly, the present disclosure provides methods for improving the in vitro activation and/or expansion of immune cells.

In some embodiments, the present disclosure further relates to the use of the particles of the disclosure as synthetic biomolecule presenting particles.

In embodiments, in order to be used as a biomolecule presenting particle, the particles may be functionalized. After the particles are formed, a biomolecule (or other stimulating factor or marker) can be attached to a surface of the particles using binding chemistries based on the particle composition. These biomolecules may be selected based on particular cell surface markers of interest. These markers of interest may be one or more cell surface markers, or fragments thereof, for example, extracellular portions thereof in the case of transmembrane proteins. In some embodiments, the biomolecules may be antibodies or antigen-binding fragments thereof related to the particular cell surface marker of interest. In some embodiments, the biomolecules may be one or more cell surface markers, extracellular portions or ligand binding regions thereof.

In some embodiments, the biomolecules may be attached to the particle via a free amine, free carboxyl and/or free hydroxyl group present on the surface of the particle.

Functionalization of a particle with a cell surface molecule can also occur through a linker, such as by a streptavidin/biotin conjugate, a biotin/streptavidin conjugate, a streptavidin/biotin/streptavidin conjugate, and/or a biotin/streptavidin/biotin conjugate. For instance, when the particle comprises acrylamide, a streptavidin-biotin linkage can be exploited to attach particular biomolecules to the surface of the particles. Other known binding/linkage methods can be used without departing from the spirit of the present disclosure. In some embodiments, the linker comprises a polypeptide, a ligand, or an antibody. In some embodiments, the particle is capable of attaching to an immune response biomolecule via the linker. In some embodiments, the immune response biomolecule is located on the surface of a cell. In some embodiments, the cell may be attached to the particle via the linker.

In some embodiments, the disclosure provides compositions and methods for activating immune cells. In some embodiments, the disclosure provides functional synthetic cell mimics (e.g., synthetic particles) that can engage and activate immune cells (e.g., CAR-T cells). In some embodiments, the synthetic cell mimics are particles that contain (i) an antigen for the immune cells and/or (ii) at least one immune co-stimulatory biomolecule. In some embodiments, the at least one immune co-stimulatory biomolecule is selected from the group consisting of: (i) a biomolecule that activates 4-1BB receptor signaling; (ii) a biomolecule that activates OX40 receptor signaling; (iii) a biomolecule that activates CD28 receptor signaling; and (iv) any combination thereof. In some embodiments, the at least one immune co-stimulatory biomolecule is selected from the group consisting of: (i) 4-1BB ligand (4-1BBL) or a functional fragment thereof; (ii) OX40 ligand (OX40L) or a functional fragment thereof; (iii) a biomolecule that activates CD28 receptor signaling, and (iv) any combination thereof. In some embodiments, a population of the functional synthetic cell mimics (e.g., synthetic particles) contain the antigen for the immune cells and all these three types of immune co-stimulatory biomolecules (although, in some embodiments, different types of immune co-stimulatory biomolecules may be present on different synthetic cell mimics), and such a population of the functional synthetic cell mimics can better engage and activate the target immune cells than a control population of synthetic cell mimics that do not contain all these three types of immune co-stimulatory biomolecules. In some embodiments, such a population of the functional synthetic cell mimics may outperform live biological cells for engaging and activating immune cells. In some embodiments, the target immune cells are CAR-T cells and their activation leads to enhanced secretion of IFN-gamma (IFNg).

In some embodiments, the present disclosure teaches synthetic particles and/or populations of synthetic particles comprising one or more immune response biomolecules selected from the group consisting of (i) a 4-1BB receptor; (ii) an OX40 receptor; (iii) a CD28 receptor; and (iv) any combination thereof. In some embodiments, the immune response biomolecules are still tethered to an immune cell, such that the synthetic particle and the cell are connected via the immune response biomolecule. In some embodiments, the cell and the synthetic particle are connected via one or more linkers that interacts with the immune response biomolecules. In some embodiments, the linker interacts with the extracellular portion of the immune response biomolecule. For example, antibodies or ligands as linkers typically interact with the extracellular portions of receptors. In some embodiments, different linkers interact with different types of immune response biomolecules, such as a first linker that interacts with the 4-1BB receptor, a second linker that interacts with the OX40 receptor, and a third linker that interacts with the CD28 receptor. In some embodiments, the linker(s) are immune co-stimulatory biomolecules that activate the signaling of the immune response biomolecule(s). For example, in some embodiments, the cell and the synthetic particle are connected via one or more linkers selected from the group consisting of: (i) 4-1BB ligand (4-1BBL) or a functional fragment thereof; (ii) OX40 ligand (OX40L) or a functional fragment thereof; (iii) a biomolecule that activates CD28 receptor signaling (e.g., an anti-CD28 antibody), and (iv) any combination thereof.

In some embodiments, the configuration of the synthetic particles of the disclosure enhances the ability of the attached antigen and/or the immune co-stimulatory biomolecule to engage and activate target immune cells. In some embodiments, such enhancement is due to the presence of macropores in these synthetic particles which, without wishing to be bound to any particular theory, can result in (i) the provision of macropores as attachment sites for the antigen and/or the immune co-stimulatory biomolecule(s) to optimize their interactions with the immune cells; (ii) higher transportation rate of nutrients/water through pores; (iii) better absorption of water; (iv) maintenance of optimal ion nutrient gradient; and/or (v) maintenance of optimal osmotic pressure.

In some embodiments, the disclosure provides a mixture of cells with a population of the functional synthetic cell mimics (e.g., synthetic particles). In some embodiments, the disclosure provides cell-particle conjugates, which comprises cells conjugated to the functional synthetic cell mimic, or a population of the functional synthetic cell mimics. In some embodiments, the disclosure provides cells, wherein the cells are conjugated to the functional synthetic cell mimics. In some embodiments, the cells are non-covalently conjugated to the functional synthetic cell mimics.

Current methods used to activate and subsequently expand immune cells (e.g., T-cells) in vitro lead to cell exhaustion or require multi-step processes to remove activation agents from culture due to incompatibility with long-term cell survival. Accordingly, in some embodiments, the present disclosure provides methods for improving the in vitro activation and expansion of immune cells.

In some embodiments, the present disclosure relates to synthetic biomolecule presenting particles. Generally, the synthetic biomolecule presenting particles herein may be referred to as synthetic particles.

In some embodiments, the particles of the present disclosure comprise a polymer. The polymer may comprise a monomer selected from a group of monomers that includes lactic acid, glycolic acid, acrylic acid, 1-hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, a derivatized version thereof, or a combination thereof. In some embodiments, the polymer may be degradable. For instance, the polymer may be a polyester based on polylactide (PLA), polyglycolide (PGA), polycaprolactone, poly(lactic-co-glycolic) acid (PLGA), and their copolymers. Other biodegradable polymers may be used.

In embodiments, in order to be used as a biomolecule presenting particle, the particles may be functionalized. After the particles are formed, a biomolecule (or other stimulating factor or marker) can be attached to a surface of the particles using binding chemistries based on the particle composition (i.e., polymer). These biomolecules may be selected based on particular cell surface markers of interest. These markers of interest may be one or more cell surface markers, or fragments thereof, for example, extracellular portions thereof in the case of transmembrane proteins. For instance, the biomolecules may be antibodies related to the particular cell surface marker of interest. In some embodiments, the biomolecules may be one or more cell surface markers, extracellular portions or ligand binding regions thereof and may be attached to the particle via a free amine, free carboxyl and/or free hydroxyl group present on the surface of the particle. Functionalization of a particle with a cell surface molecule can also occur through a linker, such as by a streptavidin/biotin conjugate, a biotin/streptavidin conjugate, a streptavidin/biotin/streptavidin conjugate, and/or a biotin/streptavidin/biotin conjugate. Other known binding/linkage methods can be used without departing from the spirit of the present disclosure.

In embodiments, the particles of the present disclosure may be particles with enhanced porosity. Compared to non-porous particles, the alteration of pore size distribution allows more surface area per unit synthetic cell or more surface area per unit volume for advanced cell therapy. The porosity of the porous particle may be controlled by adjusting manufacturing parameters. For instance, the porosity may be controlled through the use of a porogen.

In some embodiments, cell therapy activation can be performed according to compositions and methods described herein. In embodiments, where the base polymer was formed using a porogen, each particle can be functionalized with biotinylated proteins for advanced cell activation. Exploiting the pore structure of this porous network permits improvements in cell response and cell proliferation. The introduction of pores into these particles, via e.g., PEG, could be used to improve biological response and lead to improved outcomes in biomedical, diagnostic, and therapeutics applications, especially cell activation therapy. It may be that the increased surface area to volume ratio introduced by these pores can enhance biological cell seeding by enabling more efficient mass transport such as cell signaling and cell cargo transport with enhanced liquid diffusion such as cell media to maximize cell proliferation. In any event, the generation of pores offers a number of advantages over non-porous structures. These include enhanced nutrient transport and higher surface to area to volume ratio.

In embodiments, the present disclosure relates to a PEG-based porous particle having a porosity that allows for higher protein/biomarker loading capacity, further allowing for improved cell stimulation. The fabricated particle allows for stronger bead-to-cell contact, and possible changes in Young's modulus, thereby affecting the quality of the stimulatory signal that the T cell receives and adhesion when compared to a monolayer slab (i.e., plate-bound activation method). Further, through utilizing streptavidin-biotin binding, biotinylated antigen and/or co-stimulatory biomolecules can be attached to streptavidin coated, porous particles, thereby allowing for engagement of immune receptors (e.g., chimeric antigen receptor) and/or immune response biomolecules (e.g., receptors) on T-cells.

In embodiments, the present disclosure relates to the use of a biodegradable polymer as a base polymer for the particles. The fabricated particle allows for stronger bead-to-cell contact, thereby affecting the quality of the stimulatory signal that the immune cell (e.g., T cell) receives and adhesion when compared to a monolayer slab (i.e., plate-bound activation method). In an example, utilizing streptavidin-biotin binding, biotinylated antigen and immune-costimulatory biomolecules are attached to streptavidin coated, PLGA particles, thereby allowing for engagement of immune receptors (e.g., chimeric antigen receptors) and immune response biomolecules (e.g., receptors) on the immune cells.

In embodiments, the particles of the present disclosure may comprise size-tunable microspheres fabricated via oil/water emulsion with PLGA and 1% polyvinyl alcohol. The microspheres are then coated with streptavidin and attached to biotinylated versions of biomolecules.

In embodiments, the base polymer of each particle can be selected based on a number of sites available for conjugation with a biomolecule. For instance, PLGA provides the ability to control numbers of conjugated biomolecules to the PLGA polymer backbone, thus allowing for control of cell activation. Further, the ability to control the composition of the polymer background allows for control of the rate of activation. For instance, in the case of PLGA, the ratio of PLA to PGA may be adjusted and/or the molecular weight of the polymer can be modified to enhance cellular activation.

Methods for tuning the properties of each particle are described herein. The ability to adjust a range of parameters including particle components and concentration of the same allows for the ability to tune a particle to mimic a wide range of cells, for example one of the cell types described herein.

As provided above, in some embodiments, the present disclosure provides individual particles each having one or more properties substantially similar to one or more properties of a target cell (e.g., size or elasticity).

The present disclosure is based in part on the unexpected discovery that one or more properties of a particle can be independently modulated by altering the composition of the particle, for example, by altering the amount of initial monomer (or co-monomer) in the composition, by altering the surface functionalization, by altering the amount of a polymerization initiator or by altering the amount of crosslinker. Furthermore, properties of particles can be tuned without having a substantial effect on density of the particle. This is a surprising and useful feature, as in some embodiments, particles that serve as surrogates for cells benefit from a minimal density in order to function appropriately.

In embodiments, a method for producing a particle is provided, wherein the particle has one or more properties substantially similar to the properties of one or more target cells. In some embodiments, the particle has pre-determined properties.

### Particles Comprising Immune Co-Stimulatory or Immune Response Biomolecule(s)

In some embodiments, the disclosure provides particles comprising one or more biomolecules. In some embodiments, the particles can present the biomolecules to cells, such as immune cells. In some embodiments, the particle comprises at least one immune co-stimulatory biomolecule. In some embodiments, the disclosure provides particles comprising one or more immune response biomolecules. In some embodiments, the immune response biomolecules are still tethered to an immune cell, such that the synthetic particle and the cell are connected via the immune response biomolecule. In some embodiments, the cell and the synthetic particle are connected via a linker that interacts with the immune response biomolecule.

In some embodiments, the particle comprising the biomolecule contains a covalent link between the particle and the biomolecule. In some embodiments, the linker biomolecule may further interact, covalently or non-covalently, with an immune response biomolecule bound to a cell (e.g., a biomolecule that activates 4-1BB receptor signaling on the particle interacting with a 4-1BB receptor bound to a cell). Accordingly, in some embodiments, the disclosure provides one or more particle(s) bound to a cell (e.g., a cell-particle conjugate) through the interaction between a linker on the particle and the counterpart immune response biomolecule bound to the cell.

In some embodiments, the linker comprises a protein, an antibody, a peptide, a small molecule, a fatty acid, a lipid, a saccharide, a macromolecule, a nucleic acid, an aptamer, and any combinations thereof. In some embodiments, the linker is a cleavable linker or a non-cleavable linker. In some embodiments, the linker is a linear linker or a branched linker. In some embodiments, the linker is a covalent linker or a non-covalent linker. In some embodiments, the linker is covalently linked on a first end (e.g., to the particle) and non-covalently linked on a second end (e.g., to the immune response biomolecule).

For example, in some embodiments, the cell and the synthetic particle are connected via a linker biomolecule selected from the group consisting of:(i) 4-1BB ligand (4-1BBL) or a functional fragment thereof; (ii) OX40 ligand (OX40L) or a functional fragment thereof; (iii) a biomolecule that activates CD28 receptor signaling (e.g., an anti-CD28 antibody), and (iv) any combination thereof. Thus, in some embodiments, the particle comprising the biomolecule (e.g., the immune response biomolecule) is formed by non-covalent interaction(s) between the particle and the biomolecule.

In some embodiments, the particle of the disclosure comprises one or more immune response biomolecules. In some embodiments, the one or more immune response biomolecules are selected from the group consisting of CD28, 4.1BB (CD137), OX40 (CD134), CD27 (TNFRSF7), GITR (CD357), CD30 (TNFRSF8), HVEM (CD270), LTβR (TNFRSF3), DR3 (TNFRSF25), ICOS (CD278), PD1 (CD279), CD226 (DNAM1), CRTAM (CD355), TIM1 (HAVCR1, KIM1), CD2 (LFA2, OX34), SLAM (CD150, SLAMF1), 2B4 (CD244, SLAMF4), Ly108 (NTBA, CD352, SLAMF6), CD84 (SLAMF5), Ly9 (CD229, SLAMF3), CRACC (CD319, BLAME), and any combination thereof. In some embodiments, the one or more immune response biomolecules are selected from the group consisting of CD3, CD28, ICOS (CD278), CD27 (TNFRSF7), CD40, CD40L, OX40 (CD134), 4-1BB (CD137), Toll-like receptor (TLR), HVEM (TNFSFR14 or CD270), LIGHT (TNFSF14, CD258), DR3 (TNFRSF25), GITR (CD357), CD30 (TNFRSF8), TIM1 (HAVCR1, KIM1), SLAM (CD150, SLAMF1), CD2 (LFA2, OX34), CD226 (DNAM1), and any combination thereof. In some embodiments, the one or more immune response biomolecules are selected from one or more immune response biomolecules listed in Table 9, and any combination thereof. In some embodiments, the particle of the disclosure comprises a combination of at least two, at least three, at least four, or at least five of the immune response biomolecules. In some embodiments, a population of the particles of the disclosure comprise a combination of at least two, at least three, at least four, or at least five of the immune response biomolecules. As noted in other portions of this disclosure, in some embodiments, the immune response biomolecule is connected to the synthetic particle via a linker. In some embodiments, the linker is an immune co-stimulatory biomolecule that activates the signaling of the corresponding immune response biomolecule. In some embodiments, the linker is attached to the extracellular portion of the immune response biomolecule.

In some embodiments, the particle of the disclosure comprises one or more immune co-stimulatory biomolecules. In some embodiments, the one or more immune co-stimulatory biomolecules are selected from the group consisting of a biomolecule that activates the signaling of CD3, a biomolecule that activates the signaling of CD28, a biomolecule that activates the signaling of ICOS (CD278), a biomolecule that activates the signaling of CD27 (TNFRSF7), a biomolecule that activates the signaling of CD40, a biomolecule that activates the signaling of CD40L, a biomolecule that activates the signaling of OX40 (CD134), a biomolecule that activates the signaling of 4-1BB (CD137), a biomolecule that activates the signaling of Toll-like receptor (TLR), a biomolecule that activates the signaling of HVEM (TNFSFR14 or CD270), a biomolecule that activates the signaling of LIGHT (TNFSF14, CD258), a biomolecule that activates the signaling of DR3 (TNFRSF25), a biomolecule that activates the signaling of GITR (CD357), a biomolecule that activates the signaling of CD30 (TNFRSF8), a biomolecule that activates the signaling of TIM1 (HAVCR1, KIM1), a biomolecule that activates the signaling of SLAM (CD150, SLAMF1), a biomolecule that activates the signaling of CD2 (LFA2, OX34), a biomolecule that activates the signaling of CD226 (DNAM1), and any combination thereof. In some embodiments, the one or more immune co-stimulatory biomolecules are selected from one or more immune co-stimulatory biomolecules listed in Table 9, and any combination thereof. In some embodiments, the particle of the disclosure comprises a combination of at least two, at least three, at least four, or at least five of the immune co-stimulatory biomolecules. In some embodiments, a population of the particles of the disclosure comprise a combination of at least two, at least three, at least four, or at least five of the immune co-stimulatory biomolecules. In some embodiments, the one or more immune co-stimulatory biomolecules comprise a ligand, a ligand mimic, an antibody, a peptide, an aptamer, a small molecule, or a combination thereof. In some embodiments, the immune co-stimulatory biomolecule binds the corresponding target biomolecule (e.g., an immune response biomolecule) with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules. In some embodiments, the one or more immune costimulatory biomolecules comprise an antibody that specifically binds the corresponding target biomolecule (e.g., an immune response biomolecule), or the antigen-binding fragment thereof. In some embodiments, the one or more immune costimulatory biomolecules comprise a ligand of the corresponding target biomolecule (e.g., an immune response biomolecule), or a functional fragment thereof.

Additional descriptions of immune response biomolecules and immune co-stimulatory biomolecules can be found, for example, in Chen and Flies, Nat Rev Immunol. 2013 April; 13(4):227-42; and Weinkove et al., Clin Transl Immunology. 2019 May 11; 8(5):e1049, the content of each of which is incorporated by reference herein in its entirety for all purposes.

TABLE 9
Non-limiting Examples of Immune Co-Stimulatory Biomolecules
and Corresponding Immune Response Biomolecule

| Non-limiting Examples of Immune Co-Stimulatory | |
|---|---|
| Biomolecule | Immune Response Biomolecule |
| CD3 agonist (e.g., anti-CD3 antibody or antigen | CD3 (formed by at least one of: |
| binding fragment thereof) | CD3 gamma (Uniprot ID: P09693; |
| | SEQ ID NO: 16); CD3 delta |
| | (Uniprot ID: P04234; SEQ ID NO: |
| | 17); CD3 epsilon (Uniprot ID: |
| | P07766; SEQ ID NO: 18); and/or |
| | CD3 zeta (Uniprot ID: P20963; |
| | SEQ ID NO: 19)) |
| CD80 (Uniprot ID: P33681; SEQ ID NO: 6) or | CD28 (Uniprot ID: P10747; SEQ |
| functional fragment thereof; CD86 (Uniprot ID: | ID NO: 5) |
| P42081; SEQ ID NO: 7) or functional fragment | |
| thereof; anti-CD28 antibody or antigen binding | |
| fragment thereof | |
| ICOS-L (CD275) (Uniprot ID: O75144) or functional | ICOS (CD278) (Uniprot ID: |
| fragment thereof (e.g., comprising SEQ ID NO: 13); | Q9Y6W8; SEQ ID NO: 12) |
| anti-ICOS antibody or antigen binding fragment | |
| thereof. | |
| CD70 (Uniprot ID: P32970) or functional fragment | CD27 (TNFRSF7) (Uniprot ID: |
| thereof (e.g., comprising SEQ ID NO: 15); anti-CD27 | P26842; SEQ ID NO: 14) |
| antibody or antigen binding fragment thereof. | |
| CD40L (CD154) (Uniprot ID: P29965) or functional | CD40 (Uniprot ID: P25942; SEQ |
| fragment thereof (e.g., comprising SEQ ID NO: 11); | ID NO: 8) |
| anti-CD40 antibody or antigen binding fragment | |
| thereof. | |
| CD40 (Uniprot ID: P25942) or functional fragment | CD40L (Uniprot ID: P29965; SEQ |
| thereof (e.g., comprising SEQ ID NO: 9); anti-CD40L | ID NO: 10) |
| antibody or antigen binding fragment thereof. | |
| OX40L (Uniprot ID: P23510; SEQ ID NO: 2) or | OX40 (CD134) (Uniprot ID: |
| functional fragment thereof; anti-OX40 antibody or | P43489; SEQ ID NO: 4) |
| antigen binding fragment thereof | |
| 4-1BBL (Uniprot ID: P41273; SEQ ID NO: 1) or | 4-1BB (CD137) (Uniprot ID: |
| functional fragment thereof; anti-4-1BB antibody or | Q07011; SEQ ID NO: 3) |
| antigen binding fragment thereof | |
| TLR agonist | Toll-like receptor (TLR) (e.g., |
| | TLR1 (Uniprot ID: Q15399); |
| | TLR2 (Uniprot ID: O60603); |
| | TLR3 (Uniprot ID: O15455); |
| | TLR4 (Uniprot ID: O00206); |
| | TLR5 (Uniprot ID: O60602); |
| | TLR6 (Uniprot ID: Q9Y2C9); |
| | TLR7 (Uniprot ID: Q9NYK1); |
| | TLR8 (Uniprot ID: Q9NR97); |
| | TLR9 (Uniprot ID: Q9NR96); or |
| | TLR10 (Uniprot ID: Q9BXR5)) |
| LIGHT (TNFSF14, CD258) (Uniprot ID: O43557) or | HVEM (TNFSFR14 or CD270) |
| functional fragment thereof (e.g., comprising SEQ ID | (Uniprot ID: Q92956; SEQ ID |
| NO: 21); anti-HVEM antibody or antigen binding | NO: 20) |
| fragment thereof | |
| HVEM (TNFSFR14 or CD270) (Uniprot ID: Q92956) | LIGHT (TNFSF14, CD258) |
| or functional fragment thereof (e.g., comprising SEQ | (Uniprot ID: O43557; SEQ ID |
| ID NO: 23); anti-LIGHT antibody or antigen binding | NO: 22) |
| fragment thereof | |
| TL1A (Uniprot ID: O95150) or functional fragment | DR3 (TNFRSF25) (Uniprot ID: |
| thereof (e.g., comprising SEQ ID NO: 25); anti-DR3 | Q93038; SEQ ID NO: 24) |
| antibody or antigen binding fragment thereof | |
| GITRL (Uniprot ID: Q9UNG2) or functional fragment | GITR (CD357) (Uniprot ID: |
| thereof (e.g., comprising SEQ ID NO: 27); anti-GITR | Q9Y5U5; SEQ ID NO: 26) |
| antibody or antigen binding fragment thereof | |
| CD30L (Uniprot ID: P32971) or functional fragment | CD30 (TNFRSF8) (Uniprot ID: |
| thereof (e.g., comprising SEQ ID NO: 29); anti-CD30 | P28908; SEQ ID NO: 28) |
| antibody or antigen binding fragment thereof | |
| TIM1 ligand or functional fragment thereof; TIM4 or | TIM1 (HAVCR1, KIM1) (Uniprot |
| functional fragment thereof; anti-TIM1 antibody or | ID: Q96D42; SEQ ID NO: 30) |
| antigen binding fragment thereof | |
| SLAM (Uniprot ID: Q13291) or functional fragment | SLAM (CD150, SLAMF1) |
| thereof (e.g., comprising SEQ ID NO: 32); anti-SLAM | (Uniprot ID: Q13291; SEQ ID |
| antibody or antigen binding fragment thereof | NO: 31) |
| CD48 (Uniprot ID: P09326) or functional fragment | CD2 (LFA2, OX34) (Uniprot ID: |
| thereof (e.g., comprising SEQ ID NO: 34); CD58 | P06729; SEQ ID NO: 33) |
| (Uniprot ID: P19256) or functional fragment thereof | |
| (e.g., comprising SEQ ID NO: 35); anti-CD2 antibody | |
| or antigen binding fragment thereof | |
| CD155 (Uniprot ID: P15151) or functional fragment | CD226 (DNAM1) (Uniprot ID: |
| thereof (e.g., comprising SEQ ID NO: 37); CD112 | Q15762; SEQ ID NO: 36) |
| (Uniprot ID: Q92692) or functional fragment thereof | |
| (e.g., comprising SEQ ID NO: 38); anti-CD226 | |
| antibody or antigen binding fragment thereof | |

In some embodiments, the immune co-stimulatory biomolecule binds to the corresponding target biomolecule (e.g., an immune response biomolecule tethered to a cell) with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules.

Generally, T cell activation is triggered by a peptide antigen bound to a major histocompatibility complex (MHC) molecule on the surface of an antigen presenting cell (APC), a T cell receptor/CD3 complex (TCR/CD3). While this is the primary signal in T cell activation, other receptor-ligand interactions between APC and T cells are also required for full activation. For example, TCR stimulation in the absence of other molecular interactions can induce an anergic state such that these cells cannot respond to a complete activation signal upon restimulation. Thus, optimal functionality may be conferred through the use of a second signaling molecule, such as a membrane bound protein or APC secretion product. For these membrane-bound proteins, such second interactions are usually adhesive in nature and enhance the contact between the two cells. Other signaling molecules (e.g., further activation signaling from APC to T cells) may also be relevant.

In some embodiments, the particles comprises one or more antibodies or antigen-binding fragments thereof that specifically bind to CD28, 4-1BB (CD137), OX40 (CD134), CD27 (TNFRSF7), GITR (CD357), CD30 (TNFRSF8), HVEM (CD270), LTβR (TNFRSF3), DR3 (TNFRSF25)), ICOS (CD278), PD1 (CD279), CD226 (DNAM1), CRTAM (CD355), TIM1 (HAVCR1, KIM1), CD2 (LFA2, OX34), SLAM (CD150, SLAMF1), 2B4 (CD244, SLAMF4), Ly108 (NTBA, CD352), SLAMF6), CD84 (SLAMF5), Ly9 (CD229, SLAMF3), and/or CRACC (CD319, BLAME).

In some embodiments, the particle comprises a T cell activation molecule selected from an anti-CD3 antibody or an antigen-binding fragment thereof, an anti-macrophage scavenger receptor (MSR1) antibody or an antigen-binding fragment thereof, an anti-T cell receptor (TCR) antibody or an antigen-binding fragment thereof, an anti-CD2 antibody or an antibody thereof, antigen-binding fragments, anti-CD47 antibodies or antigen-binding fragments thereof, major histocompatibility complex (MHC) molecules loaded with MHC peptides or multimers thereof, and MHC-immunoglobulin (Ig) conjugates or multimers thereof, and any combination thereof.

In some embodiments, the particle comprises a CD3 and a CD28 biomolecule or fragment thereof. In some embodiments, the particle comprises an anti-CD3 and an anti-CD28 antibody or antigen-binding fragment thereof.

In some embodiments, the particle comprises one or more molecules that can stimulate T cell expansion and/or activation. In some embodiments, the molecule that can stimulate T cell expansion and/or activation is a polypeptide or fragment thereof. In some embodiments, the polypeptide or fragment thereof that can stimulate T cell expansion and/or activation is a peptide antigen. In some embodiments, the molecule that can stimulate T cell expansion and/or activation is a component of an MHC molecule. In some embodiments, the molecule that can stimulate T cell expansion and/or activation is a component of a T cell receptor/CD3 complex. In some embodiments, the molecule that can stimulate T cell expansion and/or activation is an antibody that specifically binds a component of a T cell receptor/CD3 complex. In some embodiments, the particle of the present disclosure comprises an antibody or antigen-binding fragment therefore that specifically binds to CD3.

In some embodiments, the particle of the present disclosure comprises one or more T cell activation molecules and one or more immune response biomolecules. In some embodiments, the particle of the present disclosure comprises one or more antibodies or antigen-binding fragments thereof that specifically bind T cell activation molecules and one or more immune response biomolecules. In some embodiments, the particle of the present disclosure comprises a T cell activation molecule of CD3 and an immune response biomolecule selected from CD28, ICOS, CD27, CD40, and CD137 (or antibodies targeting said activation/immune response biomolecules).

In some embodiments, the particle of the present disclosure comprises one or more T cell activation molecules and one or more immune co-stimulatory biomolecules. In some embodiments, the particle of the present disclosure comprises one or more antibodies or antigen-binding fragments thereof that specifically bind T cell activation molecules and one or more immune response biomolecules. In some embodiments, the particle of the present disclosure comprises one or more antibodies or antigen-binding fragments thereof that specifically bind to CD3 and one or more antibodies or antigen-binding fragments thereof that specifically bind to CD28, ICOS, CD27, CD40, CD137, the like, or combinations thereof.

In some embodiments, the particle comprises a receptor molecule that is an MHC-tetramer (MHC class I or class II) and the immune co-stimulatory molecules or the immune response molecules encapsulated within and/or attached to the surface of the particle. In such an embodiment, the primary recognition would be dictated by antigen-specificity by the MHC tetramer, while the stimulation of such targeted cells by the immune co-stimulatory molecules or the immune response molecules would occur later. Consequently, only Ag-specific cells would be co-stimulated, allowing for lower magnitude of Cytokine Release Syndrome.

In some embodiments, the particle comprises between about 1 and about 100,000,000 copies of the one or more biomolecules (e.g., including immune response biomolecules and immune co-stimulatory biomolecules). In some embodiments, the particle is approximately the same size as the target cell and comprises between about 500 and 100,000,000 copies of the one or more biomolecules. In some embodiments, the particle is approximately about 5 μm to about 200 μm and comprises between about 500 and 100,000,000 copies of the one or more biomolecules. In some embodiments, the particle has a diameter of at least 5 μm. In some embodiments, the particle comprises at least the same number of the one or more biomolecules as binding sites of the target cell. In some embodiments, the particle comprises more of the one or more biomolecules than the corresponding binding sites of the target cell. In some embodiments, the particle comprises at least 1, at least 10, at least 100, at least 1,000, at least 10,000, at least 100,000, at least 1,000,000, at least 10,000,000, or at least 100,000,000 copies of the one or more biomolecules.

In some embodiments, the biomolecules are attached to the surface of the particle. In some embodiments, the biomolecules are in the matrix of the particle itself (e.g., encapsulated or embedded within the particle). In some embodiments, the particle is engineered to degrade to provide such biomolecule to the target cell. The rate of degradation can be modulated to provide slow degradation of the particle and thus slow release of the biomolecule to the target cell. In some embodiments, the biomolecules are attached to both the surface of the particle and in the matrix of the particle. In some embodiments, the biomolecules on the surface and in the matrix of the particle are the same. In some embodiments, the biomolecules on the surface and in the matrix of the particle are different. In some embodiments, the biomolecules on the surface and in the matrix of the particle are different and the components of the matrix dissolve at different rates.

### Exemplary Immune Co-Stimulatory and Immune Response Biomolecules

In some embodiments, the particle of the disclosure comprises a CD28 receptor immune response biomolecule. CD28 receptor is a surface glycoprotein that is present in 80% of peripheral T cells in humans and is present in both quiescent and activated T cells. Combined with TCR engagement, CD28 ligation on T cells induces the production of interleukin-2 (IL-2). Secreted IL-2 is an important factor for ex vivo T cell expansion. A canonical form of human CD28 protein is provided, for example, in Uniprot database under Uniprot ID P10747, with the amino acid sequence of SEQ ID NO: 5.

As noted in other portions of this disclosure, in some embodiments, the immune response biomolecule is connected to the synthetic particle via a linker. In some embodiments, the linker is an immune co-stimulatory biomolecule that activates CD28 receptor signaling. In some embodiments, the linker is attached to the extracellular portion of the immune response biomolecule.

In some embodiments, the extracellular portion of the CD28 protein comprises an immunoglobulin variable like region corresponding to amino acids 28-137 of SEQ ID NO: 5.

In some embodiments, the synthetic particles of the present disclosure comprises an immune response biomolecule comprising at least 80%, 85%, 90%, 95%, 98%, 99%, or 100% sequence identity with amino acids 28-220 of SEQ ID NO: 5, including all ranges and subranges therebetween, or the extracellular portion thereof.

In some embodiments, the particle of the disclosure comprises an immune co-stimulatory biomolecule that activates CD28 receptor signaling. In some embodiments, the biomolecule that activates CD28 receptor signaling is a CD28 ligand, a ligand mimic, an antibody, a peptide, an aptamer, or a small molecule. In some embodiments, the immune co-stimulatory biomolecule that activates CD28 receptor signaling binds CD28 receptor with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules. In some embodiments, the biomolecule that activates CD28 receptor signaling comprises an antibody that specifically binds CD28 receptor, or the antigen-binding fragment thereof. In some embodiments, the biomolecule that activates CD28 receptor signaling is B7-1 (CD80) or B7-2 (CD86), or a functional fragment thereof. Non-limiting examples of immune co-stimulatory biomolecules that activate CD28 receptor signaling includes those antibodies, aptamers, ligand proteins disclosed in Pastor et al. Mol Ther Nucleic Acids. (2013) June 11; 2:e98, U.S. Application Publication Nos. 20200268845; 20030232323; 20140271677; 20040137577; 20020106730; 20100303811 and International Application Publication Nos. WO2014089009; WO1995003408, the contents of each of which are hereby incorporated by reference in their entireties for all purposes.

In some embodiments, the biomolecule that activates CD28 receptor signaling comprises an anti-CD28 receptor antibody or antigen binding fragment thereof. In some embodiments, the anti-CD28 receptor antibody or antigen binding fragment thereof binds CD28 (e.g., in a domain outside the basolateral domain) and co-stimulates T cells in a TCR-dependent mechanism. In some embodiments, the anti-CD28 receptor antibody or antigen binding fragment thereof is a “superagonistic” one that binds CD28 through the basolateral domain resulting in a polyclonal activation of T lymphocytes even in the absence of TCR stimulation. In some embodiments, the superagonistic anti-CD28 antibody is TGN1412 (TAB08). Additional non-limiting examples of anti-CD28 antibodies and antigen binding fragments thereof are disclosed in Poirier et al. (2012) American Journal of Transplantation 12(7): 1682-1690, Cell Immunol. 2005 July-August; 236(1-2): 154-60, the contents of each of which are hereby incorporated by reference in their entireties for all purposes. In some embodiments, the anti-CD28 antibody or antigen binding fragment thereof comprises, or is derived form, a mouse IgG1 monoclonal antibody (clone CD28.2) available from BioLegend® (e.g., Catalog #302901 or 302902).

In some embodiments, the biomolecule that activates CD28 receptor signaling comprises a B7-1 (CD80) ligand or a functional fragment thereof. The canonical form of B7-1 (CD80) in Homo sapiens is provided, for example, in Uniprot database under Uniprot ID P33681. In some embodiments, the functional fragment of the B7-1 (CD80) comprises the part of its extracellular domain responsible for binding to and activating the CD28 receptor. In some embodiments, the B7-1 (CD80) or the functional fragment thereof comprises an amino acid sequence at least 80%, at least 85%, at least 90%, at least 95%, at least 96%, at least 97%, at least 98%, at least 99%, or 100% identical to SEQ ID NO: 6, including all ranges and subranges therebetween. In some embodiments, the B7-1 (CD80) or the functional fragment comprises the extracellular portion of the B7-1 (CD80) protein. In some embodiments, the B7-1 (CD80) or the functional fragment thereof comprises an amino acid sequence at least 80%, at least 85%, at least 90%, at least 95%, at least 96%, at least 97%, at least 98%, at least 99%, or 100% identical to amino acids 35-230 of SEQ ID NO: 6, including all ranges and subranges therebetween.

In some embodiments, the biomolecule that activates CD28 receptor signaling comprises a B7-2 (CD86) ligand or a functional fragment thereof. The canonical form of B7-2 (CD86) in Homo sapiens is provided, for example, in Uniprot database under Uniprot ID P42081. In some embodiments, the functional fragment of the B7-2 (CD86) comprises the part of its extracellular domain responsible for binding to and activating the CD28 receptor. In some embodiments, the B7-2 (CD86) or the functional fragment thereof comprises an amino acid sequence at least 80%, at least 85%, at least 90%, at least 95%, at least 96%, at least 97%, at least 98%, at least 99%, or 100% identical to SEQ ID NO: 7, including all ranges and subranges therebetween. In some embodiments, the B7-2 (CD86) or the functional fragment comprises the extracellular portion of the B7-2 (CD86) protein. In some embodiments, the B7-2 (CD86) or the functional fragment thereof comprises an amino acid sequence at least 80%, at least 85%, at least 90%, at least 95%, at least 96%, at least 97%, at least 98%, at least 99%, or 100% identical to amino acids 33-225 of SEQ ID NO: 7, including all ranges and subranges therebetween.

In some embodiments, the particle of the disclosure comprises a 4-1BB receptor immune response biomolecule. 4-1BB receptor, also known as CD137, is a member of the TNF-receptor (TNFR) superfamily and participates in the regulation of immune response. A representative human 4-1BB receptor is provided, for example, in Uniprot database under Uniprot ID Q07011, with the amino acid sequence of SEQ ID NO: 3. As noted in other portions of this disclosure, in some embodiments, the immune response biomolecule is connected to the synthetic particle via a linker. In some embodiments, the linker is an immune co-stimulatory biomolecule that activate 4-1BB receptor signaling. In some embodiments, the linker is attached to the extracellular portion of the immune response biomolecule. In some embodiments, the extracellular portion of the 4-1BB receptor comprises four cysteine-rich domains (CRD) in the region corresponding to amino acids 24-159 of SEQ ID NO: 3. In some embodiments, the synthetic particle of population of particles of the present disclosure comprises an immune response biomolecule comprising at least 80%, 85%, 90%, 95%, 98%, 99%, or 100% sequence identity with amino acids 24-255 of SEQ ID NO: 3, including all ranges and subranges therebetween, or the extracellular portion thereof.

In some embodiments, the particle of the disclosure comprises an immune co-stimulatory biomolecule that activates 4-1BB receptor signaling. In some embodiments, the biomolecule that activates 4-1BB receptor signaling is a 4-1BB ligand, a ligand mimic, an antibody, a peptide, an aptamer, or a small molecule. In some embodiments, the immune co-stimulatory biomolecule that activates 4-1BB receptor signaling binds 4-1BB receptor with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules. In some embodiments, the biomolecule that activates 4-1BB receptor signaling comprises an antibody that specifically binds 4-1BB receptor, or the antigen-binding fragment thereof. In some embodiments, the biomolecule that activates 4-1BB receptor signaling is a 4-1BB ligand (4-1BBL) or a functional fragment thereof.

In some embodiments, the biomolecule that activates 4-1BB receptor signaling comprises an anti-4-1BB receptor antibody or antigen binding fragment thereof. In some embodiments, the biomolecule that activates 4-1BB receptor signaling is selected from the group consisting of PRS-343 (Cinrebafusp alfa), RG7827 (RO7122290), ADG106, INBRX-105/ES101, CTX-471, Gen1046/BNT311, MCLA-145, RG6076 (RO7227166), MP0310, Gen1042/BNT312, AGEN2373, LVGN6051, ATOR-1017, STA551, ND-021/NM21-1480, GNC-038 (Emfizatamab), DSP107, FS120, FS222, HOT-1030, ABL503/TJ-L14B, IBI319, GNC-039, EU101, CB307, ABL111 (TJ-CD4B, TJ-CLDN4B, TJ033721), GNC-035, PRS-344/S095012, BI 765179, QL301/QLF31907, ATG-101/YN-051/Ori-Bs-001, BT7480, PM1003, YH004, LBL-024, PM1032, HLX35/BNA035, HBM7008, ABL105/YH32367, BGB-B167, ADG206, PE0116, a functional fragment thereof, a derivative thereof, a variant thereof, a biosimilar thereof, and any combinations thereof. Non-limiting examples of biomolecules that activate 4-1BB receptor signaling are provided in Claus et al., MAbs. 2023 January-December; 15(1):2167189, the content of which is incorporated by reference in its entirety for all purposes.

In some embodiments, the biomolecule that activates 4-1BB receptor signaling comprises a 4-1BB ligand (4-1BBL) or the functional fragment thereof. The canonical form of 4-1BBL in Homo sapiens is provided, for example, in Uniprot database under Uniprot ID P41273. In some embodiments, the functional fragment of the 4-1BBL comprises the part of its extracellular domain responsible for binding to and activating the 4-1BB receptor. In some embodiments, the 4-1BBL or the functional fragment thereof comprises an amino acid sequence at least 80%, at least 85%, at least 90%, at least 95%, at least 96%, at least 97%, at least 98%, at least 99%, or 100% identical to SEQ ID NO: 1, including all ranges and subranges therebetween. In some embodiments, the 4-1BBL or the functional fragment comprises the extracellular portion of the 4-1BBL protein. Thus, in some embodiments, the 4-1BBL or the functional fragment thereof comprises an amino acid sequence at least 80%, at least 85%, at least 90%, at least 95%, at least 96%, at least 97%, at least 98%, at least 99%, or 100% identical to amino acids 80-244 of SEQ ID NO: 1, including all ranges and subranges therebetween. In some embodiments, the 4-1BBL or the functional fragment thereof comprises an amino acid sequence at least 80%, at least 85%, at least 90%, at least 95%, at least 96%, at least 97%, at least 98%, at least 99%, or 100% identical to amino acids 50-254 of SEQ ID NO: 1, including all ranges and subranges therebetween.

In some embodiments, the particle of the disclosure comprises an OX40 receptor immune response biomolecule. OX40 receptor is also known as CD134 or Tumor necrosis factor receptor superfamily member 4 (TNFRSF4). A representative human OX40 receptor is provided, for example, in Uniprot database under Uniprot ID P43489, with the amino acid sequence of SEQ ID NO: 4. As noted in other portions of this disclosure, in some embodiments, the immune response biomolecule is connected to the synthetic particle via a linker. In some embodiments, the linker is an immune co-stimulatory biomolecule that activates OX40 receptor signaling. In some embodiments, the linker is attached to the extracellular portion of the immune response biomolecule. In some embodiments, the extracellular portion of the OX40 receptor comprises cysteine-rich domains (CRD) in the region corresponding to amino acids 30-167 of SEQ ID NO: 4. In some embodiments, the synthetic particle of population of particles of the present disclosure comprises an immune response biomolecule comprising at least 80%, 85%, 90%, 95%, 98%, 99%, or 100% sequence identity with amino acids 30-277 of SEQ ID NO: 4, including all ranges and subranges therebetween, or the extracellular portion thereof.

In some embodiments, the particle of the disclosure comprises an immune co-stimulatory biomolecule that activates OX40 receptor signaling. In some embodiments, the biomolecule that activates OX40 receptor signaling is an OX40 ligand, a ligand mimic, an antibody, a peptide, an aptamer, or a small molecule. In some embodiments, the immune co-stimulatory biomolecule that activates OX40 receptor signaling binds OX40 receptor with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules. In some embodiments, the biomolecule that activates OX40 receptor signaling comprises an antibody that specifically binds OX40 receptor, or the antigen-binding fragment thereof. In some embodiments, the biomolecule that activates OX40 receptor signaling is an OX40 ligand (OX40L) or a functional fragment thereof.

In some embodiments, the biomolecule that activates OX40 receptor signaling comprises an anti-OX40 receptor antibody or antigen binding fragment thereof. In some embodiments, the biomolecule that activates OX40 receptor signaling is selected from the group consisting of MED10562, MED16469, MED16383 (Efizonerimod), tavolixizumab, GSK3174998, MOXR0916, PF-04518600 (Ivuxolimab), BMS-986178, Creative Biolabs MOM-18455, a functional fragment thereof, a derivative thereof, a variant thereof, a biosimilar thereof, and any combinations thereof. Additional examples of biomolecules that activate OX40 receptor signaling can be found, for example, in Yadav and Redmond, Curr Oncol Rep. 2022 July; 24(7):951-960, the content of which is incorporated by reference in its entirety for all purposes.

In some embodiments, the biomolecule that activates OX40 receptor signaling comprises an OX40 ligand (OX40L) or a functional fragment thereof. The canonical form of OX40L in Homo sapiens is provided, for example, in Uniprot database under Uniprot ID P23510. In some embodiments, the functional fragment of the OX40L comprises the part of its extracellular domain responsible for binding to and activating the OX40 receptor. In some embodiments, the OX40L or the functional fragment thereof comprises an amino acid sequence at least 80%, at least 85%, at least 90%, at least 95%, at least 96%, at least 97%, at least 98%, at least 99%, or 100% identical to SEQ ID NO: 2, including all ranges and subranges therebetween. In some embodiments, the OX40L or the functional fragment comprises the extracellular portion of the OX40L protein. Thus, in some embodiments, the OX40L or the functional fragment thereof comprises an amino acid sequence at least 80%, at least 85%, at least 90%, at least 95%, at least 96%, at least 97%, at least 98%, at least 99%, or 100% identical to amino acids 61-174 of SEQ ID NO: 2, including all ranges and subranges therebetween. In some embodiments, the OX40L or the functional fragment thereof comprises an amino acid sequence at least 80%, at least 85%, at least 90%, at least 95%, at least 96%, at least 97%, at least 98%, at least 99%, or 100% identical to amino acids 51-183 of SEQ ID NO: 2, including all ranges and subranges therebetween.

As noted in other parts of this disclosure, in some embodiments, the particle of the disclosure comprises an immune response biomolecule from Table 9. In some embodiments, the immune response biomolecule is connected to the synthetic particle via a linker. In some embodiments, the linker is the corresponding immune co-stimulatory biomolecule from Table 9. In some embodiments, the linker is attached to the extracellular portion of the immune response biomolecule. In some embodiments, the synthetic particle of population of particles of the present disclosure comprises an immune response biomolecule comprising at least 80%, 85%, 90%, 95%, 98%, 99%, or 100% sequence identity with any of the immune response biomolecules of Table 9, or the extracellular portion thereof.

In some embodiments, the particle of the disclosure comprises an immune co-stimulatory biomolecule, such as those disclosed in Table 9. In some embodiments, the immune co-stimulatory biomolecule is a ligand, a ligand mimic, an antibody, a peptide, an aptamer, or a small molecule binding to any of the immune response biomolecules of Table 9. In some embodiments, the immune co-stimulatory biomolecule binds to the immune response biomolecule with a dissociation constant (Kd) of less than 10 μM, less than 1 μM, less than 100 nM, less than 10 nM, or less than 1 nM, as measured by surface plasmon resonance (SPR) method using a sensor chip that contains immobilized immune co-stimulatory biomolecules. In some embodiments, the immune co-stimulatory biomolecule comprises an antibody or antigen-binding fragments thereof that specifically binds the corresponding immune response biomolecule of table 9. In some embodiments, the immune co-stimulatory biomolecule comprises an amino acid sequence at least 80%, at least 85%, at least 90%, at least 95%, at least 96%, at least 97%, at least 98%, at least 99%, or 100% identical to any of the immune co-stimulatory biomolecule sequences of Table 9, including all ranges and subranges therebetween.

### Optional Components of the Synthetic Particles

In some embodiments, the particle of the disclosure comprises an antigen of the immune cell in addition to the at least one immune co-stimulatory biomolecule and/or immune response biomolecule. Persons having skill in the art will be able to identify, make, and use various antigens for use in the presently disclosed technology. For example, for synthetic particles that aim to activate CAR-T cells, the particles may comprise the antigen that activates the chimeric antigen receptor (CAR) expressed by the engineered T cells. For activating anti-CD19 CAR-T cells, the particle of the disclosure may comprise CD19 antigen in addition to the at least one immune co-stimulatory biomolecule and/or immune response biomolecule.

In some embodiments, the particle comprises one or more molecules that support cell growth and/or stimulate target cell proliferation or activation. These molecules include, but are not limited to, cytokines, growth factors, cytokine receptors, extracellular matrix, transcription factors, secreted polypeptides and other molecules, and growth factor receptors, or fragments thereof. In some embodiments, the particle comprises a fibroblast growth factor (bFGF), an acidic fibroblast growth factor (aFGF), an epidermal growth factor (EGF), insulin-like growth factor 1 (IGF-I), insulin-like growth factor-II (IGF-II), a platelet-derived growth factor-AB (PDGF), a vascular endothelial cell growth factor (VEGF), activin-A, a bone morphogenic protein (BMP), a chemokine, a morphogen, a neutralizing antibody, a heregulin, an interferon, a macrophage-derived cytokine, an interleukin, an interleukin receptor, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, 11-23, IL-24, IL-25, IL-26, IL-28, IL-29, IL-30, IL-31, IL-32, IL-33, IL-34, IL-35, IL-36, IL-37, IL-38, tumor necrosis factor, TNFα, TNFβ, TNFR1, TNFR2, IFAR1, IFAR2, TGFR1, TGFR2, FGF, granulocyte macrophage colony-stimulating factor, chemokines (e.g. CCL1, CCL2, CCL3, CCL, CCL5, and CXCL8), CD27 ligand (CD27L), CD40L, CD137L, TNF-related apoptosis-inducing ligand (TRAIL), TNF-related activation-induced cytokine (TRANCE), TNF-related weak inducer of apoptosis (TWEAK), B cell activating factor (BAFF), LIGHT (homologous to lymphotoxin, exhibits inducible expression and competes with herpes simplex virus glycoprotein D for binding to herpesvirus entry mediator, a receptor expressed on T lymphocytes), TNF-like cytokine 1A (TL1A), glucocorticoid-induced TNF receptor-related protein ligand (GITRL), transforming growth factor α (TGF-α), TGF-β, vascular endothelial growth factor (VEGF), nerve growth factor (NGF), macrophage colony-stimulating factor (M-CSF), granulocyte-macrophage colony-stimulating factor (GM-CSF), IFN-α, IFN-β, and IFN-γ.

In some embodiments, the particle of the present disclosure comprises one or more polypeptides that promote expansion of a particular T cell subtype while simultaneously inhibiting the development of the other subset. In some embodiments, the polypeptide that promotes expansion of a particular T cell subtype is a cytokine. In some embodiments, the cytokine is an interleukin, interferon, lymphotoxin, a member of the TNF superfamily, or an antibody or antigen-binding fragment thereof that binds to one of the foregoing. In some embodiments, the cytokine is selected from a list including, but not limited to, IL-1, IL-2, IL-4, IL-5, IL-7, IL-10, IL-12, IL-15, IL-17, IL-21, interferon γ, IFN alpha, IFN beta, lymphotoxin α, TNFα, TNFβ, and any combination thereof.

In some embodiments, the particle of the present disclosure comprises one or more T cell homeostasis factors. In some embodiments, the T cell homeostasis factor is selected from a list including, but not limited to, transforming growth factor β (TGF-β), or agonists thereof, mimetics thereof, variants thereof, functional fragments thereof, or a combination thereof. In some embodiments, the T cell homeostasis factor is IL-2, an agonist, mimetic, variant, or functional fragment or a combination thereof.

In some embodiments, the particle of the disclosure comprises an interleukin and a cell surface molecule. In some embodiments, the particle comprises at least two interleukins and a cell surface molecule. In some embodiments, the particle comprises IL-2, IL-15, IL-21, CD137L, and CD137 (TNFRSF9; 4-1BB). In some embodiments, the particle comprises IL-15, IL-21, CD137L, and CD137 and activates NK cells.

In some embodiments, the synthetic particles are used to eliminate a pathogenic subset of T-cells, B-cells, NK cells, or other immune cells. For example, to eliminate pathogenic T-cells in auto-immune disease. For example, a synthetic particle specific to a B-cell which makes antibodies against autoantigens as in Systemic Lupus Erythematosus (SLE). This results in elimination of B-cells that produce antibodies against various auto antigens.

In some embodiments, the particle comprises one or more components of the extracellular matrix. In some embodiments, the particle provides physical support for the target cells.

In some embodiments, the particle comprises growth factor, cytokines or hormone precursors that must be processed by a protease to release the active growth factor. In some embodiments, the corresponding proteases capable of producing the active growth factor may be added to the growth media, naturally secreted by the target cells or included in the composition of the particles.

### Population of Synthetic Particles

In some embodiments, the disclosure provides a population of synthetic particles that contain, overall, (i) the 4-1BB receptor and/or a biomolecule that activates 4-1BB receptor signaling; (ii) the OX40 receptor and/or a biomolecule that activates OX40 receptor signaling; and (iii) the CD28 receptor and/or a biomolecule that activates CD28 signaling.

In some embodiments, the disclosure provides a population of synthetic particles that contain, overall, (i) a biomolecule that activates 4-1BB receptor signaling; (ii) a biomolecule that activates OX40 receptor signaling; and (iii) a biomolecule that activates CD28 receptor signaling (together referred to as the “core immune co-stimulatory biomolecules”).

In some embodiments, the population of synthetic particles contain all these three types of immune co-stimulatory biomolecules. For example, in some embodiments, individual particles within the population of synthetic particles may comprise all three core immune co-stimulatory biomolecules. In some embodiments, these three core immune co-stimulatory biomolecules may not be present on the same particles. In some embodiments, all or a part of the particles in the population comprise at most two, or at most one of the core immune co-stimulatory biomolecules, but the population of synthetic particles overall contain all three core immune co-stimulatory biomolecules, which may be achieved by mixing different synthetic particles that contain different types of biomolecules. Thus, a population of synthetic particles comprising all three core immune co-stimulatory biomolecules may comprise three distinct sub-populations of particles, each sub-population comprising only one type of core immune co-stimulatory biomolecule.

In some embodiments, the population of synthetic particles further comprise an antigen of the immune cell (e.g., CD19 for anti-CD19 CAR-T cells). In some embodiments, such antigen of the immune cells is present on the synthetic particles comprising at least one immune co-stimulatory biomolecule.

In some embodiments, the disclosure provides a population of synthetic particles that contain the following immune response biomolecules: (i) a 4-1BB; (ii) an OX40 receptor; and (iii) a CD28 receptor (together referred to as the “core immune response biomolecules”). In some embodiments, the immune response biomolecules are tethered to an immune cell. In some embodiments, the immune response biomolecules are attached to the synthetic particles via linkers. In some embodiments, the immune response biomolecules are non-covalently attached to the linkers. In some embodiments, the linkers are immune co-stimulator biomolecules.

In some embodiments, the population of synthetic particles contain all these three types of core immune response biomolecules. For example, in some embodiments, individual particles within the population of synthetic particles may comprise all three core immune response biomolecules attached via corresponding linkers, and wherein the immune response biomolecules are tethered to an immune cell. In some embodiments, these three immune response biomolecules may not be present on the same particles. In some embodiments, all or a part of the particles in the population comprise at most two, or at most one of the core immune response biomolecules, but the population of synthetic particles overall contain all three core immune response biomolecules, which may be achieved by mixing different synthetic particles that contain different types of linkers with the immune cells that these immune response biomolecules are tethered to. Thus, a population of synthetic particles comprising all three core immune response biomolecules may comprise three distinct sub-populations of particles, each sub-population comprising only one type of immune response biomolecule.

In some embodiments, an immune cell tethered with all three core immune response biomolecules are attached to a synthetic particle via the corresponding linkers. In some embodiments, an immune cell tethered with all three core immune response biomolecules are attached to multiple synthetic particles, with each synthetic particle attaching to one or two types of the core immune response biomolecules.

In some embodiments, the population of synthetic particles further comprise an antigen of the immune cell (e.g., CD19 for anti-CD19 CAR-T cells). In some embodiments, such antigen interacts with the corresponding immune receptor present on the immune cells.

### Target Cell

In some embodiments, particles of the disclosure support the proliferation, activation, and/or survival of target cells.

A target cell can be virtually any type of cell, including prokaryotic and eukaryotic cells. In some embodiments, the target cell is as described above or in one of Tables 2 and 6-7.

In some embodiments, a target cell is an immune cell. Non-limiting examples of immune cells include B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor infiltrating (TIL) cells, gene modified immune cells including hybridomas, drug modified immune cells, and derivatives, precursors, or progenitors of any of the cell types listed herein.

Though the present disclosure is described with reference to immune cells, and in particular, to a T cell, the disclosure is not intended to be so limited in its scope of application. The present disclosure may be used for plasma cells, lymphocytes, immune cells, biomolecule presenting cells (e.g., dendritic cells, macrophages, B cells), naive B cells, memory B cells, naïve T cells, memory T cells, chimeric antigen receptor T cells (CAR-T cells), regulatory T cells, cytotoxic T cells, NK cells, or any other appropriate cell. Additionally, the method may be used for any number of cells or analytes, such as one, at least one, a plurality, etc.

In some embodiments, a target cell encompasses all cells of a particular class of cell with shared properties. For example, a target cell can be a lymphocyte, including NK cells, T cells, and B cells. A target cell can be an activated lymphocyte.

In some embodiments, the T cell stimulated and/or expanded and or depleted/removed by the particle of the present disclosure is selected from the nonlimiting group consisting of natural killer (NK) cells, CD3+ T cells, CD4+ T cells, CD8+ T cells, and regulatory T cells (Treg), or a combination thereof. In some embodiments, the T cell is a helper T cell. In some embodiments, the T cell is a cytotoxic T cell. In some embodiments, the T cell is a Th1 or a Th2 cell. In some embodiments, the T cell is a recombinant T cell. In some embodiments, the recombinant T cell is a CAR-T cell. In embodiments, T cells depleted/removed by the particles of the present disclosure are CD25+ regulatory T cells and/or CD4+ T cells.

In some embodiments, the T cell is freshly collected from a subject. In some embodiments, the T cell is a cultured cell line. In some embodiments, the T cell is an established cell line. In some embodiments, the T cell is cultured from a preserved or frozen sample.

In some embodiments, the particles of the present disclosure induce the expansion, proliferation, and/or activation of any appropriate immune cell (e.g., T cell). In some embodiments, the immune cell (e.g., T cell) does not expand, proliferate, and/or activate in culture without the synthetic particles. In some embodiments, the immune cell (e.g., T cell) does not expand, proliferate, and/or activate well in culture without the synthetic particles.

In some embodiments, the immune cells (e.g., T cells), or subsets thereof are eliminated as a consequence of incubating with the synthetic particles.

In some embodiments, the immune cells (e.g., T cells) are derived from any appropriate source within an animal. The animals from which the T cells are harvested may be vertebrate or invertebrate, mammalian or non-mammalian, human or non-human. Examples of animal sources include, but are not limited to, primates, rodents, canines, felines, equines, bovines, and porcines.

In some embodiments, a target cell is a primary cell, cultured cell, established cell, normal cell, transformed cell, infected cell, stably transfected cell, transiently transfected cell, proliferating cell, or terminally differentiated cells.

In some embodiments, a target cell is a primary neuronal cell. A variety of neurons can be target cells. As non-limiting examples, a target cell can be a primary neuron; established neuron; transformed neuron; stably transfected neuron; or motor or sensory neuron.

In other embodiments, a target cell is selected from the group consisting of primary lymphocytes, monocytes, and granulocytes.

Suitable prokaryotic target cells include, but are not limited to, bacteria such as E. coli, various Bacillus species, and the extremophile bacteria such as thermophiles.

Suitable eukaryotic target cells include, but are not limited to, fungi such as yeast and filamentous fungi, including species of Saccharomyces, Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tilapia, tuna, carp, flounder, halibut, swordfish, cod and zebrafish. Suitable bird cells include, but are not limited to, those of chickens, ducks, quail, pheasants and turkeys, and other jungle fowl or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes.

Suitable target cells also include those cell types implicated in a wide variety of disease conditions, even while in a non-diseased state. Accordingly, suitable eukaryotic cell types include, but are not limited to, tumor cells of all types (e.g., melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as hematopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. In certain embodiments, the cells are primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In some embodiments, a target cell is a tumor microvesicle or tumor macrovesicle. Tumor microvesicles, also known as tumor-secreted microvesicles or tumor-secreted exosomes, can be found in circulating blood and may have immune-suppressive activities. Tumor microvesicles typically range in size from 30-200 nm in diameter. Larger tumor micro vesicles may be referred to as tumor macro vesicles and can range in size from 3-10 μm in diameter.

In some embodiments, the target cell is a stem cell. In some embodiments, the stem cell is, without limitation, an embryonic stem cell, an ICM/epiblast cell, a primitive ectoderm cell, a primordial germ cell, a cancer cell, or a teratocarcinoma cell.

In some embodiments, the stem cell is a pluripotent stem cell, a totipotent stem cell, a multipotent stem cell, an oligopotent, or a unipotent stem cell. In some embodiments, the pluripotent stem cell is an embryonic stem cell. In some embodiments, the stem cell is an undifferentiated pluripotent stem cell. In some embodiments, the totipotent stem cell is, without limitation, an embryonic stem cell, a neural stem cell, a bone marrow stem cell, a hematopoietic stem cell, a cardiomyocyte, a neuron, an astrocyte, a muscle cell, or a connective tissue cell. In some embodiments, the multipotent stem cell is, without limitation, a myeloid progenitor cell, or a lymphoid progenitor cell. In some embodiments, the stem cell is an induced pluripotent stem cell (iSPC). In some embodiments, the stem cell is an adult stem cell. In some embodiments, the stem cell is an undifferentiated pluripotent stem cell. In some embodiments, the stem cell is a mammalian stem cell. In some embodiments, the stem cell is a primate stem cell. In some embodiments, the stem cell is a human stem cell.

In some embodiments, the stem cells are derived from any source within an animal. For example, stem cells may be harvested from embryos, or any primordial germ layer therein, from placental or chorion tissue, or from more mature tissue such as adult stem cells including, but not limited to adipose, bone marrow, nervous tissue, mammary tissue, liver tissue, pancreas, epithelial, respiratory, gonadal and muscle tissue. In some embodiments, the stem cells are placental- or chorionic-derived stem cells.

In some embodiments, the present disclosure contemplates using differentiable cells from any animal capable of generating differentiable cells, e.g., pancreatic type cells such as beta cells. The animals from which the differentiable cells are harvested may be vertebrate or invertebrate, mammalian or non-mammalian, human or non-human. Examples of animal sources include, but are not limited to, primates, rodents, canines, felines, equines, bovines, and porcines.

In some embodiments, the target cell is a blood cell. In some embodiments, the target cell is a peripheral blood mononuclear cell (PMBC). In some embodiments, the peripheral blood mononuclear cell is a lymphocyte, a monocyte, or a dendritic cell. In some embodiments, the lymphocyte is a T-cell, B-cell, or NK cell. In some embodiments, the target cell is a natural killer (NK) cell.

In certain embodiments of the present disclosure, the cell culture is enriched. The term “enriched” refers to a cell culture that contains at least 10%, 15%, 20%, 25%, 30%, 35%, 40%, 45%, 50%, 55%, 60%, 65%, 70%, 75%, 80%, 85%, 90%, 95%, or 100% of the desired cell lineage.

As used herein, the term “substantially undifferentiated” cell culture refers to a population of stem cells comprising at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein [EGFP]) to one or more markers indicative of a desired undifferentiated state can be used to determine how many cells of a given stem cell population are undifferentiated. For purposes of making this assessment, one or more cell surface markers correlated with an undifferentiated state (e.g., SSEA-4, Tra-1-60, and Tra-1-81), as well as the typical pluripotent stem cell transcription factor marker, Oct-4, can be detected. Telomerase reverse transcriptase (TERT) activity and alkaline phosphatase can also be assayed. In the context of primate stem cells, positive and/or negative selection can be used to detect, for example, by immuno-staining or employing a reporter gene (e.g., EGFP), the expression (or lack thereof) of certain markers (e.g., Oct-4, SSEA-4, Tra-1-60, Tra-1-81, SSEA-1, SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells. Also, undifferentiated cells described herein have typical stem cell morphology which is well described in the art.

Cell types including but not limited to various cell lines such as CHO, HEK-293, BHK-21, NS0, MDCK, VERO, MRC-S, W1-38 and Sp2/0 Mouse Myeloma (hybridomas). Table 6 and Table 7 each provides other cell types for use with the particles described herein.

TABLE 6

| | |
|---|---|
| keratinocyte of epidermis | Pancreatic acinar cell |
| basal cell of epidermis | Paneth cell of small intestine |
| keratinocyte of fingernails and toenails | pneumocyte of lung |
| basal cell of nail bed | Clara cell of lung |
| hair shaft cells | anterior pituitary cells |
| medullary hair shaft cells | Somatotropes |
| cortical hair shaft cells | Lactotropes |
| cuticular hair shaft cells | Thyrotropes |
| hair-root sheath cells | Gonadotropes |
| cuticular hair-root sheath cells | Corticotropes |
| hair-root sheath cells of Huxley's layer | melanocyte-stimulating hormone |
| hair-root sheath cells of Henle's layer | Magnocellular neurosecretory cells secreting: |
| external hair-root sheath cells | Gut and respiratory tract cells secreting: |
| hair matrix cell (stem cell) | Thyroid gland cells |
| surface epithelial cell of stratified squamous | thyroid epithelial cell |
| epithelium of tongue | |
| surface epithelial cell of stratified squamous | parafollicular cell |
| epithelium of oral cavity | |
| surface epithelial cell of stratified squamous | Parathyroid gland cells |
| epithelium of esophagus | |
| surface epithelial cell of stratified squamous | Parathyroid chief cell |
| epithelium of anal canal | |
| surface epithelial cell of stratified squamous | Oxyphil cell |
| epithelium of distal urethra | |
| surface epithelial cell of stratified squamous | Adrenal gland cells |
| epithelium of vagina | |
| basal cell of these epithelia | chromaffin cells |
| cell of urinary epithelium | secreting steroid hormones (mineral |
| | corticoids and gluco corticoids) |
| cells of salivary gland | Leydig cell of testes secreting testosterone |
| Mucous cells of salivary gland | Theca interna cell of ovarian |
| | follicle secreting estrogen |
| Serous cell of salivary gland | Corpus luteum cell of ruptured ovarian follicle |
| | secreting progesterone |
| cell of von Ebner's gland in tongue | Granulosa lutein cells |
| cell of mammary gland | Theca lutein cells |
| cell of lacrimal gland | Juxtaglomerular cell (renin secretion) |
| cell of ceruminous gland of ear | Macula densa cell of kidney |
| cell of eccrine sweat gland | Peripolar cell of kidney |
| cell of eccrine sweat gland | Mesangial cell of kidney |
| cell of apocrine sweat gland | epidermal keratinocyte |
| cell of gland of Moll in eyelid | Epidermal basal cell |
| cell of sebaceous gland | Keratinocyte of fingernails and toenails |
| cell of Bowman's gland in nose | Nail bed basal cell (stem cell) |
| cell of Brunner's gland in duodenum | Medullary hair shaft cell |
| cell of seminal vesicle | Cortical hair shaft cell |
| cell of prostate gland | Cuticular hair shaft cell |
| cell of bulbourethral gland | Cuticular hair root sheath cell |
| cell of Bartholin's gland | Hair root sheath cell of Huxley's layer |
| cell of gland of Littre | Hair root sheath cell of Henle's layer |
| cell of endometrium of uterus | External hair root sheath cell |
| isolated goblet cell of respiratory and digestive | Hair matrix cell (stem cell) |
| tracts | |
| mucous cell of lining of stomach | epithelial cell of stratified squamous |
| | epithelium of cornea, |
| zymogenic cell of gastric gland | epithelial cell of stratified squamous |
| | epithelium of tongue |
| oxyntic cell of gastric gland | epithelial cell of stratified squamous |
| | epithelium of oral cavity |
| acinar cell of pancreas | epithelial cell of stratified squamous |
| | epithelium of esophagus |
| Paneth cell of small intestine | epithelial cell of stratified squamous |
| | epithelium of anal canal |
| type II pneumocyte of lung | epithelial cell of stratified squamous |
| | epithelium of distalurethra |
| Clara cell of lung | epithelial cell of stratified squamous |
| | epithelium of vagina |
| cells of anterior pituitary | basal cell (stem cell) of epithelia of cornea |
| cell of intermediate pituitary | basal cell (stem cell) of epithelia of tongue |
| cells of posterior pituitary | basal cell (stem cell) of epithelia of oral cavity |
| cells of gut and respiratory tract | basal cell (stem cell) of epithelia of esophagus |
| cells of thyroid gland | basal cell (stem cell) of epithelia of anal canal |
| cells of parathyroid gland | basal cell (stem cell) of epithelia of distal urethra |
| cells of adrenal gland | basal cell (stem cell) of epithelia of vagina |
| steroid hormones | Urinary epithelium cell |
| cells of gonads | Auditory inner hair cell of organ of Corti |
| cells of juxtaglomerular apparatus of kidney | Auditory outer hair cell of organ of Corti |
| juxtaglomerular cell | basal cell of olfactory epithelium |
| macula | Cold-sensitive primary sensory neurons |
| densa cell | Heat-sensitive primary sensory neurons |
| peripolar cell | Merkel cell of epidermis (touch sensor) |
| mesangial cell | Olfactory receptor neuron |
| brush border cell of intestine | Pain-sensitive primary sensory neurons (various |
| | types) |
| striated duct cell of exocrine glands | Photoreceptor cells of retina in eye: |
| gall bladder epithelial cell | Photoreceptor rod cells |
| brush border cell of proximal tubule of kidney | Photoreceptor blue-sensitive cone cell of eye |
| distal tubule cell of kidney | Photoreceptor green-sensitive cone cell of eye |
| Non ciliated cell of ductulus efferens | Photoreceptor red-sensitive cone cell of eye |
| epididymal principal cell | Proprioceptive primary sensory neurons |
| epididymal basal cell | Touch-sensitive primary sensory neurons |
| hepatocyte | Type I carotid body cell |
| white fat cell | Type II carotid body cell |
| brown fat cell | Type I hair cell of vestibular system of ear |
| lipocyte of liver | Type II hair cell of vestibular system of ear |
| type I pneumocyte | Type I taste bud cell |
| pancreatic duct cell | Cholinergic neural cell |
| parietal cell of kidney glomerulus | Adrenergic neural cell |
| podocyte of kidney glomerulus | Peptidergic neural cell |
| cell of thin segment of loop of Henle | Inner pillar cell of organ of Corti |
| collecting duct cell (in kidney) | Outer pillar cell of organ of Corti |
| duct cell of seminal vesicle | Inner phalangeal cell of organ of Corti |
| duct cell of prostate gland | Outer phalangeal cell of organ of Corti |
| vascular endothelial cells of blood vessels and | Border cell of organ of Corti |
| lymphatics | |
| fenestrated vascular endothelial cells | Hensen cell of organ of Corti |
| continuous vascular endothelial cells | Vestibular apparatus supporting cell |
| splenic vascular endothelial cells | Taste bud supporting cell |
| synovial cell | Olfactory epithelium supporting cell |
| serosal cell | Schwann cell |
| squamous cell lining perilymphatic space of ear | Satellite glial cell |
| cells lining endolymphatic space of ear | Enteric glial cell |
| squamous cell | Astrocyte |
| columnar cells of endolymphatic sac | Neuron cells |
| “dark” cell | Oligodendrocyte |
| vestibular membrane cell | Spindle neuron |
| stria vascularis basal cell | Anterior lens epithelial cell |
| stria vascularis marginal cell | Crystallin-containing lens fiber cell |
| cell of Claudius | Hepatocyte |
| cell of Boettcher | Adipocytes (white fat cell, brown fat cell, liver |
| | lipocyte) |
| choroid plexus cell | Kidney parietal cell |
| squamous cell of pia-arachnoid | Kidney glomerulus podocyte |
| cells of ciliary epithelium of eye | Kidney proximal tubule brush border cell |
| corneal “endothelial” cell | Loop of Henle thin segment cell |
| Ciliated Cells of respiratory tract | Kidney distal tubule cell |
| Ciliated Cells of oviduct and of endometrium of | Kidney collecting duct cell |
| uterus | |
| Ciliated Cells of rete testis and ductulus efferens | Type I pneumocyte |
| Ciliated Cells of central nervous system | Pancreatic duct cell |
| epithelial | Nonstriated duct cell |
| ameloblast | principal cell |
| nonepithelial | Intercalated cell |
| chondrocytes | Duct cell |
| osteoblast/osteocyte | Intestinal brush border cell |
| osteoprogenitor cell | Exocrine gland striated duct cell |
| hyalocyte of vitreous body of eye | Gall bladder epithelial cell |
| stellate cell of perilymphatic space of ear | Ductulus efferens non ciliated cell |
| skeletal muscle cells | Epididymal principal cell |
| heart muscle cells | Epididymal basal cell |
| smooth muscle cells (various) | Ameloblast epithelial cell |
| myoepithelial cells | Planum semilunatum epithelial cell of vestibular |
| | system of ear |
| red blood cell | Organ of Corti interdental epithelial cell |
| megakaryocyte | Loose connective tissue fibroblasts |
| macrophages and related cells | Corneal fibroblasts (corneal keratocytes) |
| neutrophil | Tendon fibroblasts |
| eosinophil | Bone marrow reticular tissue fibroblasts |
| basophil | nonepithelial fibroblasts |
| mast cell | Pericyte |
| T lymphocyte | Nucleus pulposus cell of intervertebral disc |
| B lymphocyte | Cementoblast/cementocyte |
| photoreceptors (rods, cones, and can be blue | Odontoblast/odontocyte |
| sensitive, green sensitive, red sensitive) | |
| inner hair cell of organ of Corti | Hyaline cartilage chondrocyte |
| outer hair cell of organ of Corti | Fibrocartilage chondrocyte |
| type I hair cell of vestibular apparatus of ear | Elastic cartilage chondrocyte |
| type II hair cell of vestibular apparatus of ear | Osteoblast/osteocyte |
| type II taste bud cell | Osteoprogenitor cell |
| olfactory neuron | Hyalocyte of vitreous body of eye |
| basal cell of olfactory epithelium | Stellate cell of perilymphatic space of ear |
| carotid body cell type I | Hepatic stellate cell (Ito cell) |
| carotid body cell type II | Pancreatic stellate cell |
| Merkel cell of epidermis | skeletal muscle Cell |
| primary sensory neurons specialized for touch | Red skeletal muscle cell (slow) |
| (various) | |
| primary sensory neurons specialized for temperature - | White skeletal muscle cell (fast) |
| cold sensitive | |
| primary sensory neurons specialized for temperature - | Intermediate skeletal muscle cell |
| heat sensitive | |
| primary sensory neurons specialized for pain | nuclear bag cell of muscle spindle |
| (various) | |
| proprioceptive primary sensory neurons (various) | nuclear chain cell of muscle spindle |
| Autonomic Neurons | Satellite cell (stem cell) |
| inner pillar cell | Heart muscle cells |
| outer pillar cell | Ordinary heart muscle cell |
| inner phalangeal cell | Nodal heart muscle cell |
| outer phalangeal cell | Purkinje fiber cell |
| border cell | Smooth muscle cell |
| Hensen cell | Myoepithelial cell of iris |
| supporting cell of vestibular apparatus | Myoepithelial cell of exocrine glands |
| supporting cell of taste bud (type I taste bud cell) | Erythrocyte |
| supporting cell of olfactory epithelium | Megakaryocyte |
| Schwann cell | Monocyte |
| satellite cell (encapsulating peripheral nerve cell | Connective tissue macrophage |
| bodies) | |
| enteric glial cell | Epidermal Langerhans cell |
| neurons | Osteoclast (in bone) |
| glial cells | Dendritic cell (in lymphoid tissues) |
| anterior lens epithelial cell | Microglial cell (in central nervous system) |
| lens fiber (crystallin-containing cell) | Neutrophil granulocyte |
| melanocyte | Eosinophil granulocyte |
| retinal pigmented epithelial cell | Basophil granulocyte |
| oogonium/oocyte | Hybridoma cell |
| spermatocyte | Mast cell |
| spermatogonium (stem cell for spermatocyte) | Helper T cell |
| ovarian follicle cell | Suppressor T cell |
| Sertoli cell (in testis) | Cytotoxic T cell |
| thymus epithelial cell | Natural Killer T cell |
| Salivary gland mucous cell | B cell |
| Salivary gland number 1 | Natural killer cell |
| Von Ebner's gland cell in tongue | Reticulocyte |
| Mammary gland cell | Stem cells and committed progenitors for |
| | the blood and immune system (various types) |
| Lacrimal gland cell | Oogonium/Oocyte |
| Ceruminous gland cell in ear | Spermatid |
| Eccrine sweat gland dark cell | Spermatocyte |
| Eccrine sweat gland clear cell | Spermatogonium cell |
| Apocrine sweat gland cell | Spermatozoon |
| Gland of Moll cell in eyelid | Ovarian follicle cell |
| Sebaceous gland cell | Thymus epithelial cell |
| Bowman's gland cell in nose | Interstitial kidney cells |
| Brunner's gland cell in duodenum | |
| Seminal vesicle cell | |
| Prostate gland cell | |
| Bulbourethral gland cell | |
| Bartholin's gland cell | |
| Gland of Littre cell | |
| Uterus endometrium cell | |
| goblet cell of respiratory and digestive tracts | |
| Stomach lining mucous cell | |
| Gastric gland zymogenic cell | |
| Gastric gland oxyntic cell | |

TABLE 7
Keratinizing Epithelial Cells
keratinocyte of epidermis (=differentiating epidermal cell)
basal cell of epidermis (stem cell)
keratinocyte of fingernails and toenails
basal cell of nail bed (stem cell)
hair shaft cells
medullary
cortical
cuticular
hair-root sheath cells
Cuticular root sheath cells
root sheath cells of Huxley's layer
root sheath cells of Henle's layer
external root sheath cells
hair matrix cell (stem cell)
Cells of Wet Stratified Barrier Epithelia
surface epithelial cell of stratified squamous epithelium of cornea, tongue, oral
cavity, esophagus, anal canal, distal urethra, vagina
basal cell of these epithelia (stem cell)
cell of urinary epithelium (lining bladder and urinary ducts)
Epithelial Cells Specialized for Exocrine Secretion
cells of salivary gland
mucous cell (secretion rich in polysaccharide)
serous cell (secretion rich in glycoprotein enzymes)
cell of von Ebner's gland in tongue (secretion to wash over taste buds)
cell of mammary gland, secreting milk
cell of lacrimal gland, secreting tears
cell of ceruminous gland of ear, secreting wax
cell of eccrine sweat gland, secreting glycoproteins (dark cell)
cell of eccrine sweat gland, secreting small molecules (clear cell)
cell of apocrine sweat gland (odoriferous secretion, sex-hormone sensitive)
cell of gland of Moll in eyelid (specialized sweat gland)
cell of sebaceous gland, secreting lipid-rich sebum
cell of Bowman's gland in nose (secretion to wash over olfactory epithelium)
cell of Brunner's gland in duodenum, secreting alkaline solution of mucus and enzymes
cell of seminal vesicle, secreting components of seminal fluid, including fructose (as fuel for swimming sperm)
cell of prostate gland, secreting other components of seminal fluid
cell of bulbourethral gland, secreting mucus
cell of Bartholin's gland, secreting vaginal lubricant
cell of gland of Littre, secreting mucus
cell of endometrium of uterus, secreting mainly carbohydrates
isolated goblet cell of respiratory and digestive tracts, secreting mucus
mucous cell of lining of stomach
zymogenic cell of gastric gland, secreting pepsinogen
oxyntic cell of gastric gland, secreting HCl
acinar cell of pancreas, secreting digestive enzymes and bicarbonate
Paneth cell of small intestine, secreting lysozyme
type II pneumocyte of lung, secreting surfactant
Clara cell of lung (function unknown)
Cells Specialized for Secretion of Hormones
cells of anterior pituitary, secreting growth hormone, follicle-stimulating hormone, luteinizing hormone, prolactin,
adrenocorticotropic hormone, and/or thyroid-stimulating hormone
cell of intermediate pituitary, secreting melanocyte-stimulating hormone
cells of posterior pituitary, secreting oxytocin and/or vasopressin
cells of gut and respiratory tract, secreting serotonin, endorphin, somatostatin, gastrin, secretin, cholecystokinin,
insulin, glucagon, and/or bombesin
cells of thyroid gland, secreting
thyroid hormone
calcitonin
cells of parathyroid gland, secreting
parathyroid hormone
oxyphil cell (function unknown)
cells of adrenal gland, secreting
epinephrine
norepinephrine
steroid hormones
mineralocorticoids
glucocorticoids
cells of gonads, secreting
testosterone (Leydig cell of testis)
estrogen (theca interna cell of ovarian follicle)
progesterone (corpus luteum cell of ruptured ovarian follicle)
cells of juxtaglomerular apparatus of kidney
juxtaglomerular cell (secreting renin)
macula densa cell
peripolar cell
mesangial cell
Epithelial Absorptive Cells in Gut, Exocrine Glands, and Urogenital Tract
brush border cell of intestine (with microvilli)
striated duct cell of exocrine glands
gall bladder epithelial cell
brush border cell of proximal tubule of kidney
distal tubule cell of kidney
Non ciliated cell of ductulus efferens
epididymal principal cell
epididymal basal cell
Cells Specialized for Metabolism and Storage
hepatocyte (liver cell)
fat cells
white fat
brown fat
lipocyte of liver
Epithelial Cells Serving Primarily a Barrier Function, Lining the
Lung, Gut, Exocrine Glands, and Urogenital Tract
type I pneumocyte (lining air space of lung)
pancreatic duct cell (centroacinar cell)
nonstriated duct cell of sweat gland, salivary gland, mammary gland, etc.
(various)
parietal cell of kidney glomerulus
podocyte of kidney glomerulus
cell of thin segment of loop of Henle (in kidney)
collecting duct cell (in kidney)
duct cell of seminal vesicle, prostate gland, etc. (various)
Epithelial Cells Lining Closed Internal Body Cavities
vascular endothelial cells of blood vessels and lymphatics
fenestrated
continuous
splenic
synovial cell (lining joint cavities, secreting largely hyaluronic acid)
serosal cell (lining peritoneal, pleural, and pericardial cavities)
squamous cell lining perilymphatic space of ear
cells lining endolymphatic space of ear
squamous cell
columnar cells of endolymphatic sac
with microvilli
without microvilli
“dark” cell
vestibular membrane cell
stria vascularis basal cell
stria vascularis marginal cell
cell of Claudius
cell of Boettcher
choroid plexus cell (secreting cerebrospinal fluid)
squamous cell of pia-arachnoid
cells of ciliary epithelium of eye
pigmented
nonpigmented
corneal “endothelial” cell
Ciliated Cells with Propulsive Function
Ciliated Cells of respiratory tract
Ciliated Cells of oviduct and of endometrium of uterus (in female)
Ciliated Cells of rete testis and ductulus efferens (in male)
Ciliated Cells of central nervous system (ependymal cell lining brain cavities)
Cells Specialized for Secretion of Extracellular Matrix
epithelial
ameloblast (secreting enamel of tooth)
planum semilunatum cell of vestibular apparatus of ear
(secreting proteoglycan)
interdental cell of organ of Corti (secreting tectorial “membrane” covering
hair cells of organ of Corti)
nonepithelial (connective tissue)
fibroblasts (various-of loose connective tissue, of cornea, of
tendon, of reticular tissue of bone marrow, etc.)
pericyte of blood capillary
nucleus pulposus cell of intervertebral disc
cementoblast/cementocyte (secreting bonelike cementum of
root of tooth)
odontoblast/odontocyte (secreting dentin of tooth)
chondrocytes
of hyaline cartilage
of fibrocartilage
of elastic cartilage
osteoblast/osteocyte
osteoprogenitor cell (stem cell of osteoblasts)
hyalocyte of vitreous body of eye
stellate cell of perilymphatic space of ear
Contractile Cells
skeletal muscle cells
red (slow)
white (fast)
intermediate
muscle spindle-nuclear bag
muscle spindle-nuclear chain
satellite cell (stem cell)
heart muscle cells
ordinary
nodal
Purkinje fiber
smooth muscle cells (various)
myoepithelial cells
of iris
of exocrine glands
Cells of Blood and Immune System
red blood cell
megakaryocyte
macrophages and related cells
monocyte
connective-tissue macrophage (various)
Langerhans cell (in epidermis)
osteoclast (in bone)
dendritic cell (in lymphoid tissues)
microglial cell (in central nervous system)
neutrophil
eosinophil
basophil
mast cell
T lymphocyte
helper T cell
suppressor T cell
killer T cell
B lymphocyte
IgM
IgG
IgA
IgE
killer cell
stem cells and committed progenitors for the blood and
immune system (various)
Sensory Transducers
photoreceptors
rod
cones
blue sensitive
green sensitive
red sensitive
hearing
inner hair cell of organ of Corti
outer hair cell of organ of Corti
acceleration and gravity
type I hair cell of vestibular apparatus of ear
type II hair cell of vestibular apparatus of ear
taste
type II taste bud cell
smell
olfactory neuron
basal cell of olfactory epithelium (stem cell for olfactory neurons)
blood pH
carotid body cell
type I
type II
touch
Merkel cell of epidermis
primary sensory neurons specialized for touch (various)
temperature
primary sensory neurons specialized for temperature
cold sensitive
heat sensitive
pain
primary sensory neurons specialized for pain (various)
configurations and forces in musculoskeletal system
proprioceptive primary sensory neurons (various)
Autonomic Neurons
cholinergic (various)
adrenergic (various)
peptidergic (various)
Supporting Cells of Sense Organs and of Peripheral Neurons
supporting cells of organ of Corti
inner pillar cell
outer pillar cell
inner phalangeal cell
outer phalangeal cell
border cell
Hensen cell
supporting cell of vestibular apparatus
supporting cell of taste bud (type I taste bud cell)
supporting cell of olfactory epithelium
Schwann cell
satellite cell (encapsulating peripheral nerve cell bodies)
enteric glial cell
Neurons and Glial Cells of Central Nervous System
neurons (huge variety of types-still poorly classified)
glial cells
astrocyte (various)
oligodendrocyte
Lens Cells
anterior lens epithelial cell
lens fiber (crystallin-containing cell)
Pigment Cells
melanocyte
retinal pigmented epithelial cell
Germ Cells
oogonium/oocyte
spermatocyte
spermatogonium (stem cell for spermatocyte)
Nurse Cells
ovarian follicle cell
Sertoli cell (in testis)
thymus epithelial cell
Exocrine secretory epithelial cells
Salivary gland mucous cell (polysaccharide-rich secretion)
Salivary gland number 1 (glycoprotein enzyme-rich secretion)
Von Ebner's gland cell in tongue (washes taste buds)
Mammary gland cell (milk secretion)
Lacrimal gland cell (tear secretion)
Ceruminous gland cell in ear (earwax secretion)
Eccrine sweat gland dark cell (glycoprotein secretion)
Eccrine sweat gland clear cell (small molecule secretion)
Apocrine sweat gland cell (odoriferous secretion, sex-hormone sensitive)
Gland of Moll cell in eyelid (specialized sweat gland)
Sebaceous gland cell (lipid-rich sebum secretion)
Bowman's gland cell in nose (washes olfactory epithelium)
Brunner's gland cell in duodenum (enzymes and alkaline mucus)
Seminal vesicle cell (secretes seminal fluid components, including fructose for swimming sperm)
Prostate gland cell (secretes seminal fluid components)
Bulbourethral gland cell (mucus secretion)
Bartholin's gland cell (vaginal lubricant secretion)
Gland of Littre cell (mucus secretion)
Uterus endometrium cell (carbohydrate secretion)
Isolated goblet cell of respiratory and digestive tracts (mucus secretion)
Stomach lining mucous cell (mucus secretion)
Gastric gland zymogenic cell (pepsinogen secretion)
Gastric gland oxyntic cell (hydrochloric acid secretion)
Pancreatic acinar cell (bicarbonate and digestive enzyme secretion)
Paneth cell of small intestine (lysozyme secretion)
Type II pneumocyte of lung (surfactant secretion)
Clara cell of lung
Hormone secreting cells
Anterior pituitary cells
Somatotropes
Lactotropes
Thyrotropes
Gonadotropes
Corticotropes
Intermediate pituitary cell, secreting melanocyte-stimulating hormone
Magnocellular neurosecretory cells
secreting oxytocin
secreting vasopressin
Gut and respiratory tract cells
secreting serotonin
secreting endorphin
secreting somatostatin
secreting gastrin
secreting secretin
secreting cholecystokinin
secreting insulin
secreting glucagon
secreting bombesin
Thyroid gland cells
thyroid epithelial cell
parafollicular cell
Parathyroid gland cells
Parathyroid chief cell
Oxyphil cell
Adrenal gland cells
chromaffin cells
secreting steroid hormones (mineral corticoids and gluco corticoids)
Leydig cell of testes secreting testosterone
Theca interna cell of ovarian follicle secreting estrogen
Corpus luteum cell of ruptured ovarian follicle secreting progesterone
Granulosa lutein cells
Theca lutein cells
Juxtaglomerular cell (renin secretion)
Macula densa cell of kidney
Peripolar cell of kidney
Mesangial cell of kidney
Derived primarily from ectoderm
Integumentary system
Keratinizing epithelial cells
Epidermal keratinocyte (differentiating epidermal cell)
Epidermal basal cell (stem cell)
Keratinocyte of fingernails and toenails
Nail bed basal cell (stem cell)
Medullary hair shaft cell
Cortical hair shaft cell
Cuticular hair shaft cell
Cuticular hair root sheath cell
Hair root sheath cell of Huxley's layer
Hair root sheath cell of Henle's layer
External hair root sheath cell
Hair matrix cell (stem cell)
Wet stratified barrier epithelial cells
Surface epithelial cell of stratified squamous epithelium of cornea, tongue, oral cavity, esophagus, anal
canal, distalurethra and vagina
basal cell (stem cell) of epithelia of cornea, tongue, oral cavity, esophagus, anal canal, distal urethra and
vagina
Urinary epithelium cell (lining urinary bladder and urinary ducts)
Nervous system
There are nerve cells, also known as neurons, present in our human body. They are branched out. These cells
make up nervous tissue. A neuron consists of a cell body with a nucleus and cytoplasm, from which long thin hair-
like parts arise.
Sensory transducer cells
Auditory inner hair cell of organ of Corti
Auditory outer hair cell of organ of Corti
Basal cell of olfactory epithelium (stem cell for olfactory neurons)
Cold-sensitive primary sensory neurons
Heat-sensitive primary sensory neurons
Merkel cell of epidermis (touch sensor)
Olfactory receptor neuron
Pain-sensitive primary sensory neurons (various types)
Photoreceptor cells of retina in eye:
Photoreceptor rod cells
Photoreceptor blue-sensitive cone cell of eye
Photoreceptor green-sensitive cone cell of eye
Photoreceptor red-sensitive cone cell of eye
Proprioceptive primary sensory neurons (various types)
Touch-sensitive primary sensory neurons (various types)
Type I carotid body cell (blood pH sensor)
Type II carotid body cell (blood pH sensor)
Type I hair cell of vestibular system of ear (acceleration and gravity)
Type II hair cell of vestibular system of ear (acceleration and gravity)
Type I taste bud cell
Autonomic neuron cells
Cholinergic neural cell
Adrenergic neural cell
Peptidergic neural cell
Sense organ and peripheral neuron supporting cells
Inner pillar cell of organ of Corti
Outer pillar cell of organ of Corti
Inner phalangeal cell of organ of Corti
Outer phalangeal cell of organ of Corti
Border cell of organ of Corti
Hensen cell of organ of Corti
Vestibular apparatus supporting cell
Taste bud supporting cell
Olfactory epithelium supporting cell
Schwann cell
Satellite glial cell (encapsulating peripheral nerve cell bodies)
Enteric glial cell
Central nervous system neurons and glial cells
Astrocyte (various types)
Neuron cells (large variety of types, still poorly classified)
Oligodendrocyte
Spindle neuron
Lens cells
Anterior lens epithelial cell
Crystallin-containing lens fiber cell
Derived primarily from mesoderm
Metabolism and storage cells
Hepatocyte (liver cell)
Adipocytes:
White fat cell
Brown fat cell
Liver lipocyte
Barrier function cells (lung, gut, exocrine
glands and urogenital tract)
Kidney
Kidney parietal cell
Kidney glomerulus podocyte
Kidney proximal tubule brush border cell
Loop of Henle thin segment cell
Kidney distal tubule cell
Kidney collecting duct cell[disambiguation needed]
Type I pneumocyte (lining air space of lung cell)
Pancreatic duct cell (centroacinar cell)
Nonstriated duct cell (of sweat gland, salivary gland, mammary gland, etc.)
principal cell
Intercalated cell
Duct cell (of seminal vesicle, prostate gland, etc.)
Intestinal brush border cell (with microvilli)
Exocrine gland striated duct cell
Gall bladder epithelial cell
Ductulus efferens non ciliated cell
Epididymal principal cell
Epididymal basal cell
Extracellular matrix cells
Ameloblast epithelial cell (tooth enamel secretion)
Planum semilunatum epithelial cell of vestibular system of ear (proteoglycan secretion)
Organ of Corti interdental epithelial cell (secreting tectorial membrane covering hair cells)
Loose connective tissue fibroblasts
Corneal fibroblasts (corneal keratocytes)
Tendon fibroblasts
Bone marrow reticular tissue fibroblasts
Other nonepithelial fibroblasts
Pericyte
Nucleus pulposus cell of intervertebral disc
Cementoblast/cementocyte (tooth root bonelike ewan cell secretion)
Odontoblast/odontocyte (tooth dentin secretion)
Hyaline cartilage chondrocyte
Fibrocartilage chondrocyte
Elastic cartilage chondrocyte
Osteoblast/osteocyte
Osteoprogenitor cell (stem cell of osteoblasts)
Hyalocyte of vitreous body of eye
Stellate cell of perilymphatic space of ear
Hepatic stellate cell (Ito cell)
Pancreatic stelle cell
Contractile cells
skeletal muscle Cell
Red skeletal muscle cell (slow)
White skeletal muscle cell (fast)
Intermediate skeletal muscle cell
nuclear bag cell of muscle spindle
nuclear chain cell of muscle spindle
Satellite cell (stem cell)
Heart muscle cells
Ordinary heart muscle cell
Nodal heart muscle cell
Purkinje fiber cell
Smooth muscle cell (various types)
Myoepithelial cell of iris
Myoepithelial cell of exocrine glands
Blood and immune system cells
Erythrocyte (red blood cell)
Megakaryocyte (platelet precursor)
Monocyte (white blood cell )
Connective tissue macrophage (various types)
Epidermal Langerhans cell
Osteoclast (in bone)
Dendritic cell (in lymphoid tissues)
Microglial cell (in central nervous system)
Neutrophil granulocyte
Eosinophil granulocyte
Basophil granulocyte
Hybridoma cell
Mast cell
Helper T cell
Suppressor T cell
Cytotoxic T cell
Natural Killer T cell
B cell
Natural killer cell
Reticulocyte
Stem cells and committed progenitors for the blood and immune system (various types)
Germ cells
Oogonium/Oocyte
Spermatid
Spermatocyte
Spermatogonium cell (stem cell for spermatocyte)
Spermatozoon
Nurse cells
Ovarian follicle cell
Sertoli cell (in testis)
Thymus epithelial cell
Interstitial cells
Interstitial kidney cells

Culturing the Particles with Target Cells

In one aspect, particles of the disclosure support cell growth and/or stimulate the proliferation or activation of target cells (e.g., immune cells).

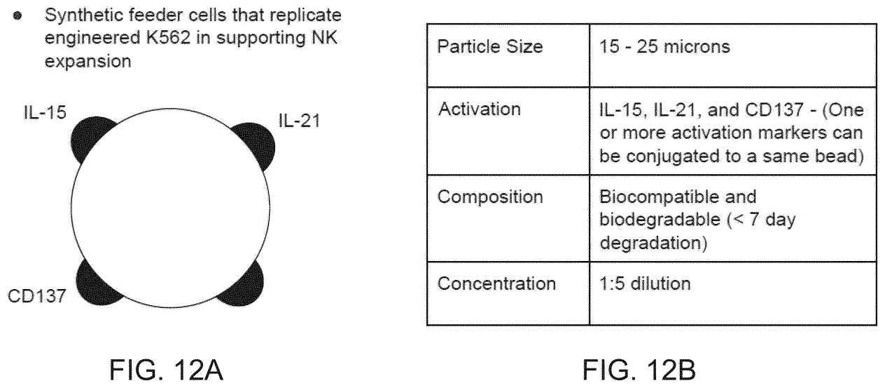

In some embodiments, the synthetic particles of the present disclosure can mimic—act as a synthetic substitute for-feeder cells. Feeder cells support the growth of target cells by releasing biomolecules such as growth factors, adhesion molecules, and/or extracellular matrix to the culture media, but can introduce issues such as viruses and unwanted antigens into the cell culture. Here, as shown in FIG. 10, the present disclosure provides particles that act as feeder cells and provides one or more biomolecules of the disclosure. Such biomolecules may comprise immune co-stimulatory biomolecules, immune response biomolecules, growth factors, adhesion molecules, and/or extracellular matrix. In some embodiments, the particles comprise a polymer matrix and one or more polypeptides or fragments thereof that support the growth of target cells. In some embodiments, the particles comprise one or more polypeptides or fragments (e.g., proliferation analyte) thereof that stimulate the proliferation and/or activation of the target cell. In some embodiments, the target cell does not proliferate in culture without the particles. In some embodiments, the target cell does not proliferate well in culture without the particles.

In one aspect, the present disclosure provides methods of culturing/contacting a target cell (e.g., immune cell) with one or more particles as described herein. In some embodiments, the culturing media is useful in culturing the target cells. In some embodiments, the media is substantially isotonic as compared to the cells being cultured. In some embodiments where undifferentiated stein cells are cultured, the particular medium comprises a base medium and an amount of various factors necessary to support substantially undifferentiated growth of embryonic stem cells. In some embodiments, the base medium comprises salts, essential amino acids, a carbon source that can be metabolized by the target cells, and human serum. In some embodiments, for instance when the target cell is a T cell, the base medium comprises cytokines such as IL-2, TL-7, and IL-15. All these ingredients are supplied in an amount that will support respective target cells.

In some embodiments, the disclosure provides a cell culture composition comprising a target cell, a particle (or a population of particles) as described herein, and wherein the composition is essentially free of feeder cells. In some embodiments, the particle (or the population of particles) comprises one or more of the core immune co-stimulatory biomolecules or core immune response biomolecules. In some embodiments, the population of particles comprise all the core immune co-stimulatory biomolecules (e.g., (i) a biomolecule that activates 4-1BB receptor signaling; (ii) a biomolecule that activates OX40 receptor signaling; and (iii) a biomolecule that activates CD28 receptor signaling). In some embodiments, the population of particles comprise all the immune response biomolecules (e.g., (i) a 4-1BB receptor; (ii) an OX40 receptor; and (iii) a CD28 receptor). In some embodiments, the particle (or the population of particles) further comprises an antigen for the target immune cell.

In some embodiments, the disclosure provides a cell culture composition comprising a target cell, a defined culture media comprising human serum (hS), and a particle (or a population of particles) as described herein, and wherein the composition is essentially free of feeder cells. In some embodiments, the disclosure provides a cell culture composition comprising a natural killer cell, a defined culture media comprising human serum (S), and a particle (or a population of particles) as described herein, and wherein the composition is essentially free of feeder cells. In some embodiments, the disclosure provides a cell culture composition comprising a natural killer cell, a defined culture media comprising human serum (hS), and a particle (or a population of particles) as described herein comprising one or more of an interleukin and/or a member of the tumor necrosis factor superfamily, and wherein the composition is essentially free of feeder cells. In some embodiments, the disclosure provides a cell culture composition comprising a natural killer cell, a defined culture media comprising human serum (hS), and a particle (or a population of particles) as described herein comprising one or more of IL-15, IL-21, CD137L, and/or CD137 and wherein the composition is essentially free of feeder cells. In some embodiments, the disclosure provides a cell culture composition comprising a natural killer cell, a defined culture media comprising human serum (hS), and different particles as described herein comprising one or more of IL-15, IL-21, CD137L, and/or CD137 and wherein the composition is essentially free of feeder cells, in some embodiments, the disclosure provides a particle comprising IL-15, IL-21, CD137L and CD137.

In some embodiments, the disclosure provides a cell culture composition comprising a T cell, a defined culture media comprising human serum (hS), and a particle (or a population of particles) as described herein, and wherein the composition is essentially free of feeder cells. In some embodiments, the disclosure provides a cell culture composition comprising a B cell, a defined culture media comprising human serum (hS), and a CD19-expressing particle (or a population of particles) as described herein, and wherein the composition is essentially free of feeder cells. In some embodiments, the disclosure provides a cell culture composition comprising a T cell, a defined culture media comprising human serum (hS), and a particle (or a population of particles) as described herein comprising one or more antibodies or antigen-binding fragments thereof that specifically bind CD3 and one or more antibodies or antigen-binding fragments thereof that specifically bind CD28, and wherein the composition is essentially free of feeder cells.

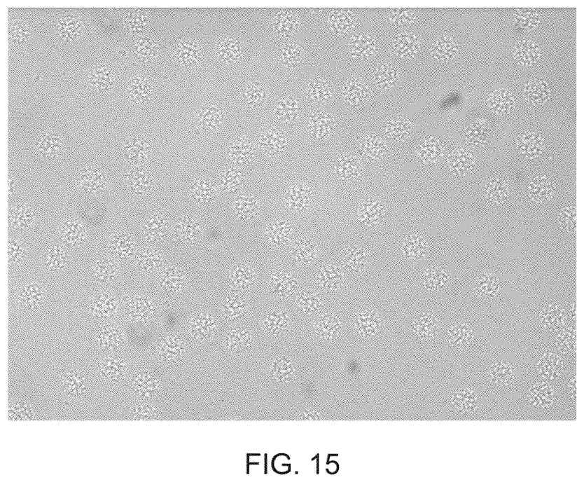

In some embodiments, the disclosure provides a cell culture composition comprising a T cell, a defined culture media comprising human serum (hS), and a particle (or a population of particles), as shown in FIG. 13A and FIG. 13B, comprising one or more antibodies or antigen-binding fragments thereof that specifically bind CD3 and one or more antibodies or antigen-binding fragments thereof that specifically bind CD28, and wherein the composition is essentially free of feeder cells.

In some embodiments, the disclosure provides a cell culture composition comprising a particle, as described herein, and at least one immune cell. In embodiments, the cell culture composition may comprise a particle comprising a matrix comprising a polymerized monomer, said matrix comprising a plurality of micropores and a plurality of macropores and one or more immune co-stimulatory biomolecules or immune response biomolecules, and at least one immune cell. The at least one immune cell may be a target cell selected from one of Tables 2 and 6-7. In some embodiments, the particle interacts with the immune cell through one or more of the immune response biomolecules that binds to the one or more immune co-stimulatory biomolecules on the particle.

In some embodiments, the cells and the particles are cultured in media comprising synthetic media supplements and are serum-free.

In some embodiments, the particles form a single monolayer in the cell culture. In some embodiments, the particles form a multi-layer support in the cell culture.

In some embodiments, the cell culture comprises a single type of particles. In some embodiments, the cell culture comprises a combination of different types of particles.

In some embodiments, the cell culture comprises at least about 1×10¹ particles per mL of cell culture, e.g., at least about 1×10¹, at least about 1×10², at least about 1×10³, at least about 1×10⁴, at least about 1×10⁵, at least about 1×10⁶, at least about 1×10⁷, at least about 1×10⁸, at least about 1×10⁹, at least about 1×10¹⁰, at least about 1×10¹¹, at least about 1×10¹², at least about 1×10¹³, at least about 1×10¹⁴, at least about 1×10¹⁵, at least about 1×10¹⁶, at least about 1×10¹⁷, at least about 1×10¹⁸, at least about 1×10¹⁹, at least about 1×10²⁰, or more. In some embodiments, the cell culture comprises from about 1×10⁵ to about 1×10⁸ particles per mL of cell culture (e.g., 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, including all values and subranges therein). In some embodiments, the cell culture comprises between about 1×10⁵ and about 1×10⁸ particles per mL of cell culture. In some embodiments, the cell culture comprises about 1×10⁵, about 1×10⁶, about 1×10⁷, or about 1×10⁸ particles per mL of cell culture. In some embodiments, the cell culture comprises a similar concentration of particles as feeder cells used in traditional cell culturing methods. In some embodiments, the cell culture comprises a similar concentration of particles as APC cells used in traditional cell culturing methods.

In some embodiments, the particles of the present disclosure are applied to the cell culture at a ratio of about 1:1 to about 1:1000 cells:particles. In some embodiments, the particles are applied to the cell culture at a ratio of about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, about 1:100, about 1:200, about 1:300, about 1:400, about 1:500, about 1:600, about 1:700, about 1:800, about 1:900, or about 1:1000 cells:particles.

In some embodiments, culturing the target cell with a particle of the present disclosure increases target cell proliferation by about 1% to about 10000% compared to culturing of the target cell without the particle. In some embodiments, target cell proliferation is increased by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900%, about 1000%, about 2000%, about 3000%, about 4000%, about 5000%, about 6000%, about 7000%, about 8000%, about 9000%, or about 10000%, including all ranges and subranges therebetween, compared to culturing of the target cell without the particle. In some embodiments, cell proliferation can be at least 100,000× the initial cell population.

In some embodiments, culturing the target cell with a particle of the present disclosure increases target cell activation by about 1% to about 10000% compared to culturing of the target cell without the particle. In some embodiments, target cell proliferation is increased by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900%, about 1000%, about 2000%, about 3000%, about 4000%, about 5000%, about 6000%, about 7000%, about 8000%, about 9000%, or about 10000%, including all ranges and subranges therebetween, compared to culturing of the target cell without the particle. In some embodiments, cell activation can be at least 100,000× the initial cell population.

In some embodiments, the feeder cells can support culturing or proliferation based on proximity of a particle to a cell of interest. In one example, the particle can be conjugated to the cell of interest, whether via direct or indirect conjugation. In another example, the particle can be proximal to but not immediately adjacent to the cell of interest. The particle and the cell of interest can be separated by less than 1 nm, less than 1 micron, less than 1 millimeter, or any appropriate separation distance by which the activation event can still occur.

Culturing or proliferation may be distant from an area in which the cell of interest is located (i.e., culturing or proliferation can occur remotely). The distance can be at least 1 millimeter, at least 1 centimeter, at least 1 meter, etc. For example, the particle may be introduced intramuscularly or intravenously, and the action is in a lymph node or distant immune organ or another target organ. Alternatively, the particle may be introduced on one side of a membrane and the action maybe on another side of a membrane (e.g., via a semi-permeable membrane).

In some embodiments, target cells are cultured with the particles for at least about 30 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, 2 days, 36 hours, 3 days, 4 days, 5 days, 6 days, 7 days, 8 days, 9 days, 10 days, 11 days, 13 days, 14 days, or more, including all values and subranges therein.

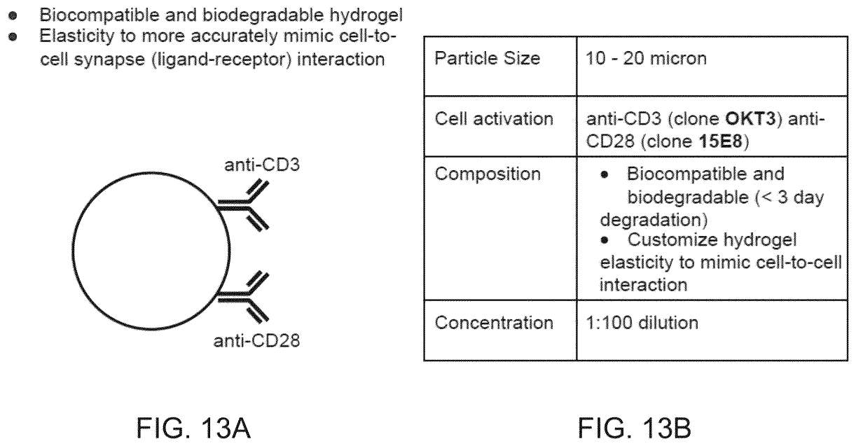

In some embodiments, as shown in FIG. 11, the present disclosure provides particles (comprising a polymer matrix) that act as APCs and comprise one or more immunostimulatory biomolecules (e.g., core immune co-stimulatory biomolecule or core immune response biomolecule) that stimulate the expansion and/or activation of a T cell. In some embodiments, these synthetic biomolecule presenting particles comprise one or more of an activation biomolecule, an immune response biomolecule, an immune co-stimulatory biomolecule, and/or a T cell homeostasis factor.

Furthermore, the present disclosure teaches methods of detecting, inducing, or detecting and inducing activation events including, but not limited to, cell expansion, cell proliferation, cell differentiation, activation maintenance, cell maturation, cell receptor clustering, synapse formation (e.g., between a lymphocyte and a tumor cell), cytokine production, gene expression, protein expression, or any other appropriate occurrence by which the target cell is activated upon recognition of or stimulation by the proper antigen, ligand, antibody, immunoglobulin (e.g., CD3, CD19, CD20, CD28, CD80, CD86, CD69, CD154, CD137, IgM, IgG, IgE, IgA, IgD, or antibodies targeting said biomolecules), toll-like receptors (TLR, such as, for example, TLR1-13), or the like.

In some embodiments, these activation events can be induced based on proximity of a particle to a cell of interest. In some embodiments, the particle can be contacting the cell of interest, whether via direct or indirect conjugation. For example, in some embodiments, the synthetic particles of the present disclosure contact an immune cell via non-covalently linking with an immune response biomolecule still tethered to an immune cell. In some embodiments, the particle can be proximal to but not immediately adjacent to the cell of interest. The particle and the cell of interest can be separated by less than 1 nm, less than 1 micron, less than 1 millimeter, or any appropriate separation distance by which the activation event can still occur.

Action may be distant from an area of introduction of the particle, in which a signal event or cascade event occurs remotely. The distance can be at least 1 millimeter, at least 1 centimeter, at least 1 meter, etc. For example, the particle may be introduced intramuscularly or intravenously, and the action is in a lymph node or distant immune organ or other target organ. Alternatively, the particle may be introduced on one side of a membrane and the action maybe on another side of a membrane (e.g., via a semi-permeable membrane).

In some embodiments, when the synthetic particles of the present disclosure are incubated with immune cells (e.g., T-cells), cells are activated and show early signs of IL-2 secretion and TCR engagement with early-stage and late-stage cell activation markers, CD69 and CD25, respectively, as measured by flow cytometry within 24 hours or 96 hours of culture. In some embodiments, long-term activation is also observed as late as 96 hours after co-culture, indicating a sustained response.

### Adoptive Cell Therapy

Provided are synthetic particles, and cells produced therefrom, for adoptive cell therapy, e.g., adoptive immunotherapy. The cells include immune cells such as those described above, including T cells and NK cells, and in some embodiments, the cells express genetically engineered antigen receptors such as engineered TCRs and/or chimeric antigen receptors (CARs).

The particles are engineered by introducing one or more biomolecules that stimulate T cell expansion and/or activation. The biomolecules may interact with antigen receptors, including engineered T cell receptors (TCRs) and functional non-TCR antigen receptors, such as chimeric antigen receptors (CARs), including activating, stimulatory, and costimulatory CARs, and combinations thereof. In some embodiments, the cells cultured with the synthetic particles disclosed herein express an engineered receptor targeting (e.g., specifically binding to or recognizing) a biomolecule, such as a disease-specific target antigen corresponding to the disease or condition to be treated.

In some embodiments, the adoptive cell therapy is tumor-infiltrating lymphocyte therapy. In tumor infiltrating lymphocyte therapy, naturally occurring T cells that have already infiltrated patients' tumors are harvested and cultured with the synthetic particles described herein to activate and expand them. Activated T cells are then re-infused into patients, where they can then seek out and destroy tumors.

In some embodiments, the adoptive cell therapy is engineered TCR therapy. In TCR therapy, T cells from patients are harvested. The T cells are equipped (engineered) with an appropriate T cell receptor (e.g., as described herein) that enables them to target specific cancer biomolecules. The engineered T cells are then cultured with the synthetic particles described herein to activate and expand them. Activated T cells are then re-infused into patients, where they can then seek out and destroy tumors.

In some embodiments, the adoptive cell therapy is CAR-T cell therapy. In CAR-T cell therapy, T cells from patients are harvested. T cells are collected via apheresis, a procedure during which blood is withdrawn from the body and one or more blood components (such as plasma, platelets or white blood cells) are removed. The remaining blood is then returned to the body. T cells are then reengineered in a laboratory. To this end, the T cells are sent to a laboratory or a drug manufacturing facility where they are genetically engineered, by introducing nucleic acids, RNA, and/or DNA into them, to produce CARs on the surface of the cells. After this reengineering, the T cells are known as CAR-T cells. CARs are proteins that allow the T cells to recognize an antigen on targeted tumor cells. The reengineered CAR-T cells are then cultured with the synthetic particles described herein to activate and expand them. The number of the patient's genetically modified T cells is “expanded” by growing cells in the laboratory. When there are enough of them, these CAR-T cells are frozen and sent to the hospital or center where the patient is being treated. At the hospital or treatment center, the CAR-T cells are thawed and then infused into the patient, where they can then seek out and destroy tumors. CARs can bind to cancer cells even if their antigens are not presented on the surface via major histocompatibility complex, which can render more cancer cells vulnerable to their attacks. Many patients are given a brief course of one or more chemotherapy agents, called “lymphodepletion,” before they receive the infusion of CAR-T cells. CAR-T cells that have been returned to the patient's bloodstream multiply in number. These are the “attacker” cells that will recognize, and attack, cells that have the targeted antigen on their surface.

In some embodiments, the adoptive cell therapy is natural killer (NK) cell therapy.

i. Cells, Cell Preparation, and Culture

In some embodiments, the cells used in this type of therapy are eukaryotic cells, such as mammalian cells, e.g., human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some embodiments, the cells are human cells. The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some embodiments, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naive T (TN) cells, effector T cells (T EFF), memory T cells and sub-types thereof, such as stem cell memory T (T scM), central memory T (TcM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as THI cells, TH2 cells, TH3 cells, THI 7 cells, TH9 cells, TH22 cells, follicular helper T cells, alpha/beta T cells, and delta/gamma T cells.

In some embodiments, one or more of the T cell populations is enriched for or depleted of cells that are positive for (marker+) or express high levels (marker^{high}) of one or more particular markers, such as surface markers, or that are negative for (marker−) or express relatively low levels (marker^{low}) of one or more markers. In some cases, such markers are those that are absent or expressed at relatively low levels on certain populations of T cells (such as nonmemory cells) but are present or expressed at relatively higher levels on certain other populations of T cells (such as memory cells). In some embodiments, the cells (such as the CD8+ cells or the T cells, e.g., CD3+ cells) are enriched for (i.e., positively selected for) cells that are positive or expressing high surface levels of CD45RO, CCR7, CD2S, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L.

In some embodiments, a CD4+ T cell population and a CD8+ T cell sub-population, e.g., a sub-population enriched for central memory (T cM) cells. In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

ii. Cell Preparation

The cells typically are isolated from a sample, such as a biological sample, e.g., one obtained from or derived from a subject. In some embodiments, the subject from which the cell is isolated as one having a particular disease or condition or in need of a cell therapy or to which cell therapy will be administered. The subject in some embodiments is a mammal, such as a human, such as a subject in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some embodiments, the sample from which the cells are derived or isolated is blood or a blood-derived sample or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

iii. Incubation and Culture

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one of the synthetic particles of the present disclosure. In some embodiments, the stimulating conditions or agents include synthetic particles comprising a co-stimulatory or immune response biomolecule capable of enhancing immune activation and/or proliferation. In some embodiments, the synthetic particles of the present disclosure can be used in place of the feeder cells (e.g., non-dividing peripheral blood mononuclear cells) that have been routinely used for ex vivo expansion of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBY-transformed lymphoblastoid cells (LCL) as feeder cells. LCL can be irradiated with gamma rays in the range of about 6000 to 10,000 rads. The LCL feeder cells in some embodiments is provided in any suitable amount, such as a ratio of LCL feeder cells to initial T lymphocytes of at least about 10:1.

In embodiments, antigen-specific T cells, such as antigen specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some embodiments, the methods include assessing expression of one or more markers on the surface of the engineered cells or cells being engineered. In some embodiments, the methods include assessing surface expression of one or more target antigen (e.g., antigen recognized by the genetically engineered antigen receptor) sought to be targeted by the adoptive cell therapy, for example, by affinity-based detection methods such as by flow cytometry. In some embodiments, where the method reveals surface expression of the antigen or other marker, the gene encoding the antigen or other marker is disrupted or expression otherwise repressed for example, using the methods described herein.

Synthetic Particles with Matrix

In some embodiments, the present disclosure teaches synthetic particles with a matrix body. Various synthetic particles of the present disclosure are described herein. In embodiments, the particles of the present disclosure comprise hydrogel particles. A hydrogel is a material comprising a macromolecular three-dimensional network that allows it to swell when in the presence of water, to shrink in the absence of (or by reduction of the amount of) water, but not dissolve in water. The swelling, i.e., the absorption of water, is a consequence of the presence of hydrophilic functional groups attached to or dispersed within the macromolecular network. Crosslinks between adjacent macromolecules result in the aqueous insolubility of these hydrogels. The cross-links may be due to chemical (i.e., covalent) or physical (i.e., Van Der Waal forces, hydrogen-bonding, ionic forces, etc.) bonds. Synthetically prepared hydrogels can be prepared by polymerizing a monomeric material to form a backbone and cross-linking the backbone with a crosslinking agent. As referred to herein, the term “hydrogel” refers to the macromolecular material whether dehydrated or in a hydrated state. A characteristic of a hydrogel that is of particular value is that the material retains the general shape, whether dehydrated or hydrated. Thus, if the hydrogel has an approximately spherical shape in the dehydrated condition, it will be spherical in the hydrated condition. In some embodiments, the particles may be bioreactors, achieved by allowing the porous particles to absorb water, maintain an optimal ion nutrient gradient, and maintain an optimal osmotic pressure which favors cellular growth and cell activation. It is well established in tissue engineering that cell migration is influenced by hydrogel stiffness and rough surface area. Without wishing to be bound by any one theory, the inventors believe that hydrogel particles of the present disclosure lead to the formation of much stronger cell-ligand bonds, thereby leading to enhanced growth and proliferation.

In some embodiments, a hydrogel particle disclosed herein comprises greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, or greater than about 95% water by weight. In some embodiments, a hydrogel particle has a water content of about 10 percent by weight to about 95 percent by weight, or about 20 percent by weight to about 95 percent by weight, or about 30 percent by weight to about 95 percent by weight, or about 40 percent by weight to about 95 percent by weight, or about 50 percent by weight to about 95 percent by weight, or about 60 percent by weight to about 95 percent by weight, or about 70 percent by weight to about 95 percent by weight, or about 80 percent by weight to about 95 percent by weight.

### Degradable Particles

In some embodiments, an individual particle or a plurality thereof comprises a biodegradable polymer. In some embodiments, the biodegradable polymer is a poly(esters) based on polylactide (PLA), polyglycolide (PGA), polycaprolactone (PCL), poly(lactic-co-glycolic) acid (PLGA), and their copolymers. In some embodiments, the biodegradable polymer is a carbohydrate or a protein, or a combination thereof. For example, in some embodiments, a monosaccharide, disaccharide or polysaccharide, (e.g., glucose, sucrose, or maltodextrin) peptide, protein (or domain thereof) is used as a monomer for the particles. Other biodegradable polymers include poly(hydroxyalkanoate)s of the PHB-PHV class, additional poly(ester)s, and natural polymers, for example, modified poly(saccharide)s, e.g., starch, cellulose, and chitosan. In some embodiments, the biocompatible polymer is an adhesion protein, cellulose, a carbohydrate, a starch (e.g., maltodextrin, 2-hydroxyethyl starch, alginic acid), a dextran, a lignin, a polyaminoacid, an amino acid, or chitin. Such biodegradable polymers are available commercially, for example, from Sigma Aldrich (St. Louis, MO).

In some embodiments, the protein comprises only natural amino acids. However, the disclosure is not limited thereto. For example, self-assembling artificial proteins and proteins with non-natural amino acids (e.g., those incorporated into non-ribosomal peptides or synthetically introduced via synthetic approaches, see for example, Zhang et al. (2013). Current Opinion in Structural Biology 23, pp. 581-587, the disclosure of which is incorporated by reference in its entirety for all purposes), or protein domains thereof, can also be used as monomers. The range of non-natural (unnatural) amino acids that can be incorporated into such compositions is well known to those skilled in the art (Zhang et al. (2013). Current Opinion in Structural Biology 23, pp. 581-587; incorporated by reference in its entirety for all purposes). In some embodiments, the biodegradable polymer is used as a co-monomer, i.e., in a mixture of monomers. In some embodiments, the biodegradable polymer is a bifunctional monomer.

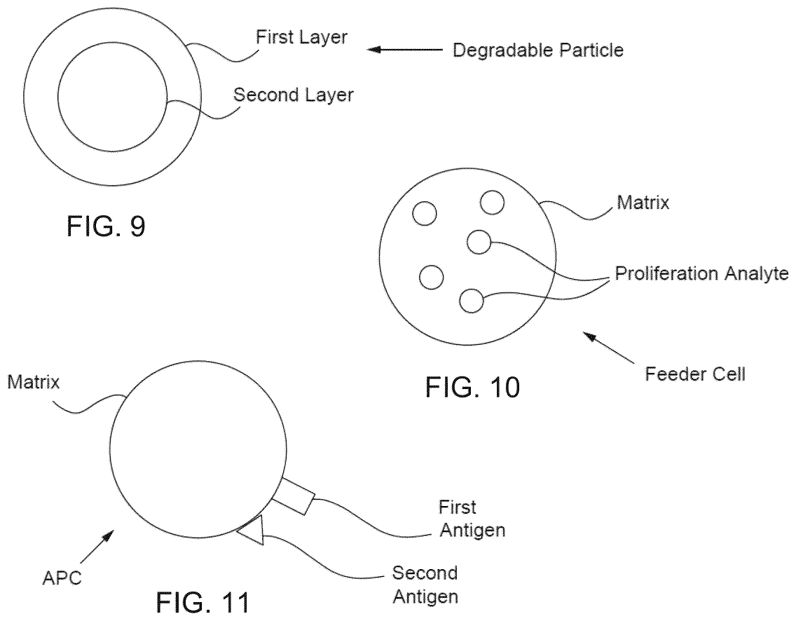

In some embodiments, the particles are engineered to degrade to provide biomolecules to a cell in culture. Degradation can include, without limitation, dissolution (i.e., dissolving) or lysis. The particle can be engineered to have multiple layers, as shown in FIG. 9, with different rates of degradation for at least two of the layers. The particle, whether in its entirety or various layers thereof, can be degraded chemically (e.g., reagents, detergents, bursting, or the like), mechanically (e.g., vibration, acoustic, freeze-thaw, bursting, or the like), or both chemically and mechanically.

The rate of degradation of the entire particles, individual layers of the particles, or groups or subpopulations of a particle population can be fast (i.e., less than 24 hours) or slow (i.e., 24 hours or more). For example, a first layer of a particle can degrade in less than 24 hours and a second layer of the same particle can degrade in 48 hours. As yet another example, a first subpopulation of particles can degrade in less than 1 hour, a second subpopulation of particles can degrade in 24 hours, and a third subpopulation of particles can degrade in one week. The first, second, and third subpopulations form a population of particles.

In some embodiments, a population of particles can include groups or subpopulations of particles having different rates of degradation.

In some embodiments, the particle can be engineered to have pore sizes which correlate to various rates of degradation. The pore sizes can range from 0.1 nm to 1 μm. For example, a first particle can have a first pore size, such that the first particle has a first rate of degradation; and, a second can have a second pore size, such that the particle has a second rate of degradation with the first and second rates of degradation not being equal (e.g., first rate is faster than the second rate; or the first rate is slower than the second rate).

In some embodiments, the particle can be engineered to have a rate of degradation based on a plurality of factors, including, without limitation, pore size, chemical composition (i.e., chemical bonds, monomers, co-monomer), layer composition, the like, and combinations thereof.

In some embodiments, the particle contains disulfide crosslinks enabling the particle to dissolve upon the addition of a reducing agent. In some embodiments, the particle can be dissolved by the addition of a protease. In some embodiments, the growth factors are crosslinked to each other or to the matrix via disulfide crosslinks that may be broken by the addition of a reducing agent, releasing active growth factors. Appropriate reducing agents may include but are not limited to dithiothreitol, Tris(2-carboxyethyl)phosphine hydrochloride, and 2-mercaptoethanol. In some embodiments, the particle comprises only one type of molecule that supports cell growth and/or stimulates target cell proliferation or activation. In some embodiments, the particle comprises only one class of molecule that supports target cell growth and/or stimulates target cell proliferation or activation. In some embodiments, the particle comprises multiple types and/or classes of molecules that support cell growth and/or stimulate target cell proliferation or activation.

### Porous Particles and Porogens

In some embodiments, the present disclosure teaches synthetic particles with one or more pores (granules). In embodiments, the particles of the present disclosure may be particles with enhanced porosity. Compared to non-porous particles, the alteration of pore size distribution allows more surface area per unit particle or more surface area per unit volume for advanced cell therapy. The porosity of the porous particle may be controlled by adjusting manufacturing parameters. For instance, the porosity may be controlled through the use of a porogen.

The generation of pores offers a number of advantages over nonporous structures. These include enhanced nutrient transport and higher surface to area to volume ratio. This 3-dimensional scaffold mimics a bioreactor. This bioreactor is achieved by allowing the porous particles to absorb water, maintain an optimal ion nutrient gradient, and maintain an optimal osmotic pressure which favors cellular growth and cell activation.

Generally speaking, any material that a) can phase separate (is not miscible) with the matrix and b) does not get incorporated into/tethered to the matrix and can be removed after formation of the matrix can be used as a porogen for the synthesis of porous particles. In this way, the porous particle comprises a plurality of micropores, which are formed inherently by monomer polymerization, and a plurality of macropores, which are formed when the porogen is removed from the particle.

In embodiments, the plurality of micropores, which may be formed during polymerization of the monomer within the dispersed phase, may have an average diameter of between about 1 nm and about 20 nm, or between about 2 nm and about 4 nm. In embodiments, the plurality of macropores may have an average diameter of between about 200 nm and about 2 μm.

In some embodiments, macropores of the present disclosure display an average diameter of about 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, or 200 nm, including all ranges and subranges therebetween.

In some embodiments, macropores of the present disclosure display an average diameter of about 0.2 μm, 0.23 μm, 0.26 μm, 0.3 μm, 0.35 μm, 0.4 μm, 0.45 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, or 3.3 μm, including all ranges and subranges therebetween.

In some embodiments, about 1%, 2%, 3%, 4%, 5%, 6%, 7%, 8%, 9%, 1000, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 440%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the volume of the synthetic particles of the present disclosure comprises macropores, including all ranges and subranges therebetween.

In embodiments, the macropore-laden particle may have a diameter substantially similar to the particles described elsewhere herein. For instance, the macropore-laden particle may have a diameter of between about 1 μm and about 25 μm, or between about 2 μm and about 5 μm. In some embodiments, the synthetic particle(s) have an average (mean) diameter of between about 1 μm and about 2 μm, between about 2 μm and about 5 μm, between about 5 μm and about 10 μm, between about 10 μm and about 15 μm, between about 15 μm and about 20 μm, between about 20 μm and about 25 μm, between about 25 μm and about 30 μm, between about 30 μm and about 35 μm, between about 35 μm and about 40 μm, between about 40 μm and about 50 μm, between about 50 μm and about 100 μm, between about 1 μm and about 5 μm, between about 2 μm and about 10 μm, between about 5 μm and about 15 μm, between about 10 μm and about 20 μm, between about 15 μm and about 25 μm, between about 20 μm and about 30 μm, between about 25 μm and about 35 μm, between about 30 μm and about 40 μm, between about 35 μm and about 50 μm, between about 40 μm and about 100 μm, between about 1 μm and about 10 μm, between about 2 μm and about 15 μm, between about 5 μm and about 20 μm, between about 10 μm and about 25 μm, between about 15 μm and about 30 μm, between about 20 μm and about 35 μm, between about 25 μm and about 40 μm, between about 30 μm and about 50 μm, between about 35 μm and about 100 μm, between about 1 μm and about 15 μm, between about 2 μm and about 20 μm, between about 5 μm and about 25 μm, between about 10 μm and about 30 μm, between about 15 μm and about 35 μm, between about 20 μm and about 40 μm, between about 25 μm and about 50 μm, between about 30 μm and about 100 μm, between about 1 μm and about 20 μm, between about 2 μm and about 25 μm, between about 5 μm and about 30 μm, between about 10 μm and about 35 μm, between about 15 μm and about 40 μm, between about 20 μm and about 50 μm, between about 25 μm and about 100 μm, between about 1 μm and about 25 μm, between about 2 μm and about 30 μm, between about 5 μm and about 35 μm, between about 10 μm and about 40 μm, between about 15 μm and about 50 μm, between about 20 μm and about 100 μm, between about 1 μm and about 30 μm, between about 2 μm and about 35 μm, between about 5 μm and about 40 μm, between about 10 μm and about 50 μm, between about 15 μm and about 100 μm, between about 1 μm and about 35 μm, between about 2 μm and about 40 μm, between about 5 μm and about 50 μm, between about 10 μm and about 100 μm, between about 1 μm and about 40 μm, between about 2 μm and about 50 μm, between about 5 μm and about 100 μm, between about 1 μm and about 50 μm, between about 2 μm and about 100 μm, or between about 1 μm and about 100 μm. In some embodiments, the synthetic particle(s) have an average (mean) diameter of between about 1 μm and about 40 μm, between about 10 μm and about 30 μm, between about 15 μm and about 25 μm, or about 20 μm.

In some embodiments, the particles and macropores of the present disclosure are roughly spherical. In some embodiments, diameter of the particles and macropores is based on the longest diameter of said spherical shape.

Moreover, similar to the particles described earlier, the macropore-laden particles may exhibit a Young's modulus of between about 0.2 kPa and about 400 kPa.

In some embodiments, the present disclosure provides methods of producing particles comprising a dispersed monomer phase and a continuous suspension phase, such as oil. Embodiments of these methods recite the presence of a porogen mixed with the monomer phase. In some embodiments, porogens may be immiscible within the monomer, and thus may be said to form a further dispersed phase within the monomer phase (i.e., where porogen may be considered the dispersed phase and the monomer phase would be considered a continuous phase). These embodiments could be described as an emulsion within an emulsion. For the purposes of this disclosure however, the monomer phase is referred to as the dispersed phase, regardless of whether it also includes porogens. The continuous phase refers to the suspension (e.g., oil) phase.

In embodiments, the monomer to be polymerized may be within a first phase and the porogen may be within a second phase.

In embodiments, the porogen may be one or more of a porogen polymer, a water-soluble polymer, a salt, carbon black, a biodegradable polymer, a degradable polymer, seaweed polysaccharides, and a paraffin wax. In some embodiments, the salt comprises one or more of sodium chloride, ammonium bicarbonate, lithium chloride, zinc chloride, silicon dioxide, calcium carbonate, and any combination thereof. For example, calcium carbonate particles can phase separate in particle and get washed away with a low pH buffer. In some embodiments, the porogen polymer comprises one or more of polyethylene glycol, poly(vinylpyrrolidone), polyvinyl alcohol, and any combination thereof. For instance, the porogen polymer may include polymers that are water-soluble but also gel matrix polymer immiscible may also be used.

In embodiments, the porogen polymer can have a linear, branched, hyperbranched, or a bottlebrush structure. In some embodiments, the porogen polymer may comprise polymeric particles that become water-soluble after a stimulus is applied. For example, particles with a degradable crosslinker (e.g. N,N′-Bis(acryloyl)cystamine) can be embedded into particles and then degraded with a cleaving agent. (e.g. reducing agent for N,N′-Bis(acryloyl)cystamine).

In embodiments, creating a porous structure increases the surface area of the particle.

Porous structures can be created on the particles where biomolecules may be conjugated and remain accessible to interactions with antibodies or in inverse, where conjugated antibodies can interact with their antigens on cells. In some embodiments, the porous structures allow for conjugation of a large number of biomolecules (e.g., greater than 100,000, or greater than 1,000,000). All attachment chemistries known to those skilled in the art and/or disclosed in the present disclosure can be used with or incorporated into this technique.

Porogens can also be used to increase the diffusion coefficient of large molecules (such as DNA, proteins, etc.) within particles, or to increase cell affinity of particles for tissue engineering purposes.

Moreover, the side scatter properties of porous particles may more closely match the optical properties of living cells.

The percentage of the material forming the particle, the molecular weight of the porogen and the % concentration of the porogen added can be adjusted to achieve a desired porosity.

In some embodiments, the particles of the present disclosure can be further modified by varying the size of the microsphere (i.e., particle) produced. Size can be controlled by flow rates and/or pressure of the aqueous and oil phase during the microfluidic droplet generation process, as discussed in other portions of this disclosure.

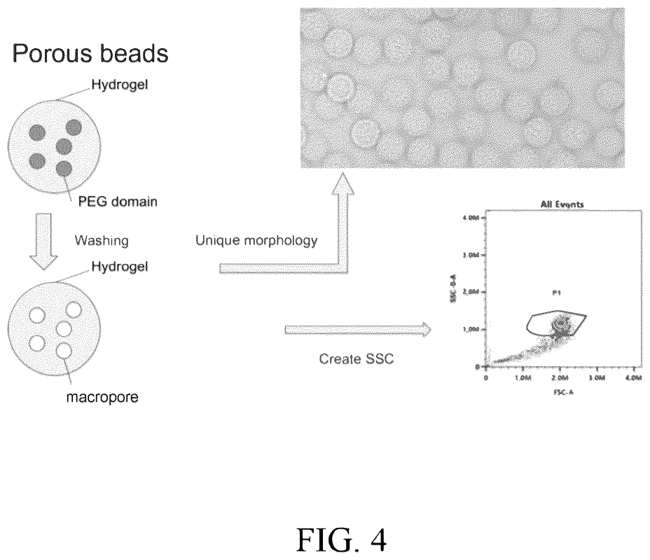

FIG. 4 provides a high-level flow diagram of formation of porous particles, including polymerization of a dispersed phase into a particle, encapsulation of PEG domains therein, and washing of the particle to remove the PEG domains to form macropores. In embodiments, the PEG domains may alternatively, or additionally, be removed by leaching. Unlike washing, which may refer to a solute that is readily dissolvable, leaching may be appropriate when the solute requires more time to dissolve and thus to be removed from the material.

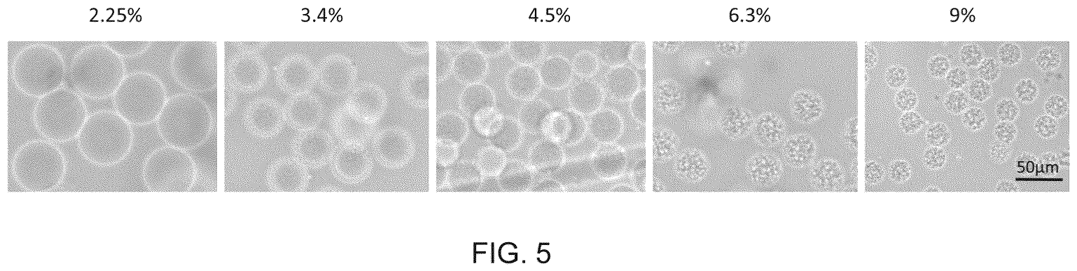

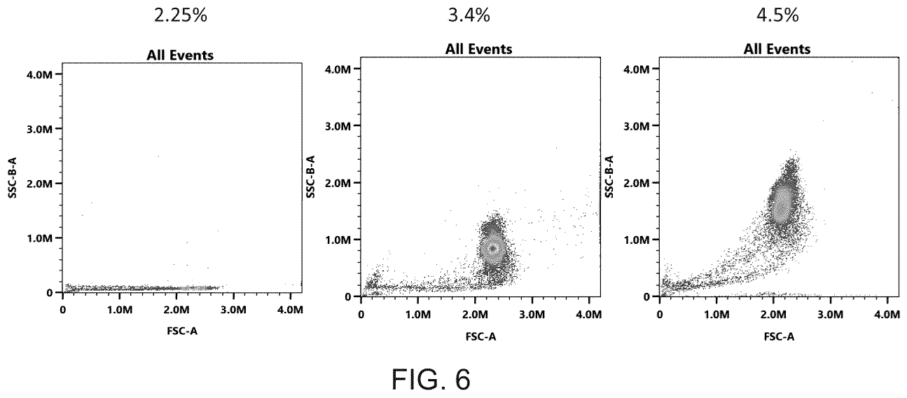

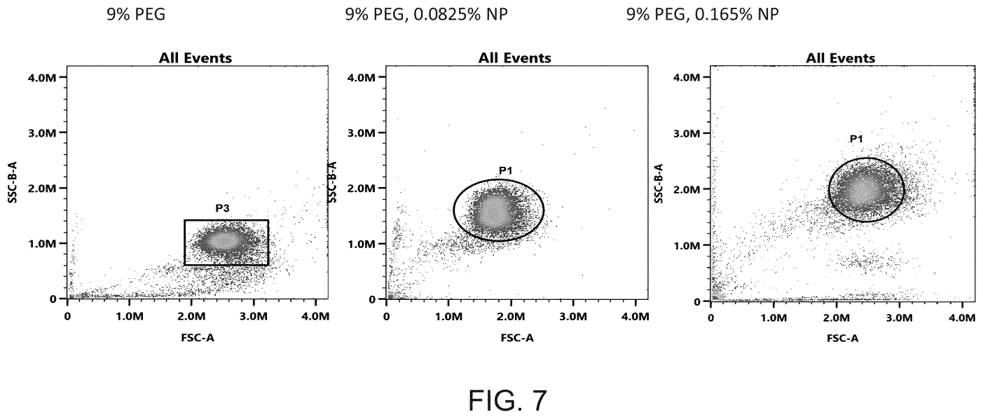

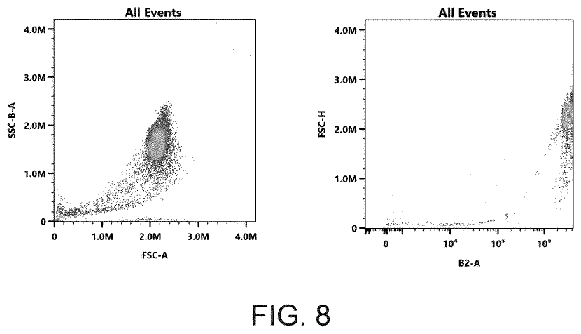

A microscopic image of the porous particles is shown at top right and a side scatter plot is shown at bottom left. FIG. 5 provides a series of microscopic images of porous particles formed with varying levels of PEG, increasing in concentration from left to right. FIG. 6 demonstrates the ability to modify PEG concentrations used during formation to modify side scatter profiles of the resulting porous particle. In some embodiments, nanoparticles can be used in conjunction with porous particles. FIG. 7 demonstrates the ability to modify nanoparticle concentrations within the porous particles to mimic organelles in a target cell. FIG. 8 demonstrates the ability to conjugate fluorophores to the porous particles. Thus, particle porosity is compatible with other described methods of manufacture and modifications of the synthetic particles of the present disclosure.

### PEG as the Porogen

In some embodiments, polyethylene glycol (PEG), which is water-soluble, may be used as the porogen. PEG is immiscible with polyacrylamide.

In some embodiments, inert, linear PEG polymer can be introduced as a porogen into the aqueous or water phase of our microfluidic synthesis of particles. During the curing process, the linear PEG polymers, immiscible with the gel matrix polymer (poly acrylamide in this case), become phase separated with the gel matrix and form its own domains, spatially excluding polyacrylamide particles. After synthesis, the beads are washed with water where the PEG polymers are removed from the matrix. This leaves hollow pores within the particles. These pores create more water/particle interface. The porous particles may also have unique sponge-like morphology that can be observed with microscopy and also useful as cell control for imaging cytometry or any imaging-based cell characterization techniques.

In some embodiments, addition of polyethylene glycol (PEG) to the matrix during synthesis creates pores in the particles that can scatter incident light due to phase transitions between the matrix and the pores containing.

In some embodiments, addition of PEG as a porogen can increase biomolecule binding capacity of the particles by creating a porous surface with increased surface area for the binding of biomolecules.

PEG may not remain in beads once they are washed as the polymer can escape from the particle matrix through surface pores in most formulations.

The percentage of the material forming the particle, the molecular weight of the PEG, and the % concentration of the PEG can be adjusted to achieve a desired porosity. Table 5 shows previously characterized hydrodynamic radius of various PEG polymer molecular weights, and thus the minimum implied pore size introduced by their inclusion in particles, as an example of a porogen polymer used within the particles of the present disclosure.

TABLE 5

| Molecular Weight (kDA) | Hydrodynamic Radius (nm) |
|---|---|
| PEG 200 | 0.49 |
| PEG 400 | 0.65 |
| PEG 1000 | 0.93 |
| PEG 4000 | 1.60 |
| PEG 10,000 | 2.29 |
| PEG 20,000 | 3.01 |
| PEG 40,000 | 3.95 |

In some embodiments, polyethylene glycol (PEG) provides an inert, pore-forming agent that can be used in the aqueous dispersion phase during microfluidic droplet generation. Adding PEG solution during the preparation of raw droplets, followed by removal after polymerization, allows cavities and tunnels to be irreversibly introduced into the matrix of the particle. Adjusting the initial PEG concentration added during the preparation of the raw droplets (e.g., within the dispersed phase) impacts pore size and distribution. In some embodiments, varying the PEG concentration introduced to the particle formulation determines a number of pores per unit volume of the resulting particle matrix. For instance, the PEG concentration within the dispersed phase may be between about 1% w/v and about 99% w/v. For instance, the PEG concentration may be at least about 1%, at least about 2%, at least about 4%, at least about 6%, at least about 8%, at least about 10%, at least about 12%, at least about 14%, at least about 16%, at least about 18%, at least about 20%, at least about 25%, at least about 30%, at least about 35%, or at least about 40% w/v, including all ranges and subranges therebetween. In some embodiments, the PEG concentration introduced during preparation of the particles may be about 9% w/v. In some embodiments, the PEG concentration introduced during preparation of the particles may be about 2.25%, about 3.4%, or about 4.5% w/v, including all ranges and subranges therebetween. In some embodiments, the PEG concentration within the dispersed phase may be between about 1% v/v and about 99% v/v. In embodiments, the PEG solution comprises a variable concentration of PEG 8000.

### Polymerization and Functionalization of Particles

In some embodiments, the particles provided herein are synthesized by polymerizing one or more of the monomers of the present disclosure. The synthesis is carried out to form individual particles. In some embodiments, the monomeric material (monomer) is polymerized to form a homopolymer. In some embodiments, copolymers of different monomeric units (i.e., co-monomers) are synthesized and used in the methods provided herein. In some embodiments, the monomer or co-monomers used in the methods and compositions described herein is a bifunctional monomer or includes a bifunctional monomer (where co-monomers are employed). The use of bifunctional monomers allows for the further derivatization of particles, e.g., with biomolecules, cell surface markers or epitope binding fragments thereof, or a combination thereof. In some embodiments, the particle is synthesized in the presence of a crosslinker. In some embodiments, the particle is synthesized in the presence of a polymerization initiator.

The amount of monomer can be varied by the user, for example to obtain a particular optical property that is substantially similar to that of a target cell. In some embodiments, the monomeric component(s) (i.e., monomer, co-monomer, bifunctional monomer, or a combination thereof, for example, bis/acrylamide in various crosslinking ratios, allyl amine or other co-monomers which provide chemical functionality for secondary labeling/conjugation, or alginate) is present at about 10 percent by weight to about 95 percent weight of the particle. In some embodiments, the monomeric component(s) is present at about 15 percent by weight to about 90 percent weight of the particle, or about 20 percent by weight to about 90 percent weight of the particle.

Examples of various monomers and cross-linking chemistries available for use with the present disclosure are provided in the Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf), the disclosure of which is incorporated by reference in its entirety for all purposes. For example, hydrazine (e.g., with an NHS ester compound) or EDC coupling reactions (e.g., with a maleimide compound) can be used to construct the particles of the disclosure.

In some embodiments, a monomer for use with the particles provided herein is lactic acid, glycolic acid, acrylic acid, 1-hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, a derivatized version thereof, or a combination thereof. In some embodiments, the polymer may be degradable. For instance, the polymer may be a polyester based on polylactide (PLA), polyglycolide (PGA), polycaprolactone, poly(lactic-co-glycolic) acid (PLGA), or their copolymers. Other biodegradable polymers may be used.

In some embodiments, one or more of the following monomers is used herein to form a particle of the present disclosure: 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, hydroxybutyl methacrylate, or a combination thereof.

In some embodiments, one or more of the following monomers is used herein to form a tunable particle: phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzyl acrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, N-phenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, as described in U.S. Pat. No. 6,657,030, which is incorporated by reference in its entirety herein for all purposes.

Both synthetic monomers and bio-monomers can be used in the particles provided herein, to form synthetic particles. In some embodiments, the synthetic particles may comprise a chemical component and a bio-component (e.g., peptide, protein, monosaccharide, disaccharide, polysaccharide, primary amines sulfhydryls, carbonyls, carbohydrates, carboxylic acids present on a biomolecule). For example, proteins, peptides or carbohydrates can be used as individual monomers to form a particle that includes or does not include a synthetic monomer (or polymer) and in combination with chemically compatible co-monomers and crosslinking chemistries (see, e.g., the Thermo Scientific Crosslinking Technical Handbook entitled “Easy molecular bonding crosslinking technology,” available at tools. lifetechnologies.com/content/sfs/brochures/1602163-Crosslinking-Reagents-Handbook.pdf, the disclosure of which is incorporated by reference in its entirety for all purposes). Compatible crosslinking chemistries include, but are not limited to, amines, carboxyls, and other reactive chemical side groups. Representative reactive groups amenable for use in the particles and monomers described herein are provided in Table 1, below.

TABLE 1
Crosslinker reactive groups amenable for bio-monomer conjugation

| | Target functional | |
|---|---|---|
| Reactivity class | group | Reactive chemical group |
| Amine reactive | —NH₂ | NHS ester |
| | | Imidoester |
| | | Penafluorophenyl ester |
| | | Hydroxymethyl phosphine |
| Carboxyl-to-amine reactive | —COOH | Carbodiimide (e.g., EDC) |
| Sulfhydryl-reactive | —SH | Maleimide |
| | | Haloacetyl (bromo- or iodo-) |
| | | Pyridylisulfide |
| | | Thiosulfonate |
| | | Vinylsulfonate |
| Aldehyde-reactive (oxidized sugars, | —CHO | Hydrazine |
| carbonyls) | | Alkoxyamine |
| Photo-reactive (e.g., nonselective, | Random | Diazirine |
| random insertion) | | Aryl azide |
| Hydroxyl (nonaqueous)-reactive | —OH | Isocyanate |
| Azide-reactive | —N3 | Phosphine |

In general, any form of polymerization chemistry/methods known by those skilled in the art can be employed to form polymers. In some embodiments, polymerization can be catalyzed by ultraviolet light-induced radical formation and reaction progression. In other embodiments, a particle of the disclosure is produced by the polymerization of acrylamide or the polymerization of acrylate. For example, the acrylamide in some embodiments is a polymerizable carbohydrate derivatized acrylamide as described in U.S. Pat. No. 6,107,365, the disclosure of which is incorporated by reference in its entirety for all purposes. As described therein and known to those of ordinary skill in the art, specific attachment of acrylamide groups to sugars is readily adapted to a range of monosaccharides and higher order polysaccharides, e.g., synthetic polysaccharides or polysaccharides derived from natural sources, such as glycoproteins found in serum or tissues.

In some embodiments, an acrylate-functionalized poly(ethylene) glycol monomer is used as a monomer. For example, the PEG In some embodiments is an acrylate or acrylamide functionalized PEG.

In some embodiments, a particle comprises a monofunctional monomer polymerized with at least one bifunctional monomer. One example includes, but is not limited to, the formation of poly-acrylamide polymers using acrylamide and bis-acrylamide (a bifunctional monomer). In some embodiments, a particle provided herein comprises a bifunctional monomer polymerized with a second bifunctional monomer. One example includes, but is not limited to, the formation of polymers with mixed composition containing compatible chemistries such as acrylamide, bis-acrylamide, and bis-acrylamide structural congeners containing a wide range of additional chemistries. The range of chemically compatible monomers, bifunctional monomers, and mixed compositions is obvious to those skilled in the art and follows chemical reactivity principles know to those skilled in the art. See, e.g., the Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf) and the Polyacrylamide Emulsions Handbook (SNF Floerger, available at snf.com.au/downloads/Emulsion_Handbook_E.pdf), the disclosure of each of which is incorporated by reference in its entirety for all purposes.

In some embodiments, a particle provided herein comprises a polymerizable monofunctional monomer and is a monofunctional acrylic monomer. Non-limiting examples of monofunctional acrylic monomers for use herein are acrylamide; methacrylamide; N-alkylacrylamides such as N-ethylacrylamide, N-isopropylacrylamide or N-tert-butylacrylamide; N-alkylmethacrylamides such as N-ethylmethacrylamide or N-isopropylmethacrylamide; N,N-dialkylacrylamides such as N,N-dimethylacrylamide and N, N-diethyl-acrylamide; N-[(dialkylamino)alkyl]-acrylamides such as N-[3dimethylamino)-propyl]-acrylamide or N-[3-(diethylamino)propyl]-acrylamide; N-[(dialkylamino)alkyl]-methacrylamides such as N-[3-dimethylamino)propyl]methacrylamide or N-[3-(diethylamino) propyl]methacrylamide; (dialkylamino)alkyl acrylates such as 2-(dimethylamino)ethyl acrylate, 2-(dimethylamino)propyl acrylate, or 2-(diethylamino)ethyl acrylates; and (dialkylamino) alkyl methacrylates such as 2-(dimethylamino) ethyl methacrylate.

A bifunctional monomer is any monomer that can polymerize with a monofunctional monomer of the disclosure to form a particle as described herein that further contains a second functional group that can participate in a second reaction, e.g., conjugation of a fluorophore, cell surface receptor (or domain thereof), or immune co-stimulatory biomolecule.

In some embodiments, a bifunctional monomer is selected from the group consisting of allyl amine, allyl alcohol, allyl isothiocyanate, allyl chloride, and allyl maleimide.

A bifunctional monomer can be a bifunctional acrylic monomer. Non-limiting examples of bifunctional acrylic monomers are N,N′-methylenebisacrylamide, N,N′-methylene bismethacrylamide, N,N′-ethylene bisacrylamide, N,N′-ethylene bismethacrylamide, N,N′-propylenebisacrylamide, and N,N′-(1,2-dihydroxyethylene) bisacrylamide.

Higher order branched chain and linear co-monomers can be substituted in the polymer mix to adjust the refractive index while maintaining polymer density, as described in U.S. Pat. No. 6,657,030, which is incorporated herein by reference in its entirety for all purposes.

In some embodiments, a particle comprises a molecule that modulates the optical properties of the particle.

In some embodiments, the biomonomer is functionalized with acrylamide or acrylate. For example, in some embodiments, the polymerizable acrylamide functionalized biomolecule is an acrylamide or acrylate functionalized protein (for example, an acrylamide functionalized collagen or functionalized collagen domain), an acrylamide or acrylate functionalized peptide, or an acrylamide or acrylate functionalized monosaccharide, disaccharide or polysaccharide.

Any monosaccharide, disaccharide or polysaccharide (functionalized or otherwise) can be used. In some embodiments, an acrylamide or acrylate functionalized monosaccharide, disaccharide or polysaccharide is used as a polymerizable monomer. In some embodiments, a structural polysaccharide is used as a polymerizable monomer. In some embodiments, the structural polysaccharide is an arabinoxylan, cellulose, chitin or a pectin. In some embodiments, alginic acid (alginate) is used as a polymerizable monomer. In yet another embodiment, a glycosaminoglycan (GAG) is used as a polymerizable monomer in the particles provided herein. In some embodiments, the GAG is chondroitin sulfate, dermatan sulfate, keratin sulfate, heparin, heparin sulfate or hyaluronic acid (also referred to in the art as hyaluron or hyaluronate) is used as a polymerizable monomer. The additional range of compatible biomonomers and their reactive chemistries are known be individuals skilled in the art and follow general chemical reactivity principles.

An additional range of biocompatible monomers that can be incorporated are known in the art, see, for example the non-degradable biocompatible monomers disclosed in Shastri (2003). Current Pharmaceutical Biotechnology 4, pp. 331-337, incorporated by reference herein in its entirety for all purposes. Other monomers are provided in de Moraes Porto (2012). Polymer Biocompatibility, Polymerization, Dr. Ailton De Souza Gomes (Ed.), ISBN: 978-953-51-0745-3; InTech, DOI: 10.5772/47786; Heller et al. (2010). Journal of Polymer Science Part A: Polymer Chemistry 49, pp. 650-661; Final Report for Biocompatible Materials (2004), The Board of the Biocompatible Materials and the Molecular Engineering in Polymer Science programmes, ISBN 91-631-4985-0, the disclosure of each of which is hereby incorporated by reference in its entirety for all purposes.

Biocompatible monomers for use with the particles described herein include in some embodiments, ethylene glycol dimethacrylate (EGDMA), 2-hydroxyethyl methacrylate (HEMA), methyl methacrylate (MMA), methacryloxymethyltrimethylsilane (TMS-MA), N-vinyl-2-pyrrolidon (N-VP), styrene, or a combination thereof.

Naturally occurring particles useful in this disclosure include various polysaccharides available from natural sources such as plants, algae, fungi, yeasts, marine invertebrates and arthropods. Non-limiting examples include agarose, dextrans, chitin, cellulose-based compounds, starch, derivatized starch, and the like. These generally will have repeating glucose units as a major portion of the polysaccharide backbone. Cross-linking chemistries for such polysaccharides are known in the art, see for example Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf).

Hyaluronan in some embodiments is used as a monomer (either as a single monomer or as a co-monomer). In some embodiments, hyaluronan is functionalized, for example with acrylate or acrylamide. Hyaluronan is a high molecular weight GAG composed of disaccharide repeating units of N-acetylglucosamine and glucuronic acid linked together through alternating β-1,4 and β-1,3 glycosidic bonds. In the human body, hyaluronate is found in several soft connective tissues, including skin, umbilical cord, synovial fluid, and vitreous humor. Accordingly, in some embodiments, where one or more properties of a skin cell, umbilical cord cell or vitreous humor cell is desired to be mimicked, in some embodiments, hyaluronan is used as a monomer. Methods for fabricating particles are described in Xu et al. (2012). Soft Matter. 8, pp. 3280-3294, the disclosure of which is incorporated herein in its entirety for all purposes. As described therein, hyaluronan can be derivatized with various reactive handles depending on the desired cross-linking chemistry and other monomers used to form a particle.

In some embodiments, chitosan, a linear polysaccharide composed of randomly distributed β-(1-4)-linked D-glucosamine (deacetylated unit) and N-acetyl-D-glucosamine (acetylated unit), is used as a monomer (either as a single monomer or as a co-monomer).

Other polysaccharides for use as a monomer or co-monomer include but are not limited to, agar, agarose, alginic acid, alguronic acid, alpha glucan, amylopectin, amylose, arabinoxylan, beta-glucan, callose, capsulan, carrageenan polysaccharides (e.g., kappa, iota or lambda class), cellodextrin, cellulin, cellulose, chitin, chitosan, chrysolaminarin, curdlan, cyclodextrin, alpha-cyclodextrin, dextrin, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, zymosan, or a combination thereof. As described throughout, depending on the desired cross-linking chemistry and/or additional co-monomers employed in the particle, the polysaccharide can be further functionalized. For example, one or more of the polysaccharides described herein in some embodiments is functionalized with acrylate or acrylamide.

In some embodiments, an individual particle or a plurality thereof comprises a peptide, protein, a protein domain, or a combination thereof as a monomer or plurality thereof. In some embodiments, the protein is a structural protein, or a domain thereof, for example, such as silk, elastin, titin or collagen, or a domain thereof. In some embodiments, the protein is an extracellular matrix (ECM) component (e.g., collagen, elastin, proteoglycan, fibrin, lysine, fibronectin). In some embodiments, the structural protein is collagen. In some embodiments, the collagen is collagen type I, collagen type II or collagen type III or a combination thereof. In some embodiments, the monomer comprises a proteoglycan. In some embodiments, the proteoglycan is decorin, biglycan, testican, bikunin, fibromodulin, lumican, or a domain thereof.

In some embodiments, an acrylate-functionalized structural protein monomer is used as a component of the particle provided herein (e.g., an acrylate functionalized protein or protein domain, for example, silk, elastin, titin, collagen, proteoglycan, or a functionalized domain thereof). In some embodiments, the acrylate functionalized structural protein monomer comprises a proteoglycan, e.g., decorin, biglycan, testican, bikunin, fibromodulin, lumican, or a domain thereof.

In some embodiments, PEG monomers and oligopeptides can be that mimic extracellular matrix proteins are used in the particles provided herein, for example, with vinyl sulfone-functionalized multi arm PEG, integrin binding peptides and bis-cysteine matrix metalloproteinase peptides as described by Lutolf et al. (2003). Proc. Nat. Acad. Sci. U.S.A. 100, 5413-5418, incorporated by reference in its entirety for all purposes. In some embodiments, particles are formed by a Michael-type addition reaction between the di-thiolated oligopeptides and vinyl sulfone groups on the PEG. The range of additional compatible chemistries that can be incorporated here are apparent to those skilled in the art and follow general chemical reactivity principles, see for example Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf).

Other bioactive domains in natural proteins can also be used as a monomer or portion thereof. For example, a cell-adhesive integrin binding domain, a controlled release affinity binding domain or a transglutaminase cross-linking domain can be used in the particles provided herein. Details for producing such particles can be found in Martino et al. (2009). Biomaterials 30, 1089; Martino et al. (2011). Sci. Trans. Med. 3, 100ra89; Hu and Messersmith (2003). J Am. Chem. Soc. 125, 14298, each of which is incorporated by reference in its entirety for all purposes.

In some embodiments, recombinant DNA methods are used to create proteins, designed to gel in response to changes in pH or temperature, for example, by the methods described by Petka et al. (1998). Science 281, pp. 389-392, incorporated by reference in its entirety for all purposes. Briefly, the proteins consist of terminal leucine zipper domains flanking a water-soluble polyelectrolyte segment. In near-neutral aqueous solutions, coiled-coil aggregates of the terminal domains form a three-dimensional polymer network.

Common crosslinking agents that can be used to crosslink the particles provided herein include but are not limited to ethylene glycol dimethacrylate (EGDMA), tetra ethylene glycol dimethacrylate, and N,N′-15 methylenebisacrylamide. The range of additional crosslinking chemistries which can be used will be apparent to those skilled in the art and follow general chemical reactivity principles, see for example Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf).

In some embodiments, polymerization of a monomer is initiated by a persulfate or an equivalent initiator that catalyzes radical formation. The range of compatible initiators are known to those skilled in the art and follow general chemical reactivity principles, see for example Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf). The persulfate can be any water-soluble persulfate. Non-limiting examples of water-soluble persulfates are ammonium persulfate and alkali metal persulfates. Alkali metals include lithium, sodium and potassium. In some embodiments, the persulfate is ammonium persulfate or potassium persulfate. In some embodiments, polymerization of the monomer provided herein is initiated by ammonium persulfate.

Polymerization of a monomer can be accelerated by an accelerant which can catalyze the formation of polymerization-labile chemical side groups. The range of possible accelerants is known to those skilled in the art and follow general chemical reactivity principles. See for example Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf). In some embodiments, the accelerant is a tertiary amine. The tertiary amine can be any water-soluble tertiary amine. In some embodiments, an accelerant is used in the polymerization reaction and is 3-(dimethylamino) propionitrile, or N,N,N′,N′tetramethylethylenediamine (TEMED). In some embodiments, an accelerant is used in the polymerization reaction and is azobis(isobutyronitrile) (AIBN).

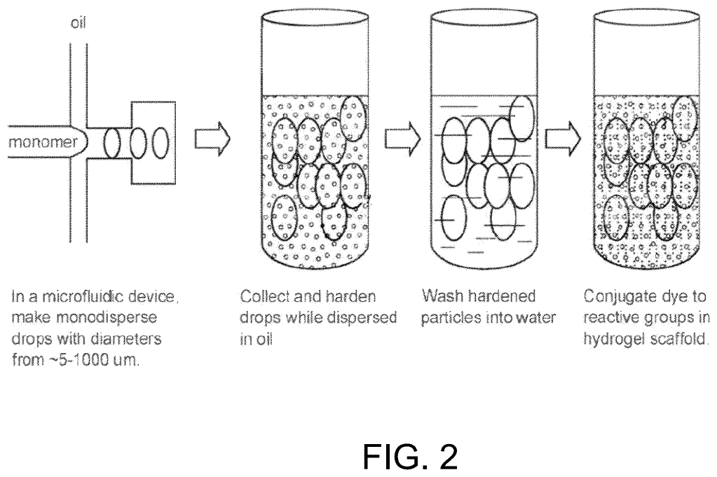

As discussed above, the particle for use in the compositions and methods described herein can include any of the monomeric units and crosslinkers as described herein, and in some embodiments are produced as particles by polymerizing droplets (see, e.g., FIG. 2). Microfluidic methods of producing a plurality of droplets, including fluidic and rigidified droplets, are known to those of ordinary skill in the art, and described in US Patent Publication No. 2011/0218123 and U.S. Pat. No. 7,294,503, each incorporated herein by reference in its entirety for all purposes. Such methods provide for a plurality of droplets containing a first fluid (e.g., dispersed phase) and being substantially surrounded by a second fluid (e.g., a continuous phase), where the first fluid and the second fluid are substantially immiscible (e.g., droplets containing an aqueous-based liquid being substantially surrounded by an oil-based liquid).

A plurality of fluidic droplets (e.g., prepared using a microfluidic device) may be polydisperse (e.g., having a range of different sizes), or in some cases, the fluidic droplets may be monodisperse or substantially monodisperse, e.g., having a homogenous distribution of diameters, for instance, such that no more than about 10%, about 5%, about 3%, about 1%, about 0.03%, or about 0.01% of the droplets have a diameter that is about 10%, about 5%, about 3%, or about T % greater than the average diameter. The average diameter of a population of droplets, as used herein, refers to the arithmetic average of the diameters of the droplets. Average diameters of the particles can be measured, for example, by light scattering techniques. In some embodiments, average diameters of particles are tailored, for example by varying flow rates of the fluid streams of the first and second fluids within the channel(s) of a microfluidic device, or by varying the volume of the channel(s) of the microfluidic device.

Accordingly, the disclosure provides population of particles comprising a plurality of particles, wherein the population of particles is substantially monodisperse.

The term “microfluidic” refers to a device, apparatus or system including at least one fluid channel having a cross-sectional dimension of less than 1 mm, and a ratio of length to largest cross-sectional dimension perpendicular to the channel of at least about 3:1. A microfluidic device comprising a microfluidic channel is especially well suited to preparing a plurality of monodisperse droplets.

Non-limiting examples of microfluidic systems that may be used with the present disclosure are disclosed in U.S. Patent Application Publication No. 2006/0163385; U.S. Patent Application Publication No. 2005/0172476; U.S. Patent Application Publication No. 2007/000342; International Patent Application Publication No. WO 2006/096571; U.S. Patent Application Publication No. 2007/0054119; U.S. Pat. No. 7,776,927; and International Patent Application Publication No. WO 2006/078841, each incorporated herein by reference in its entirety for all purposes.

Droplet size is related to microfluidic channel size. The microfluidic channel may be of any size, for example, having a largest dimension perpendicular to fluid flow of less than about 5 mm or 2 mm, or less than about 1 mm, or less than about 500 μm, less than about 200 μm, less than about 100 μm, less than about 60 μm, less than about 50 μm, less than about 40 μm, less than about 30 μm, less than about 25 μm, less than about 10 μm, less than about 3 μm, less than about 1 μm, less than about 300 nm, less than about 100 nm, less than about 30 nm, or less than about 10 nm.

Droplet size can be tuned by adjusting the relative flow rates. In some embodiments, drop diameters are equivalent to the width of the channel, or within about 10%, 15%, 20%, 30%, 40%, 50%, 60%, 70%, 80%, 90%, or 100% the width of the channel.

In some embodiments, the dimensions of a particle of the disclosure are substantially similar to the droplet from which it was formed. Therefore, in some embodiments, a particle has a diameter of less than about 1, 2, 5, 10, 15, 20, 25, 30, 35, 40, 45, 50, 60, 70, 80, 90, 100, 120, 150, 200, 250, 300, 350, 400, 450, 500, 600, 800, or 1000 μm in diameter, including all ranges and subranges therebetween. In some embodiments, a particle has a diameter of more than about 1, 2, 5, 10, 15, 20, 25, 30, 35, 40, 45, 50, 60, 70, 80, 90, 100, 120, 150, 200, 250, 300, 350, 400, 450, 500, 600, 800, or 1000 μm in diameter. In some embodiments, a particle has a diameter in the range of 5 μm to 100 μm, including all ranges and subranges therebetween.

In some embodiments, a particle of the disclosure is spherical in shape.

In some embodiments, a particle of the disclosure does not comprise agarose.

In some embodiments, particle manufacturing is carried out by suspension polymerization, which is also referred to in the art as pearl, bead or granular polymerization (see Elbert (2011). Acta Biomater. 7, pp. 31-56, incorporated by reference herein in its entirety for all purposes). In suspension polymerization, the monomer is insoluble in the continuous phase, for example an aqueous monomer solution in a continuous oil phase. In suspension polymerization, polymerization initiation occurs within the monomer-rich droplets and with greater than one radical per droplet at any time. In some embodiments, the monomer phase includes a monomer which can be a bifunctional monomer or a plurality of monomer species (co-monomers, which can be a plurality of bifunctional monomers). In some embodiments, the monomer phase includes an initiator and/or a crosslinking agent.

Emulsion polymerization can also be used to form the particles described herein. In emulsion polymerization, the monomer has poor solubility in the continuous phase, similar to suspension polymerization, however, polymerization initiation occurs outside the monomer droplets (see Elbert (2011). Acta Biomater. 7, pp. 31-56, incorporated by reference herein in its entirety for all purposes). In emulsion polymerization embodiments, the initiator causes chain growth of the monomer (or co-monomers) dissolved in the continuous phase or monomer contained in micelles if surfactants are present.

In some embodiments, particles are formed by precipitation polymerization, for example as described in Elbert (2011). Acta Biomater. 7, pp. 31-56, incorporated by reference herein in its entirety for all purposes. Precipitation polymerization is a technique that takes advantage of the differences in the solubility of monomer and polymer to produce microparticles. Specifically, it is known that larger polymer chains generally have lower solubility than smaller ones. Accordingly, above a specific molecular weight, phase separation may be favored. Precipitation polymerization initially begins as solution polymerizations in a single phase, homogenous system. In some embodiments, shortly after the start of the polymerization, a relatively high concentration of polymer chains is present, favoring phase separation by nucleation. As polymerization proceeds, the concentration of polymer chains is low and existing particles capture the chains before nucleation of new particles can occur. Thus, nucleation of particles occurs only for a brief period of time shortly after the start of the reaction, which, in some embodiments, results in a narrow size distribution of particles. Additional methods include but are not limited to lithographic particle formation (Helgeson et al. (2011). Curr. Opin. Colloid. Interface Sci. 16, pp. 106-117, incorporated by reference herein in its entirety for all purposes), membrane emulsification (e.g., by the microsieve emulsification technology techniques described by Nanomi B.V. (Netherlands)), microchannel emulsification (Sugiura et al. (2002). Languimir 18, pp. 5708-5712, incorporated by reference herein in its entirety) and bulk emulsification (SNF Floerger, available at snf.com.au/downloads/Emulsion_Handbook_E.pdf, incorporated by reference herein in its entirety).

In some embodiments, particles are formed within a microfluidic device having two oil channels that focus on a central stream of aqueous monomer solution. In some embodiments, droplets form at the interface of the two channels and central stream to break off droplets in water-in-oil emulsion. In some embodiments, once droplets are formed, they are stabilized prior to polymerization, for example, by adding a surfactant to the oil phase. However, in some embodiments, droplets are not stabilized prior to polymerization. In some embodiments, polymerization of the monomer is triggered by adding an accelerator (e.g., N,N,N′,N′-tetramethylethylenediamine) to one or both of the oil channels after initial droplets are formed.

The aqueous monomer solution as provided above can include a single monomer species or a plurality of monomer species. The aqueous monomer solution can include co-monomers, a bifunctional monomer, or a combination thereof. In some embodiments, the monomer or plurality of monomers can include a bifunctional monomer, for example, one of the monomers described herein. In some embodiments, co-monomers can be used to modulate forward scatter or side scatter, for example, by adjusting the refractive index of the particle.

In some embodiments, the central stream of aqueous monomer solution comprises a cross-linker, for example, N,N′-bisacrylamide. In some embodiments, the central stream of aqueous monomer solution comprises a cross-linker and an accelerator, in addition to the monomer. In some embodiments, the aqueous monomer solution comprises an initiator, for example an oxidizing agent such as ammonium persulfate.

In some embodiments, forward scatter is modulated by adjusting the refractive index of the gel by adding co-monomers allyl acrylate and allyl methacrylate. Forward scatter can also be modulated with side scattering nanoparticles containing sufficient optical resolution/size/density including, but not limited to, higher density colloidal suspensions of silica and/or PMMA particles. Side scattering of the droplets can be tuned by adding a colloidal suspension of silica nanoparticles and/or PMMA (poly(methyl methacrylate)) particles (˜100 nm) to the central aqueous phase prior to polymerization.

In some embodiments, a bead, plurality of beads, biomolecule, or plurality of biomolecules is embedded (encapsulated) within the particle. In some embodiments, an encapsulated bead or biomolecule is employed to mimic one or more intracellular organelles of a target cell, or a cell after it engulfs a particle. In some embodiments, encapsulating or embedding a bead or biomolecule is accomplished at the time of particle formation. For example, beads can be suspended in the appropriate concentration to allow for an average of one bead to be embedded/encapsulated in a single particle. The bead suspension can be included, for example, within the aqueous solution of monomer. Similarly, a biomolecule or mixture of biomolecules can be incorporated into the aqueous solution of monomer to encapsulate the biomolecule or biomolecules.

In some embodiments, once a particle is formed, for example by the methods described above, it can be further manipulated, for example, by embedding a bead, plurality of beads, biomolecule or plurality of biomolecules within the particle.

Accordingly, in some embodiments of the disclosure, a particle comprising an embedded substance is provided.

In some embodiments, the embedded substance is an embedded molecule, for example a biomolecule. The biomolecule can be a single species or a plurality of different species. For example, a protein, peptide, carbohydrate, nucleic acid or combination thereof can be encapsulated within a particle of the disclosure. Moreover, different nucleic acid molecules (e.g., of varying sequences or nucleic acid type such as genomic DNA, messenger RNA or DNA-RNA hybrids) can be encapsulated by the particle of the disclosure. These can be comprised of any protein or nucleic acid as both forms of biological material contain labile chemical side-groups (or can be modified by commercial vendors (e.g., Integrated DNA Technology chemical side group modifications). Such side-groups are compatible with reaction chemistries commonly found in co-monomer compositions (e.g., acrylate chemistry, NHS-ester, primary amines, copper catalyzed click chemistry (Sharpless)). The range of possible embedded molecules which contain compatible chemistries is understood by those skilled in the art. In some embodiments, embedded molecules can also be attached on particle surfaces, including micro and/or macropore surfaces.

In some embodiments, different subpopulations of particles are fabricated, each with a different concentration of biomolecule. In some embodiments, the biomolecule is a nucleic acid, a protein, an intracellular ion such as calcium acid (or other biomolecule of the user's choosing, for example, calcium). In some embodiments, different subpopulations of particles are fabricated, each with a different concentration of a drug substance. In some embodiments, the drug substance is a biomolecule (i.e., a biologic, antibody or antigen-binding fragment thereof, antibody drug conjugate, protein/enzyme, peptide, non-ribosomal peptide, or related molecule) or a small molecule synthetic drug (e.g., Type I/II/III polyketide, non-ribosomal peptide with bioactive properties, or other small molecule entity as generally classified by those skilled in the art).

In some embodiments, a particle of the disclosure has material modulus properties (e.g., elasticity) more closely resembling that of a target cell as compared to a polystyrene bead of the same diameter.

After the particle is formed, one or more of the particle's surfaces can be functionalized, for example, to mimic one or more optical properties of a target cell or a labeled target cell, or to imbue the particle with immunostimulatory properties. The functionalized particle can also include an embedded bead or substance such as a biomolecule, as described above. In some embodiments, one or more particles are functionalized with one or more fluorescent dyes, one or more cell surface markers/immune co-stimulatory biomolecules (or epitope binding regions thereof), or a combination thereof. In some embodiments, the particle is formed by polymerizing at least one bifunctional monomer and after formation, the particle includes one or more functional groups that can be used for further attachment of a cell surface marker, an epitope binding region of a cell surface marker, a fluorescent dye, or combination thereof. In some embodiments, the free functional group is an amine group, a carboxyl group, a hydroxyl group, or a combination thereof. Depending on the functionalization desired, it is to be understood that multiple bifunctional monomers can be used, for example, to functionalize the particle, for example using different chemistries and with different molecules.

A particle can be functionalized with any fluorescent dye known in the art, including fluorescent dyes listed in The Molecular Probes Handbook-A Guide to Fluorescent Probes and Labeling Technologies, incorporated herein by reference in its entirety for all purposes. Functionalization can be mediated by a compound comprising a free amine group, e.g., allylamine, which can be incorporated into a bifunctional monomer used to form the particle, as discussed herein.

Non-limiting examples of known fluorescent dyes that can be used to functionalize the surface of a particle described herein include: 6-carboxy-4′, 5′-dichloro-2′, 7′-dimethoxyfluorescein succinimidylester; 5-(6)-carboxyeosin; 5-carboxyfluorescein;6 carboxyfluorescein; 5-(6)-carboxyfluorescein; S-carboxyfluorescein-bis-(5-carboxymethoxy-2-nitrobenzyl)ether, 0-alanine-carboxamide, or succinimidyl ester; 5-carboxyfluoresceinsuccinimidyl ester; 6-carboxyfluorescein succinimidyl ester;5-(6)-carboxyfluorescein succinimidyl ester; 5-(4,6-dichlorotriazinyl) amino fluorescein; 2′, 7′-difluoro fluorescein; eosin-5-isothiocyanate; erythrosin5-isothiocyanate; 6-(fluorescein-5-carboxamido) hexanoic acid or succinimidyl ester; 6-(fluorescein-5-(6)-carboxamido)hexanoic acid or succinimidylester; fluorescein-S-EX succinimidyl ester; fluorescein-5-isothiocyanate; fluorescein-6-isothiocyanate; Oregon Green® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; Rhodamine Green™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfone rhodamine or di(succinimidylester); 5-(6)carboxynaphtho fluorescein,5-(6)-carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(6)-carboxyrhodamine6G succinimidyl ester;5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(6)-carboxytetramethylrhodamine;5-carboxytetramethylrhodamine succinimidyl ester; 6-carboxytetramethylrhodaminesuccinimidyl ester; 5-(6)-carboxytetramethylrhodamine succinimidyl ester;6-carboxy-X-rhodamine; 5-carboxy-X-rhodamine succinimidyl ester;6-carboxy-Xrhodamine succinimidyl ester; 5-(6)-carboxy-X-rhodaminesuccinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21 carboxylic acid or succinimidyl ester; QSY® 7 carboxylic acid or succinimidyl ester; Rhodamine Red™-X succinimidyl ester; 6-(tetramethylrhodamine-5-(6)-carboxamido) hexanoic acid; succinimidyl ester; tetramethylrhodamine-5-isothiocyanate;tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; and X-rhodamine-5-(6) isothiocyanate.

Other examples of fluorescent dyes for use with the particles described herein include, but are not limited to, BODIPY® dyes commercially available from Invitrogen, including, but not limited to BODIPY® FL; BODIPY® TMR STP ester; BODIPY® TR-X STP ester; BODIPY® 630/650-X STPester; BODIPY® 650/665-X STP ester; 6-dibromo-4,4-difluoro-5, 7-dimethyl-4-bora-3a,4a-diaza-s-indacene-3-propionic acid or succinimidyl ester; 4,4-difluoro-4-bora-3a,4a-diaza-s-indacene-3,5-dipropionic acid;4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid or succinimidyl ester; 4,4-difluoro-5,7-dimethyl-4-bora-3a, 4a-diaza-s-indacene-3propionic acid; 4,4-difluoro-5,7-dimethyl-4-bora-3a,4a-diaza-s-indacene-3-propionicacid succinimidyl ester; 4, 4difluoro-5,7-dimefhyl-4-bora-3a,4a-diaza-s-indacene-3propionic acid; sulfosuccinimidyl ester, or sodium salt; 6-((4,4-difluoro-5, 7-dimethyl-4-bora-3a,4a-diaza-s-indacene-3propionyl)amino)hexanoic acid; 6-((4,4-difluoro-5, 7-dimethyl-4-bora-3a,4a-diaza-s-indacene-3-propionyl)amino) hexanoic acid or succinimidyl ester; N-(4, 4-difluoro-5,7-dimethyl-4-bora-3a,4a-diaza-s-indacene-3-propionyl) cysteic acid, succinimidyl ester, or triethylammonium salt; 6-4,4-difluoro-1,3-dimethyl-5-(4-methoxyphenyl)-4-bora-3a,4a-4,4-difluoro-5,7-diphenyl-4-bora-3a,4a-diaza-s-indacene-3-propionic acid; 4,4-difluoro-5,7-diphenyl-4-bora3a, 4a-diaza-s-indacene-3-propionic acid, or succinimidyl ester; 4,4-difluoro-5-phenyl-4-bora-3a,4a-diaza-s-indacene-3-propionic acid, or succinimidyl ester; 6-((4,4-difluoro-5-phenyl-4-bora-3a,4a-diaza-s-indacene-3-propionyl)amino) hexanoic acid or succinimidyl ester; 4,4-difluoro-5-(4-phenyl-1,3butadienyl)-4-bora-3a,4a-diaza-s-indacene-3-propionicacid or succinimidyl ester; 4,4-difluoro-5-(2-pyrrolyl)-4-bora-3a,4a-diaza-s-indacene-3-propionic acid or succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester;4,4-difluoro-5-styryl-4-bora-3 a, 4a-diaza-s-indacene-3-propionic acid; 4, 4-difluoro-5-styryl-4-bora-3a,4a-diaza-s-indacene-3-propionic acid or succinimidyl ester; 4,4-difluoro-1,3,5,7-tetramethyl-4-bora-3a,4a-diaza-s-indacene-8-propionic acid; 4,4-difluoro-1,3,5,7-tetramethyl-4bora-3a,4a-diaza-s-indacene-8-propionic acid or succinimidyl ester; 4,4-difluoro-5-(2-thienyl)-4-bora-3a,4a-diaza-s-indacene-3-propionic acid or succinimidyl ester; 6-(((4-(4,4-difluoro-5-(2-thienyl)-4-bora-3a,4a-diaza-s-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl) aminohexanoic acid or succinimidyl ester

Fluorescent dyes for derivatization of the surface of one or more particles include, but are not limited to, Alexa Fluor® dyes commercially available from Invitrogen, including but not limited to Alexa Fluor® 350 carboxylic acid; Alexa Fluor® 430 carboxylic acid; Alexa Fluor® 488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor® 594 carboxylic acid; Alexa Fluor® 633 carboxylic acid; Alexa Fluor® 647 carboxylic acid; Alexa Fluor® 660 carboxylic acid; and Alexa Fluor® 680 carboxylic acid. In some embodiments, fluorescent dyes for use with the particles and methods described herein include cyanine dyes commercially available from Amersham-Pharmacia Biotech, including, but not limited to Cy3 NHS ester; Cy5 NHS ester; Cy5.5 NHSester; and Cy7 NHS ester.

It is within the ordinary skill in the art to select a suitable dye or dyes based on the desired spectral excitation and emission properties of the particle.

In some embodiments, particles are functionalized with one or more biomolecules, such as cell surface markers (see, e.g., Tables 2-4), or fragments thereof, for example, extracellular portions thereof in the case of transmembrane proteins, for example, by attaching the one or more cell surface markers, extracellular portions or ligand binding regions thereof to the particle via a free amine, free carboxyl and/or free hydroxyl group present on the surface of the particle. Functionalization of a particle with a dye or cell surface molecule can also occur through a linker, for example a streptavidin/biotin conjugate.

Depending on the target cell, individual particles can be derivatized with one or more biomolecules, including cell surface markers, or fragments thereof, for example, extracellular portions thereof in the case of transmembrane proteins to further mimic the structural properties of a target cell or to impart the synthetic particle with a desired biological function. Tables 2-4, provided below, set forth a non-limiting list of cell surface markers that can be used to derivative particles. Although the cell surface marker is provided, it is understood that a portion of the cell surface marker, for example, a receptor binding portion, a ligand binding portion, or an extracellular portion of the marker can be used to derivative the particle (at the free functional group, as described above). In some embodiments, the particles of the present disclosure mimic target cells as measured by experimental assays. In other embodiments, the particles mimic the properties of one or more target cells, as exhibited in a biological context. Thus, in some embodiments, the particles of the present disclosure exhibit immunostimulatory or feeder properties.

TABLE 2

| | | Cell Surface Marker(s) |
|---|---|---|
| Target Cell | Cell Surface Marker(s) (human) | (mouse) |
| B Cell | CD19, CD20 | CD19, CD22 (B cell activation |
| | | marker), CD45R/B220 |
| T Cell | CD3, CD4, CD8 | CD3, CD4, CD8 |
| Activated T Cells | CD25, CD69 | CD25, CD69 |
| Dendritic Cell | CD1c, CD83, CD123, CD141, | CD11c, CD123, MHC II |
| | CD209, MHC II | |
| Plasmacytoid | CD123, CD303, CD304 | CD11c^{int}, CD317 |
| Dendritic Cells* | | |
| Platelet (resting) | CD42b | CD41 |
| Platelet (activated) | CD62P | CD62P |
| Natural Killer | CD16, CD56 | CD49b (clone DX5) |
| Cells | | |
| Hematopoietic | CD34, CD90 | CD48, CD117, CD150, Sca-1 |
| Stem Cell | | |
| Macrophage | CD11b, CD68, CD163 | F4/80, CD68 |
| Monocyte | CD14, CD16, CD64 | CD11b, CD115, Ly-6C |
| Plasma Cell | CD138 | CD138 |
| Red Blood Cell | CD235a | TER-119 |
| Neutrophil | CD15, CD16 | CD11b, Ly-6B.2, Ly6G, Gr-1 |
| Basophil | 2D7 antigen, CD123, CD203c, | CD200R3, FcεRIα |
| | FcεRIα | |
| Eosinophil | CD11b, CD193, EMR1, Siglec-8 | CD11b, CD193, F4/80, Siglec-F |
| Granulocyte | CD66b | CD66b, Gr-1/Ly6G, Ly6C |
| Endothelial cell | CD146 | CD146 MECA-32, CD106, |
| | | CD31, CD62E (activated |
| | | endothelial cell) |
| Epithelial cell | CD326 | CD326 (EPCAM1) |
| Natural Killer | CD56 | CD335 (NKp46) |
| (NK) cell | | |
| Myeloid derived | CD11b, CD14, CD33 (Siglec-3) | CD11b, GR1 |
| suppressor cell | | |
| (MDSC) | | |
| APC/Immune cell | Anti CD3, anti CD28, and | Anti CD3, anti CD28, and |
| activation | optionally CD19 | optionally CD19 |

TABLE 3
B cell maturation markers for use
with the particles described herein

| B-cell type | Cell surface marker(s) |
|---|---|
| Pro-B | CD19, CD20, CD34, CD38, CD45R |
| Pre-B | CD19, CD20, CD38, CD45R |
| Immature B | CD19, CD20, CD40, CD45R, IgM |
| Tr-B | CD10, CD19, CD20, CD24, CD28 |
| Naïve-B | CD19, CD20, CD23, CD40, CD150 (SLAM), IgD, IgM |
| B-1 | CD19, CD20, CD27, IgM |
| Memory B | CD19, CD20, CD28, CD40, IgA, IgG |
| Plasma Cell | CD9, CD28, CD31, CD38, CD40, CD95 (FAS), |
| | CD184 (CXCR4) |

TABLE 4
Cell surface markers for use with the particles described herein

| | | |
|---|---|---|
| 14-3-3 [alpha][beta] | Cdc-123 | HPx2 |
| 14-3-3 [epsilon] | Cdc-2 (p34) | Hrk |
| 14-3-3 [zeta] | Cdc-25A Phosph (Ser17) | Hsc70 |
| 14-3-3 [theta] | Cdc-25C | HSD17B1 |
| 14-3-3 [sigma] | Cdc-37 | HSD3B1 |
| 15-Lipoxygenase 1 | Cdc-45L | HSF1 |
| 160 kD Neurofilament | Cdc-6 | HSF2 |
| Medium | | |
| 200 kD Neurofilament | CDc-7 | HSF4 |
| Heavy | | |
| 2H2 | Cdk1 | HSL |
| 3G11 sialoganglioside | Cdk2 | Hsp105 |
| antigen | | |
| 4E-BP1 | Cdk4 | Hsp14 |
| 4E-BP1 Phospho (Thr37/46) | Cdk5 | Hsp22 |
| 5-Methylcytidine | Cdk6 | HSP25 |
| 5HT3A receptor | Cdk7 | Hsp27 |
| 5T4 | Cdk9 | Hsp40 |
| 68 kDa Neurofilament | CdkA1 | Hsp47 |
| Light | | |
| 7.1 | CdkN2A | Hsp60 |
| 70 kD Neurofilament Light | CdkN3 | Hsp70 |
| A20 | CDT1 | Hsp70-2 |
| A2B5 | CDX2 | Hsp90 |
| AAK1 | CEACAM19 | Hsp90[alpha] |
| ABCA1 | CEACAM20 | Hsp90[beta] |
| ABCA7 | CEACAM7 | HspA4 |
| ABCB4 | CEBP[alpha] | HspA6 |
| ABCB5 | CEBP[beta] | HSPA9 |
| ABCC10 | CEND1 | HspB2 |
| ABCC11 | CENPA | HspB7 |
| ABCG1 | CENPE | HSV tag |
| ABI2 | CENPF | HTLV I gp46 |
| ABIN3 | CENPH | HTLV I p19 |
| ABIN3[beta] | Centrin 2 | HtrA2 / Omi |
| ABL2 | CFAH | Human Papillomavirus 16 (E7) |
| Abraxas | cFos | Huntingtin |
| ACAA1 | CFTR | HUS1 |
| ACADM | CGB5 | Hydrogen Potassium ATPase [beta] |
| ACAT2 | cGK1 | I-Ak (A[alpha]k) |
| ACBD3 | CH2 | I-Ak (A[beta]k) |
| ACD | CHCHD5 | Ia (B cells) |
| ACE2 | CHD3 | IBA1 |
| Acetyl Coenzyme A | CHD4 | IBP2 |
| Carboxylase | | |
| Acetyl Coenzyme A | Chemerin | ICAD |
| Carboxylase [alpha] | | |
| Acetyl Coenzyme A | CHIPS, C-terminus | IDO |
| Synthetase | | |
| Acetylated Lysine | CHIPS, N-terminus | IFABP |
| AChR[alpha] | Chk1 | IFN-[alpha] |
| AChR[beta] | Chk2 | IFN-[alpha]1 |
| AChR[gamma] | Chondroitin Sulfate | IFN-[alpha]2[beta] |
| Aconitase2 | CHOP | IFN-[beta] |
| ACOT12 | Chromogranin C | IFN-[gamma] |
| ACSA2 | ChT1 | IFN-[gamma]R[beta] |
| ACSF2 | chTOG | IFN-Î © |
| ACSM5 | cIAP1 | IFNA1 |
| Act1 | cIAP2 | IFNAR1 |
| Activation molecule 8 (B | CIAS1 | IFT88 |
| cells) | | |
| Activin A Receptor Type | CIDEA | Ig |
| IB | | |
| Activin A Receptor Type | CIP4 | Ig (polyspecific) |
| IIB | | |
| ACTN3 | CISD1 | Ig light chain Îº |
| ACY1 | CITED1 | Ig light chain Î» |
| ACY3 | CITED2 | Ig light chain Î»1, Î»2, Î»3 |
| ADA | cJun | IgA |
| ADAM12 | cJun Phospho (Tyr91/Tyr93) | IgA (Fab2) |
| ADE2 | CKII[alpha] | IgA (H) |
| Adenosine A1 Receptor | CKMT2 | IgA, Îº |
| Adenosine A2aR | CLASP1 | IgA, Î» |
| Adenovirus | Clathrin | IgA1 |
| Adenovirus Fiber | Claudin-1 | IgA2 |
| monomer and trimer | | |
| Adenovirus hexon protein | Claudin-10 | IgD |
| Adenylate Kinase 1 | Claudin-15 | IgD ([delta] heavy chain) |
| Adenylosuccinate Lyase | Claudin-16 | IgDa |
| ADFP | Claudin-18 (C-term) | IgDb |
| ADH1B | Claudin-18 (Mid) | IgE |
| ADH6 | Claudin-4 | IgE, Îº |
| ADH7 | Claudin-5 | IgEa |
| ADI1 | Claudin-8 | IgEb |
| Adiponectin | CLAW-H | IgG |
| Adiponectin Receptor 2 | CLEC12A | IgG (Fab H/L) |
| Adipose Triglyceride | CLEC1B | IgG (Fab) |
| Lipase | | |
| ADP Ribosylation Factor | CLEC4A | IgG (Fab2 Fc) |
| ADP-ribosyltransferase 2.2 | CLEC4M | IgG (Fab2 H/L) |
| gene | | |
| Adrenodoxin | CLEC9A | IgG (Fab2) |
| AF10 | CLIP | IgG (Fc) |
| AFAP1 | CLOCK | IgG (H/L) |
| AFP | Clostridium botulinum Toxin | IgG ([gamma] chain specific) |
| | B | |
| AG2 | CLPP | IgG Fd |
| AGAP1 | cMaf | IgG light chain |
| AGPAT5 | cMet | IgG, Îº |
| AGR2 | CMKLR1 | IgG/IgM |
| AHSG | CMRF44 | IgG/IgM/IgA |
| AICDA | CMRF56 | IgG/IgM/IgA (Fab2 H/L) |
| AID | cMyb | IgG/IgM/IgA (Fab2) |
| AIF | cMyc | IgG/IgM/IgA (H/L) |
| AIM-2 | CNDP2 | IgG/IgY |
| Aiolos | CNTFR[alpha] | IgG1 |
| AIPL1 | COASY | IgG1 (heavy chain) |
| AIRE | Coatomer [delta] | IgG1, Îº |
| AK3 | Cofilin | IgG1, Î» |
| AK3L1 | Colec12 | IgG1/2a |
| AK5 | Collagen I | IgG1/3 |
| Akt | Collagen I/III | IgG1a |
| Akt (pS473) | Collagen II | IgG1b |
| Akt (pT308) | Collagen III | IgG2 |
| Akt1 | Collagen IV | IgG2, Îº |
| Akt2 | Collagen V | IgG2, Î» |
| Akt3 | Collagen VI | IgG2/3 |
| Albumin | Collagen VII | IgG2a |
| Alcohol Dehydrogenase | COMMD1 | IgG2a, Îº |
| Aldehyde Reductase | Complement Factor B | IgG2a, Î» |
| ALDH1A1 | Complex I Immunocapture | IgG2a/b |
| ALDH1L1 | Conjugated Choline Glutaric | IgG2b |
| | acid | |
| ALDH2 | Connexin 26 | IgG2b, Îº |
| ALDH3A1 | Connexin 30 | IgG2c |
| ALDH3A2 | Connexin 30.2 | IgG2c, Îº |
| ALDH5A1 | Connexin 30.3 | IgG3 |
| ALDH6A1 | Connexin 32 | IgG3, Îº |
| ALDH7A1 | Connexin 36 | IgG3, Î» |
| ALDOB | Connexin 37 | IgG4 |
| Aldolase B | Connexin 37 (C-term) | IgGDa |
| Alexa Fluor ® 405/Cascade | Connexin 37 (Mid) | IgK |
| Blue | | |
| Alexa Fluor ® 488 | Connexin 39 | IGKC |
| ALG2 | Connexin 39 (Mid) | IL |
| Alix | Connexin 40 (C-term) | IGLC2 |
| Allergin1 | Connexin 40 (Mid) | IgM |
| alpha 1 Antitrypsin | Connexin 43 | IgM (Fab2) |
| alpha 1 Catenin | Connexin 45 | IgM (Fc) |
| alpha 1 Sodium Potassium | Connexin 45 (C-term) | IgM (H/L) |
| ATPase | | |
| alpha 2 Catenin | Connexin 46 | IgM, Îº |
| alpha 2 Macroglobulin | Connexin 47 | IgM, Î» |
| alpha Actin 1 | Connexin 57 (C-term) | IgMa |
| alpha Actin 2 | Connexin 57 (Mid) | IgMb |
| alpha Actinin | Contactin 2 | IgY |
| alpha Actinin 2 | COPS3 | Igâ€ ™s |
| alpha Actinin 3 | Coronavirus | Ihh |
| alpha Actinin 4 | Coronin 1A | Ikaros |
| alpha Adaptin | Coronin 1B | IkB[alpha] |
| alpha Adducin | Cortactin | IkB[beta] |
| alpha B Crystallin | Cortical Thymocytes | IkB[zeta] |
| alpha Fodrin | COX I | IKK[alpha] |
| alpha Internexin | COX I/III | IKK[beta] |
| alpha Synuclein | COX II | IKK[gamma] p(S376) |
| ALS1 | COX IV | IKK[epsilon] |
| AMACR | COX VA | IL-10 |
| Aminopeptidase P | COX VIA1 | IL-11R[alpha] |
| AML1 | Coxsackie Adenovirus | IL-12 |
| | Receptor | |
| Amphiphysin | CPF | IL-12 (p35) |
| AMPK[alpha] | CPI17[alpha] | IL-12 (p70) |
| AMPK[alpha]1 | Cpn10 | IL-12 R[beta]1 |
| AMPK[alpha]2 | CPO | IL-12 R[beta]2 |
| AMPK[beta]1 | CPS1 | IL-12/IL-23 (p40) |
| AMPK[gamma]1 | CPT2 | IL-13 |
| Amyloid[beta] 42 | CRABP1 | IL-15 |
| ANAPC2 | CRABP2 | IL-15/IL-15R |
| AND1 | CRALBP | IL-15R[alpha] |
| Androgen Receptor | Creatine Kinase BB | IL-16 |
| Angiotensin I | Creatine Kinase MM | IL-17D |
| Angiotensin II Receptor 2 | CREB | IL-17A |
| Angiotensin III | CREB Phospho (Ser133) | IL-17A/F |
| ANKRD53 | cRel | IL-17B |
| Annexin IV | Cripto1 | IL-17C |
| Annexin V | CRISP3 | IL-17E |
| ANP | Crk p38 | IL-17F |
| Anti-Kudoa thrysites | CrkL | IL-18 |
| Anti-T. brucei procyclin | CrkL (pY207) | IL-18BP |
| (GPEET) | | |
| Anti-T. brucei procyclin | CROT | IL-19 |
| (phosphorylated GPEET) | | |
| Antiglobulin (Coombs) | CRRY | IL-1RA |
| Antithrombin III | CRTAM | IL-1RN |
| AP2 [alpha] | CRTC3 | IL-1[alpha] |
| AP2 [alpha][beta] | CRY2 | IL-1[beta] |
| AP2 [gamma] | Cryptochrome I | IL-2 |
| AP2M1 | Cryptosporidium | IL-20R2 |
| AP2S1 | Cryptosporidium Parvum | IL-20R[alpha] |
| APAF1 | CRYZL1 | IL-20R[beta] |
| APBB3 | CSK | IL-21 |
| APC | CSK Binding Protein | IL-22 |
| APC-1 | CSPS | IL-22R[alpha]2 |
| APC-10 | cSrc | IL-23 (p19) |
| APC-11 | CST2 | IL-23R |
| APC-2 | CTDSP1 | IL-24 |
| APC-3 | CTNNA3 | IL-25 |
| APC-5 | CTNNBL1 | IL-27 |
| APC-7 | Cullin 1 | IL-27 (p28) |
| APC-8 | Cullin 2 | IL-27R[alpha] |
| APE1 | Cullin 3 | IL-28 |
| APG12 | Cullin 4A | IL-28R[alpha] |
| APG3 | Cullin 4A/B | IL-29 |
| APG5 | Cullin 4B | IL-3 |
| APG7 | Cutaneous Lymphocyte | IL-31 |
| | Antigen | |
| APMAP | CUTL1 | IL-32[alpha][beta][gamma][delta] |
| Apo-2.7 | CX3CL1 | IL-32[alpha][beta][delta] |
| Apo-2.7 (7A6) | CX3CR1 | IL-33 |
| ApoE | CXCL1 | IL-34 |
| ApoE4 | CXCL10 | IL-4 |
| APOER2 | CXCL12[alpha] | IL-4R[alpha] |
| Apolipoprotein AI | CXCL12[beta] | IL-5 |
| Apolipoprotein AII | CXCL13 | IL-6 |
| Apolipoprotein AIV | CXCL9 | IL-7 |
| Apolipoprotein B | CXCR7 | IL-7R[alpha] |
| Apolipoprotein CIII | CXorf26 | IL-8 |
| Apolipoprotein D | Cyanine | IL-9 |
| Apolipoprotein E | CYB5R2 | ILF3 |
| Apolipoprotein F | CYB5R3 | ILK |
| Apolipoprotein H | Cyclin A | ILK1 |
| Apolipoprotein J | Cyclin A2 | ImmunofluorescenceN-[gamma] |
| Apolipoprotein L1 | Cyclin B1 | IMP3 |
| Apolipoprotein M | Cyclin B2 | Importin9 |
| Apoptotic neutrophils | Cyclin D1 | Influenza A Virus M2 Protein |
| APP | Cyclin D2 | Influenza B Virus Nucleoprotein |
| Aquaporin 1 | Cyclin D3 | ING1 |
| Aquaporin 5 | Cyclin E | ING2 |
| ARF1 | Cyclin E2 | ING3 |
| ARF5 | Cyclin H | ING4 |
| ARFGAP1 | Cyclins D1/D2/D3 | Inhibin [alpha] |
| ARFRP1 | Cyclophilin 40 | iNOS |
| Argonaute-1 | CYLD | INPP4A |
| ARH | CysLT1 | INPP4B |
| ARHGAP25 | Cystatin C | Insulin |
| ARHGAP4 | Cystatin S | Insulin Degrading Enzyme (IDE) |
| ARL11 | Cytochrome B245 heavy | Insulin Receptor R |
| | chain | |
| ARL5B | Cytochrome B245 light chain | Integrin [alpha]4/[beta]7 |
| ARPC5 | Cytochrome c | Integrin [alpha]9/[beta]1 |
| Artemis | Cytochrome P450 17A1 | Integrin [alpha]V/[beta]5 |
| Aryl hydrocarbon Receptor | Cytochrome P450 19A1 | Integrin [alpha]V/[beta]6 |
| ASB-1 | Cytochrome P450 1A2 | Integrin [beta]1 Phospho (Tyr783) |
| ASCC1 | Cytochrome P450 2A6 | Integrin [beta]1 Phospho (Tyr795) |
| ASCC2 | Cytochrome P450 2B6 | Integrin [beta]5 |
| ASGPR | Cytochrome P450 2C9 | Integrin [beta]6 |
| Asialo-GM1 | Cytochrome P450 2J2 | Integrin [beta]7 |
| ASK1 | Cytochrome P450 3A4 | Intercalated DNA |
| Asparagine synthetase | Cytochrome P450 3A5 | Intra Acrosomal Protein |
| Ataxin 1 | Cytochrome P450 Reductase | Intra-Acrosomal Proteins |
| ATF1 | Cytokeratin | Invariant NK T |
| ATF2 | Cytokeratin (acidic) | IP10 |
| ATG4A | Cytokeratin (basic) | IQGA1 |
| ATG9A | Cytokeratin (Pan-reactive) | IRAK1 |
| ATIC | Cytokeratin 1 | IRAK3 |
| Atlantic Salmon Ig | Cytokeratin 10 | IRAK4 |
| ATM | Cytokeratin 10/13 | IRE1 |
| ATP citrate lyase | Cytokeratin 13 | IRF1 |
| ATP1B3 | Cytokeratin 14 | IRF3 |
| ATP5A | Cytokeratin 14/15/16/19 | IRF4 |
| ATP5H | Cytokeratin 15 | IRF5 |
| ATP5J | Cytokeratin 16 | IRF6 |
| ATP5O | Cytokeratin 17 | IRF7 |
| ATP6V0D1 | Cytokeratin 18 | IRF7 (pS477/pS479) |
| ATP6V1B1 | Cytokeratin 19 | IRF8 |
| ATPB | Cytokeratin 2 | IRF9 |
| ATRIP | Cytokeratin 20 | IRS1 |
| Aurora A | Cytokeratin 4 | IRS1 (pY896) |
| Aurora A Phospho | Cytokeratin 4/5/6/8/10/13/18 | IRS2 |
| (Thr288) | | |
| Aurora B | Cytokeratin 40 | IRS4 |
| Aurora B Phospho | Cytokeratin 5 | ISG15 |
| (Thr232) | | |
| AVEN | Cytokeratin 5/6/18 | ISG20 |
| Avian Influenza A | Cytokeratin 5/8 | ISL1 |
| Neuraminidase | | |
| Avidin | Cytokeratin 6 | Isthmin1 |
| Axin 2 | Cytokeratin 6a | ITCH |
| Axl | Cytokeratin 7 | Integrin [alpha]7 |
| B and Activated T Cells | Cytokeratin 7/17 | ITK |
| B Cell | Cytokeratin 8 | ITPR1 |
| B Cell Subset | Cytokeratin 8/18/19 | Jagged2 |
| B cells (pan reactive) | D4-GDI | JAK2 |
| B lymphocytes antibody | DAB2 | JAK3 |
| [UCH-B1] | | |
| b-Endorphin | DACH1 | JAM2 |
| B-Raf Phospho | DAND5 | JAML |
| (Thr598/Ser601) | | |
| B18R | DAP1 | Japanese encephalitis virus NS1 |
| | | glycoprotein |
| B7-H4 | DAP12 | JNK |
| BACE1 | DAPK1 | JNK Phospho (Thr183/Tyr185) |
| BACE2 | DAPK2 | JNK1/JNK2/JNK3 |
| BACH1 | DARPP32 | JNK2 |
| baculovirus envelope gp64 | Daxx | Junctional Adhesion Molecule C |
| protein | | |
| BAG1 | DAZL | Junctophilin-1 (C-term) |
| BAG2 | DBC1 | Junctophilin-1 (Mid) |
| BAG3 | DCAMKL1 | Junctophilin-2 (C-term) |
| BAG4 | DCC | Junctophilin-3 (C-term) |
| BAIAP2 | DCIR2 | KAP1 |
| BAK | DCLRE1B | KATNA1 |
| BAMBI | DCP1a | KCNH1 |
| BAP31 | DcR3 | KDEL |
| BAP37 | DCTN2 | KDM4D |
| basal cell Cytokeratin | DcTRAIL-R1 | Ki-67 |
| Basophils | DcTRAIL-R2 | KIF22 |
| Bassoon | DCXR | KIF3A |
| BATF | DDB1 | KIF4A |
| Bax | DDDDK tag | KIFA3 |
| BCAR1 | DDX3 | Kindlin2 |
| BCAR2 | DDX4 | Kinetoplastid Membrane Protein 11 |
| | | (KMP-1)) |
| BCKD complex E2 subunit | DDX50 | KIR-2.1 |
| Bcl-10 | DECR1 | KIR-2D (pan CD158) |
| Bcl-2 | Dectin1 | KLF4 |
| Bcl-2 (pS70) | Dectin2 | KLF6 |
| Bcl-2 like 12 | DEF8 | KLH |
| Bcl-2 like 2 | Defensin [alpha]1 | KLHL11 |
| Bcl-22 | DELETE | KLRA3 |
| Bcl-2A1 | delta 1 Catenin | KLRC1 |
| Bcl-2[alpha] | Delta like protein 1 | KLRG1 |
| Bcl-3 | Delta like protein 4 | KMT4 |
| Bcl-6 | Delta Opioid Receptor | KMT5A |
| Bcl-xL | DeltaC | KOR-SA3544 |
| Bcl-XS/L | DeltaD | KS1/4 |
| BCR | Dendritic Cell Marker | Ksp37 |
| BCSC1 | Deoxycytidine kinase | KSR1 |
| BDH2 | Desmin | Ku70 |
| BDKRB2 | Desmoglein 2 | Ku70/80 |
| BDNF | Desmoglein1 | Ku80 |
| Beclin1 | Desmoplakin | Kudoa Thyrsites |
| Bestrophin 3 | Destrin | Kunitz Protease Inhibitor |
| beta 2 Adrenoreceptor | Dextran | Kv4.2 |
| Beta 3 Adrenergic | DGKA | L/S-MAG |
| Receptor | | |
| beta 3 Sodium Potassium | Dicer | Labeling Check Reagent |
| ATPase | | |
| beta Actin | DISC1 (C-term) | Lactate Dehydrogenase |
| beta Arrestin 1 | DISC1 (Mid) | Lactate Dehydrogenase B |
| beta Arrestin 2 | Dishevelled 3 | Lambda |
| beta Catenin | Disialoganglioside GD2 | Lamin A |
| beta Catenin (npaa 27-37) | Disialoganglioside GD3 | Lamin A/C |
| beta Catenin (npaa 35-50) | Dkk1 | Lamin B Receptor |
| beta Catenin (pS45) | Dkk3 | Lamin B1 |
| beta Dystroglycan | DLC8 | Lamin B2 |
| beta galactosidase | DLK1 | Lamin C |
| beta galactosidase fusion | Dlx5 | Laminin |
| proteins | | |
| beta Synuclein | DM-GRASP | Laminin 5 |
| beta2 Microglobulin | DMT1 | Laminin Receptor |
| BHMT | DNA-PKcs | Laminin [beta]1 |
| Bid | DNA-PKcs Phospho | LAMP2a |
| | (Thr2609) | |
| Biglycan | DNAI1 | LAMP2b |
| Bilirubin Oxidase | DNAJA2 | LAT |
| Bim | DNAJB2 | LAT (pY171) |
| BimL | DNAJC3 | LAT (pY226) |
| BIN1 | DNAPK | LBP |
| BIN3 | DNM1L | LC3 |
| Biotin | Dnmt1 | LC3B |
| BiP | Dnmt3b | LCAT |
| BLBP | DNP | Lck |
| Blimp-1 | DOK2 | Lck (pY505) |
| BLK | DOK7 | LDH1 |
| BLNK | Dopamine Receptor D1 | LDH1/B/C |
| BLNK (pY84) | Dopamine Receptor D3 | LDL (MDA oxidized) |
| Blood Group A Antigen | Dopamine Receptor D5 | LDLR |
| Blood Group AB Antigen | Dopamine [beta] Hydroxylase | LEF1 |
| Blood Group B Antigen | Doublecortin | Leishmania LPG (repeat epitope) |
| Blood Group H ab Antigen | DP1 | Leishmania Major Surface Protease |
| | | (GP-63) |
| Blood Group H ab | DPH2 | LEKTI |
| Antigen/n Antigen | | |
| Blood Group H inhibitor | DPP10 | Leukemia Inhibitory Factor |
| Blood Group Lewis a | DPP3 | Leukotriene A4 hydrolase |
| Blood Group M Antigen | DPP9 | Leukotriene B4 Receptor |
| Blood Group N Antigen | Dppa4 | LHX3 |
| Blooms Syndrome Protein | DPYD | LI-Cadherin |
| Blm | | |
| BM1 | DR3 | LIF |
| BMAL1 | DRAK1 | DNA Ligase I |
| BMI1 | DRAK2 | DNA Ligase III |
| Bmk | Drebrin | LIM kinase 2 |
| BMP15 | DTYMK | LIME1 |
| BMP4 | DUSP23 | LIMK1 |
| BMP7 | DUSP27 | LIMS1 |
| BMPR1A | DUSP3 | Lin28 |
| BMPR2 | DUSP5 | Lineage Cocktail |
| BMX | DUSP6 | Lipin 1 |
| bMyc | DUX4 | LIS1 |
| BNIP2 | DYKDDDDK Epitope Tag | Liver Carboxylesterase 1 |
| BNIP3 | Dynamin | LKB1 |
| BNIP3L | Dynamin1 | LMO2 |
| BOB1 | Dynamitin | LOX |
| BORA | Dynein light chain 2 | LOX1 |
| Borealin | Dysbindin | LRP5/6 |
| Borrelia burgdorferi | Dysferlin | LRP6 |
| BPI | Dystrobrevin [alpha] | LRPAP1 |
| BRaf | Dystrobrevin [beta] | LSD1 |
| BRCA1 | Dystroglycan Phospho | LSP1 |
| | (Tyr893) | |
| BRCC36 | E. Coli O/E | LSS |
| BRD3 | E2A-Pbx1 | LT[alpha] |
| BrdU | E2F1 | Luciferase |
| BRF1 | E47 | LXR[alpha] |
| BRG1 | E4BP4 | Ly-108 |
| BRN3A | Ea52-68 peptide bound to I-A | Ly-49A |
| Btk | Ea52-68 peptide bound to the | Ly-49A/D |
| | I-A | |
| Btk (pY551)/Itk (pY511) | EAAT1 | Ly-49AB6 |
| BTLN-2 | Early B Lineage | Ly-49C/F/I/H |
| BTN1A1 | EBF1 | Ly-49C/I |
| Bu1 | EBI3 | Ly-49D |
| Bu1a | EBP50 | Ly-49E/F |
| Bu1a/Bu1b | ECGF1 | Ly-49F |
| Bu1b | ECH1 | Ly-49G |
| BubR1 | ECRG4 | Ly-49G2 |
| Bulb | EDA | Ly-49G2B6 |
| Butyrylcholinesterase | EDA-A2R | Ly-49H |
| C peptide | EDG1 | Ly-49I |
| C reactive protein | EDG2 | Ly-51 |
| C/EBP[beta] | EDG3 | Ly-6A.2/Ly-6E.1 |
| C1 Inhibitor | EDG6 | Ly-6A/E |
| C15orf40 | EEA1 | Ly-6b |
| C16orf72 | EEF1G | Ly-6B.2 |
| C1orf50 | EEF2 | Ly-6C |
| C1Q | EEF2K | Ly-6D |
| C1QA | EEN | Ly-6G |
| C1QB | EFEMP1 | Ly-6G/C |
| C1QC | EFEMP2 | Ly-6K |
| C1QG | Eg5 | Ly-77 |
| C1r | Eg5 Phospho (Thr927) | Lymphotoxin [beta] |
| C1s | EGF | Lymphotoxin [beta] Receptor |
| C20orf30 | EGF Receptor | Lyn |
| C20orf43 | EGF Receptor (pY1173) | LYRIC |
| C21orf56 | EGF Receptor (pY845) | Lysophospholipase 1 |
| C21orf59 | EGF Receptor (pY992) | Lysosomal acid lipase |
| C2orf43 | EGR1 | Lysozome |
| C3 | EGR2 | Lysozyme |
| C3aR | EHD1 | Lyve1 |
| C3b | eIF1 | M-CSF |
| C3c | eIF2C2 | M13 Bacteriophage Coat Protein |
| | | g8p |
| C3d | EIF2S1 | M13 Bacteriophage Protein |
| C4 | eIF2[gamma] | MAA |
| C4 binding protein | eIF3 | Mac-2BP |
| C4b | eIF3D | macroH2A.1 |
| C4c | eIF3D (p66) | Macrophage |
| C4d | eIF3F | Macrophage Activator |
| C4orf42 | eIF3G | Macrophage galactose lectin |
| C5 | eIF3H (p40) | Macrophage/Granulocyte |
| C5aR1 | eIF3I (p36) | Macrophages/Monocytes |
| C5L2 | eIF3J | MAD2 |
| C6 | eIF3K | MadCAM1 |
| C6orf64 | eIF4B | MADD |
| C8A/B/G | eiF4E | MADH7 |
| C9 | eIF4E (pS209) | MAFB |
| C9orf41 | eIF4E2 | MAG |
| CA125 | eIF5A | MAGE-A |
| CA19.9 | eIF6 | MAGE1 |
| CAB39 | Elastase | MAIR2 |
| CACNA1S | Elk1 | MAIR4 |
| CACNA2 | Elk1 (pS383) | MALT1 |
| CACNG1 | ELK3 | Mammaglobin A |
| CAD | Elongin B | MAP1LC3A |
| Cadherin 1 | Elongin C | MAP2 |
| Cadherin 10 | EMAP II | MAP2B |
| Cadherin 11 | Embigin | MAP2K1IP1 |
| Cadherin 7 | EMG1 | MAP3K8 |
| Cadherin 8 | Emi1 | MAP4 Phospho (Ser768) |
| Cadherin 9 | EMR3 | MAP4K1 |
| Cadherin E | EMSY | MAP4K4 |
| Cadherin H | Ena/Vasp-like | MAPK12 |
| Cadherin K | EndoG | MAPK6 |
| Cadherin P | EndoGlyx-1 | MAPKAP Kinase 2 |
| Cadherin R | Endomucin | MAPKAP Kinase 2 Phospho |
| | | (Thr334) |
| CAK C Terminus | Endothelial Cells | MARCKS |
| CAK N Terminus | Endothelial Lipase | MARCO |
| CAK Phospho | Endothelial Venule Marker | Marginal Zone B Cells |
| (Ser164/Thr170) | | |
| Calbindin | Endothelium | MARK2 |
| Calcineurin A | Engrailed1 | MARK3 |
| Calcitonin Receptor | ENO1 | MART1 |
| Calcium Sensing Receptor | Enolase1 | Mast Cell |
| Caldesmon | eNOS | Mast Cell Protease 11 |
| Calgranulin A | eNOS (pS1177) | mature macrophage marker |
| Calgranulin B | Entpd2 | MBD1 |
| Calmodulin | Eomes | MBD2 |
| Calnexin - ER membrane | Eos | MBL |
| marker | | |
| Calpain 1 | Epac1 | MCL1 |
| Calpain 2 | Eph Receptor A1 | MCM2 |
| Calpain 9 | Eph Receptor A2 | MCM3 |
| Calpain S1 (small subunit) | Eph Receptor A4 | MCM4 |
| Calpastatin | Eph Receptor B4 | MCM5 |
| Calponin | Eph Receptor B6 | MCM6 |
| Calreticulin | Ephrin A2 | MCM7 |
| Calretinin | Ephrin A3 | MCP-1 |
| Calsequestrin 2 | EPHX2 | MCP-4 |
| CaMKI | EPM2AIP1 | MCP-8 |
| CaMKII | EPOR | MCSF |
| CaMKII Phospho (Thr286) | EPS15R | MD1 |
| CaMKII[delta] | Epsin 1 | MD2 |
| CamKIV | Epsin 2 | MDC |
| CaMKI[alpha] | ER-HR3 | MECT1 |
| CAMLG | ER-MP54 | MEF2A |
| CAMP Protein Kinase | ER-TR7 | MEIS1 |
| Catalytic subunit | | |
| CAMP Protein Kinase | ER81 | MEK1 |
| Catalytic subunit [alpha] | | |
| Cannabinoid Receptor I | ERAB | MEK1 (p298) |
| Cannabinoid Receptor II | ERCC1 | MEK1 (pS218)/MEK2 (pS222) |
| CAP-G2 | ERG | MEK1/2 (pS222) |
| CAP18 | ERK1 | MEK2 |
| CAP2 | ERK1/2 (pT185/pY187) | MEK3 |
| CAP3 | ERK1/2 (pT202/pY204) | MEK4 |
| Carbonic Anhydrase I | ERK1/ERK2 | MEK5 |
| Carbonic Anhydrase IX | ERK2 | MEK6 |
| Carboxylesterase 1 | ERK5 | MEK7 |
| Carboxypeptidase A1 | ERMAP | MEKK1 |
| Carboxypeptidase A2 | ERp29 | MEKK2 |
| CARD11 | ERp72 | MEKK3 |
| CARD8 | Erythroid Cells | MEKK4 |
| CARD9 | Erzin/Radixin/Moesin | Melanoma |
| Cardiac Troponin T | ER[alpha] Phospho (Ser167) | MELK |
| CARKL | ESAM | MEMO1 |
| CARM1 | Estrogen Inducible Protein | Mena |
| | pS2 | |
| Casein Kinase 1 [alpha] | Estrogen Receptor | Menin |
| Casein Kinase 1 [gamma]2 | Estrogen Receptor [alpha] | MEOX2 |
| Casein Kinase 2 [beta] | Estrogen Receptor [beta] | Merlin |
| Caspase 1 | Estrogen Related Receptor | MERTK |
| | alpha | |
| Caspase 10 | ETAR | Mesothelin |
| Caspase 11 | Ethenoadenosine | Metallothionein |
| Caspase 12 | ETS1 | MetRS |
| Caspase 2 | EVI2A | mGluR5 |
| Caspase 2L | EVI2B | MGMT |
| Caspase 3 | EWSR1 | MHC Class I |
| Caspase 4 | EXD1 | MHC Class I (H-2Db) |
| Caspase 5 | EXOSC3 | MHC Class I (H-2Dd) |
| Caspase 6 | EXOSC7 | MHC Class I (H-2Dk) |
| Caspase 7 | EYA2 | MHC Class I (H-2Dq/Lq) |
| Caspase 8 | EZH1/2 | MHC Class I (H-2Kb) |
| Caspase 9 | Ezrin | MHC Class I (H-2Kb/Db) |
| Catalase | Ezrin (pY353) | MHC Class I (H-2Kb/Dd) |
| Catechol-O- | F-actin | MHC Class I (H-2Kd a3 domain) |
| methyltransferase | | |
| Cathepsin D | F10A1 | MHC Class I (H-2Kd) |
| Cathepsin K | F4/80 | MHC Class I (H-2Kd/Dd) |
| Cathepsin L | FAA4 | MHC Class I (H-2Kd/Dd/q/u/v) |
| Caveolin1 | FABP4 | MHC Class I (H-2Kk) |
| Caveolin1 (pY14) | Factor I | MHC Class I (H-2Kq) |
| Caveolin2 | Factor IX | MHC Class I (H-2Ks) |
| Cbl | Factor VIII.vWF (delete) | MHC Class I (H-2Ld) |
| CBP | Factor XIIIa | MHC Class I (H-2Ld/Db) |
| CBWD1 | FADD | MHC Class Ib (H2-M3) |
| CBX1 | FAHD2A | MHC Class II |
| cCbl (pY700) | FAK | MHC Class II (DQ) |
| cCbl (pY774) | FAK (pS910) | MHC Class II (DR) |
| CCDC98 | FAM119A | MHC Class II (I-A) |
| CCK4 | FAM175A | MHC Class II (I-A/E) |
| CCL11 | FAM84B | MHC Class II (I-Ab) |
| CCL17 | FAM91A1 | MHC Class II (I-Ab/Ad) |
| CCL18 | FANCC | MHC Class II (I-Ab/As) |
| CCL19-Fc | FANCD2 | MHC Class II (I-Ad) |
| CCL20 | Fanconi anemia D2 Phospho | MHC Class II (I-Ak) |
| | (Ser222) | |
| CCL21 | FAP | MHC Class II (I-Ak/Ad/Ab/Aq/Ar) |
| CCL25 | Fascin | MHC Class II (I-Ak/As) |
| CCL3 | FBP1 | MHC Class II (I-Ap) |
| CCL5 | FBXO21 | MHC Class II (I-Aq) |
| CCL6 | FBXO31 | MHC Class II (I-E) |
| CCNB1IP1 | FBXO42 | MHC Class II (I-EÎº) |
| CCR10 | FBXO43 | MHC Class II (RT1B) |
| CCR11 | Fc Receptor Binding Inhibitor | MHC Class II (RT1Bu) |
| CCRD6 | Fc receptor IgA + IgM | MHC Class II (RT1D) |
| CCRL2 | FcR | MHC Class II [beta] |
| CD1 | FcRL6 | MHC Qa1b |
| CD1.1 | FcRLA | MICA |
| CD10 | Fc[epsilon]RI | MICA/MICB |
| CD100 | FDC | MICB |
| CD101 | FDFT1 | Microfold (M) Cells |
| CD102 | FDPS | Microtubule Associated Protein 2ab |
| CD103 | FE65 | Microtubule Associated Protein |
| | | RP/EB 2 |
| CD104 | FeLV p27 | Midkine |
| CD105 | FEN1 | Mineralocorticoid Receptor |
| CD106 | FER | MIP-1[beta] |
| CD107a | Ferritin Heavy Chain | MIPEP |
| CD107b | Ferritin Light Chain | Mitochondria |
| CD108 | Ferritin, mitochondrial | Mitofilin |
| CD109 | FES | Mitofusin 1 |
| CD11 | Fetal Hemoglobin | Mitofusin 2 |
| CD110 | FGF acidic | Mitotic Cells |
| CD111 | FGF basic | MKK6 |
| CD112 | FGF21 | MLH1 |
| CD113 | FGFR1 | MLK3 |
| CD114 | FGFR2 | MLL1 |
| CD115 | FGR | MLLT11 |
| CD116 | FH | MMP1 |
| CD117 | FHL1 | MMP10 |
| CD118 | Fibrillarin | MMP11 |
| CD119 | Fibrillin | MMP12 |
| CD11a | Fibrinogen | MMP13 |
| CD11a, strain | Fibrinogen [alpha] chain | MMP14 |
| polymorphism | | |
| CD11a/CD18 | Fibrinogen [gamma] chain | MMP15 |
| CD11b | Fibrinopeptide A | MMP17 |
| CD11b/c | Fibrinopeptide B | MMP19 |
| CD11c | Fibroblast activation protein | MMP2 |
| | [alpha] | |
| CD11d | Fibroblast Surface Protein | MMP20 |
| CD120a | Fibroblasts/Epithelial cells | MMP21 |
| CD120b | Fibronectin | MMP26 |
| CD121a | Fibronectin Receptor | MMP3 |
| CD121b | Fibulin5 | MMP8 |
| CD122 | Ficolin B | MMP9 |
| CD123 | Filaggrin | Mnk1 |
| CD124 | Filamin A | mNOS |
| CD125 | FITC | MnSOD |
| CD126 | FITC/Oregon Green | Moesin |
| CD127 | FIV | Monoamine Oxidase B |
| CD129 | FIV gp120 | Monocyte/Granulocyte |
| CD13 | FIV gp95 | Mononuclear Phagocyte |
| CD130 | FIV p24 | Mouse Embryonic Fibroblast (mEF) |
| | | Feeder Cells |
| CD131 | FIV p24 gag | Mouse Lineage |
| CD132 | FKBP12 | MPP1 |
| CD133 | FKBP4 | MRCL3 |
| CD133/2 | FKBP6 | MRE11 |
| CD134 | FKBPL | MRGPR-X2 |
| CD135 | FLiC | MRI1 |
| CD136 | Flightless1 | MRP14 |
| CD137 | FLIP | MRP2 |
| CD137L | Flt3L | MRP3 |
| CD138 | Fluorescent Protein | MRP4 |
| CD139 | FLV gp70 | MRP5 |
| CD14 | FLYWCH2 | MRP6 |
| CD140a | FMC7 | MRP8 |
| CD140b | fMLP Receptor | MRP8/14 |
| CD140b (pY1009) | FMRP | MSC (W8B2) |
| CD140b (pY1021) | FNTA | MSC (W3D5) |
| CD140b (pY771) | FNTB | MSC (W5C5) |
| CD140b (pY857) | Follicular Dendritic Cells | MSC (W7C6) |
| CD141 | Fos | MSC/NPC |
| CD142 | FOXA1 | MSH2 |
| CD143 | FOXA2 | MSH6 |
| CD144 | FOXC2 | MSI2H |
| CD146 | FOXD3 | MSK1 |
| CD147 | FOXI1 | MST1 |
| CD148 | FOXJ1 | MST1/MST2 |
| CD15 | FOXM1 | MST3 |
| CD150 | FOXO1 | MST4 |
| CD151 | FOXO3A | MST4/MST3/STK25 |
| CD152 | FOXP1 | mTOR |
| CD153 | FOXP3 | Muc-16 |
| CD154 | FPRL1 | Muc-2 |
| CD155 | FR4 | Muc-3 |
| CD156c | Fra2 | Muc-4 |
| CD157 | Fragilis | Muc-7 |
| CD158a | FRAT1 | MULT-1 |
| CD158a/h | Frataxin | Munc13-4 |
| CD158b | Frequenin | Munc18 |
| CD158b1/b2/j | Frizzled-1 | MUPP1 |
| CD158d | FSH[alpha] | Mus81 |
| CD158e | FSH[beta] | Musashi1 |
| CD158e/k | FUK | Muscarinic Acetylcholine |
| | | Receptor 2 |
| CD158e1 | FUS | muscle Actin |
| CD158e1/e2 | FXYD3 | Muscleblind-like 1 |
| CD158f | FYB | MVP |
| CD158g | Fyn | MYBBP1A |
| CD158h | Fyn (pY528)/c-Src (pY530) | MYBPC3 |
| CD158i | Fyn-Related Kinase | Myc tag |
| CD158j | FZR1 | MyD88 |
| CD159a | G-CSF | Myelin Basic Protein |
| CD159c | G3BP | Myelin oligodendrocyte glycoprotein |
| CD15s | G6PD | Myelin PLP |
| CD16 | GAB1 | Myeloid Antigen |
| CD16/32 | GAB2 | Myeloid Cell Nuclear |
| | | Differentiation Antigen |
| CD16/56 | GABA B Receptor 2 | Myeloid Lineage |
| CD160 | GABARAP | Myocilin |
| CD161 | GAD65 | Myogenin |
| CD161a | GAD67 | Myosin heavy chain |
| CD162 | GADD34 | Myosin IIA |
| CD162R | Galacto-cerebroside | Myosin light chain 2 |
| CD163 | Galactocerebroside | Myosin light chain 3 |
| CD164 | Galectin 1 | Myosin light chain kinase |
| CD165 | Galectin 10 | Myosin Phosphatase |
| CD166 | Galectin 3 | Myosin Phosphatase 1/2 |
| CD167a | Galectin 4 | MYST2 |
| CD168 | Galectin 7 | NADH2 |
| CD169 | Galectin 8 | Naf1 |
| CD16b | Galectin 9 | NAK |
| CD17 | gamma Synuclein | Nanog |
| CD170 | Ganglioside GD2 | NAPE-PLD |
| CD171 | Ganglioside GD3 | NAT1 |
| CD172 | Ganglioside GM1 | Native Lipoteichoic Acid |
| CD172a | Gankyrin | Natriuretic Peptide Receptor A |
| CD172a/b | GAP | Natural Killer Cell |
| CD172b | GAP43 | Natural Killer Cell Activation |
| | | Structures |
| CD172g | GAPDH | NBS1 |
| CD173 | GARP | NC1.1 |
| CD177 | GAS2 | NCF4 |
| CD178 | GAS7 | Nck |
| CD178.1 | GAT2 | NCOA1 |
| CD179a | GATA1 | NCOA2 |
| CD179b | GATA2 | NCX1 |
| CD18 | GATA3 | NDUFAF1 |
| CD180 | GATA4 | NDUFB4 |
| CD181 | GATM | NDUFS3 |
| CD182 | GBA3 | NEDD8 |
| CD183 | GBE1 | NEK2 |
| CD184 | GBP1 | NEK6 |
| CD185 | GBP2 | NEK7 |
| CD186 | GBP5 | NEK9 |
| CD19 | GC1qR | NEK9 Phospho (Thr210) |
| CD191 | GCDFP15 | Nestin |
| CD192 | GCDH | NETO2 |
| CD193 | GCK1 | Neurabin1 |
| CD194 | GCLM | Neuregulin1 |
| CD195 | GCN2 | Neuregulin3 |
| CD195 (cytoplasmic) | GCN5 | Neuroblastoma |
| CD195 Phospho (Ser337) | GCTM2 | NeuroD1 |
| CD195 Phospho (Ser349) | GDAP1L1 | NeuroD2 |
| CD196 | GDF15 | Neurofibromin |
| CD197 | Gelsolin | Neurofilament Heavy Protein |
| CD198 | Gemin1 | Neurofilament Medium Protein |
| CD199 | Gephyrin | Neurogenin 2 |
| CD1a | GFAP | Neurokinin 1 Receptor |
| CD1b | GFP | Neuron Specific Enolase |
| CD1b/c | GILZ | Neuronal Growth Factor Receptor |
| CD1c | GIMAP4 | Neurotensin Receptor 1 |
| CD1d | GIPR | NFÎºB p50/p105 |
| CD1d [alpha]GalCer | GIT2 | NFÎºB p65 (pS536) |
| Complex | | |
| CD2 | GITRL | NFATc1 |
| CD20 | GLAST | NFÎºB p50 |
| CD200 | Gli1 | NFÎºB p50/p105 |
| CD200R | Glial Fibrilary Acidic Protein | NFÎºB p52/p100 |
| CD200R3 | Glicentin | NFÎºB p65 |
| CD201 | GLIPR1L1 | NFÎºB p65 (pS529) |
| CD202b | Glucagon | NG2 |
| CD203a | Glucocorticoid Receptor | NGF |
| CD203c | Glucocorticoid Receptor alpha | Nhedc2 |
| CD204 | Glucose 1 Dehydrogenase | NHERF1 |
| CD205 | Glucose 6 Phosphate | Nicastrin |
| | Isomerase | |
| CD206 | GLUH1 | Ninein |
| CD207 | GLUT1 | Nitrotyrosine |
| CD208 | GLUT2 | NKG2A/C/E |
| CD209 | GLUT4 | NKG2AB6 |
| CD209b | GLUT5 | NKp80 |
| CD21 | Glutamate receptor 2 | NKX3.1 |
| CD21/CD35 | Glutamate receptor 2/3 | NM23A |
| CD210 | Glutamate receptor 3 | NMDA Receptor 2A |
| CD212 | Glutamate receptor 4 | NMDA Receptor 2B |
| CD213al | Glutaminase | NMDE2 |
| CD213a2 | Glutamine Synthetase | NMDZ1 |
| CD217 | Glutaredoxin 2 | NMNA2 |
| CD218a | Glutathione NEM | nMyc |
| CD22 | Glutathione NEW | nNOS |
| CD22 (pY822) | Glutathione Peroxidase 1 | NNTM |
| CD22.2 | Glutathione Peroxidase 4 | Nociceptin |
| CD220 | Glutathione Reductase | Nod2 |
| CD220[alpha] | Glutathione S Transferase | Nodal |
| | [theta]2 | |
| CD221 | Glutathione S Transferase Îº1 | Noggin |
| CD221 (pY1131) | Glutathione S Transferase Î¼ | NONO |
| CD222 | Glutathione Synthetase | Nonspecific Cytotoxic Cells |
| CD223 | Glycogen synthase 1 | Notch1 |
| CD224 | Glycoprotein IX | Notch2 |
| CD226 | Glycoprotein VI | Notch3 |
| CD227 | GM-CSF | Notch4 |
| CD229 | GM130 | NOX2 |
| CD229.1 | GM3.2 | NOX4 |
| CD23 | GNB2 | NOXA2 |
| CD230 | GNB2L1 | NPC |
| CD231 | GNLY | NPM-ALK |
| CD233 | GNMT | NPM/B23 Phospho (Thr199) |
| CD234 | GnRHR | NPM/B23 Phospho |
| | | (Thr234/Thr237) |
| CD235a | Golgi Protein (58K) | NPY5R |
| CD235ab | Golgi Zone | NQO1 |
| CD236 | GOLM1 | NR2E1 |
| CD239 | GOLPH2 | NRC2C |
| CD24 | GOSR1 | Nrf2 |
| CD240CE | gp340 | NRG3 |
| CD240DCE | gp49R | NSPA/B |
| CD243 | GPA33 | NTAL |
| CD244 | GPCR5C | NTF97 |
| CD244.1 | GPR-120 | Nucleolin |
| CD244.2 | GPR-143 | Nucleolin Phospho (Thr76/Thr84) |
| CD245 | GPR-151 | Nucleophosmin |
| CD246 | GPR-18 | NUDC |
| CD247 | GPR-30 | NUMA1 |
| CD247 (pY142) | GPR-40 | Nur77 |
| CD249 | GPR-48 | O acetyl GD3 |
| CD25 | GPR-49 | Oct2 |
| CD252 | GPR-50 | Oct3/4 |
| CD253 | GPR-56 | Oct3/4A |
| CD254 | GPR-73A | Oct4 |
| CD255 | GPR-73B | ODAG |
| CD256 | GPR-77 | OGDH |
| CD257 | GPR-83 | OLIG1 |
| CD258 | GPR-86 | OLIG2 |
| CD26 | GPR-C5C | Oligodendrocyte Marker |
| CD261 | GPR-C5D | Oligodendrocyte Marker O1 |
| CD262 | Granulin | Oligodendrocyte Marker O4 |
| CD263 | Granulysin | Oncostatin M Receptor |
| CD264 | Granzyme A | Orai1 |
| CD265 | Granzyme B | OSCAR |
| CD266 | Granzyme K | OSR1 |
| CD267 | GRAP2 | Osteonectin |
| CD268 | GRASP1 | Osteopontin |
| CD269 | GRASP65 | Osteoprotegerin |
| CD27 | GRB2 | Otx2 |
| CD270 | GRB7 | OVA (SIINFEKL) H-2Kb |
| CD271 | GRHPR | Oval Cell Marker |
| CD272 | GRIM19 | Ovalbumin |
| CD273 | GRK1 | Ovarian Carcinoma-associated |
| | | Antigen |
| CD274 | GRK2 | OX-62 |
| CD275 | GRK3 | p110[delta] |
| CD276 | GRK5 | p120 Catenin |
| CD277 | GRK6 | p120 Catenin (pS268) |
| CD278 | Growth hormone receptor | p120 Catenin (pS288) |
| CD279 | GRP170 | p120 Catenin (pS879) |
| CD28 | GRP94 | p120 Catenin (pT310) |
| CD280 | GSC | p120 Catenin (pT916) |
| CD281 | GSK3[alpha] | p120 Catenin (pY228) |
| CD282 | GSK3[alpha]/[beta] | p13 |
| CD283 | GSK3[beta] | p130 |
| CD284 | GSPT2 | p130 Cas |
| CD284/MD2 Complex | GST | p130 Cas (pY249) |
| CD286 | GST Epitope Tag | p14ARF |
| CD289 | GSTA4 | p150,95 |
| CD29 | GTF2D1 | p19ARF |
| CD290 | GTPase HRAS | p21 |
| CD294 | GTPBP4 | p22phox |
| CD298 | Guanylate kinase | p23 |
| CD299 | H-2 | p27Kip1 |
| CD2a | H-2.m31 | P2RX4 |
| CD3 | H-2Db | P2RY8 |
| CD3/CD44 | H-2Dd | P2X3 |
| CD30 | H-2Kd | P2X7 |
| CD300 | H2-M | P2Y6 |
| CD300a | H2-M3 | p34Cdc-2 |
| CD300e | H2A.X | p38 |
| CD300f | H2A.X Phospho (Ser139) | p38 MAPK (pT180/pY182) |
| CD301 | H2A1J | p400 |
| CD303 | H60 | p53 |
| CD303a | HA tag | p53 Acetylated (Lys305) |
| CD304 | HADHA | p53 Acetylated (Lys382) |
| CD305 | HADHA/HADHB | p53 Phospho (Ser15) |
| CD307d | HADHB | p53 Phospho (Ser37) |
| CD309 | HADHSC | p53 Phospho (Ser392) |
| CD31 | HAND1 | p53BP1 (Ser1778) |
| CD310 | HAO1 | p57Kip2 |
| CD312 | Haptoglobin | p60 CAF1 |
| CD314 | HARS | p62 |
| CD314 (activating) | HARS2 | p63 |
| CD314 (blocking) | HBF | p63 (TA) |
| CD317 | hCG[alpha] | p70 S6 Kinase [beta] |
| CD318 | hCG[beta] | p90 Rsk |
| CD319 | hCG[beta]4 | p90 Rsk Phospho (Thr368/Ser372) |
| CD32 | HCN4 | p95 NBS1 |
| CD321 | HDAC1 | p97 |
| CD323 | HDAC10 | PA28[gamma] |
| CD324 | HDAC2 | PABP1 |
| CD325 | HDAC3 | PABP2 |
| CD326 | HDAC4 | PABPN1 |
| CD328 | HDAC6 | PAC1 |
| CD329 | HDAC9 | PAD2 |
| CD32B | HDHD1A | PAG1 |
| CD33 | HDHD2 | PAK1 |
| CD334 | HDJ2 | PAK2 |
| CD335 | HDLBP | PAK3 |
| CD336 | HE4 | pan Actin |
| CD337 | HEC1 | pan Macrophage |
| CD338 | HEF1 | Panendothelial Cell Antigen |
| CD339 | Helios | PAR1 |
| CD34 | Hematopoiesis related | Parainfluenza Virus type 1 |
| | Macrophage | |
| CD340 | Hematopoietic Lineage | Parainfluenza Virus type 2 |
| | Cocktail | |
| CD344 | Hematopoietic Progenitor Cell | Parainfluenza Virus type 3 |
| CD349 | Hemoglobin | PARC |
| CD35 | Hemoglobin F | PARD3 |
| CD351 | Hemoglobin subunit [alpha] | PARK7/DJ1 |
| CD354 | Hepatitis B Virus | PARP, Cleaved Form |
| CD357 | Hepatitis B Virus Core | PARP16 |
| | Antigen | |
| CD358 | Hepatitis B Virus E Antigen | PARP4 |
| CD36 | Hepatitis B Virus Surface | PARVA |
| | Antigen (Ad/Ay) | |
| CD360 | Hepatitis C Virus | Pax2 |
| CD361 | Hepatitis C Virus Core | Pax5 |
| | Antigen | |
| CD36L1 | Hepatitis C Virus NS4 | Pax6 |
| CD37 | Hepsin | Pax7 |
| CD38 | HER2 | Pax8 |
| CD39 | HER3 | Pax9 |
| CD39L4 | HER4 | Paxillin |
| CD3D | Hes1 | Paxillin Phospho (Tyr118) |
| CD3G | Hexokinase | Paxillin Phospho (Tyr31) |
| CD3[gamma] | Hexokinase1 | PBEF |
| CD3[delta] | Hexokinase2 | PBK |
| CD3[epsilon] | HFE1 | PBP |
| CD3[epsilon] (CD3 | HGF | PBR |
| Molecular Complex) | | |
| CD4 | HGFA Inhibitor 1 | PBX3 |
| CD4 (domain 1) | HHEX | PCB |
| CD4 (domain 2) | HHV8 GPCR | PCNA |
| CD4 v4 | HIBCH | PCYT1A |
| CD40 | HID1 | PD-1H |
| CD40bp | HIF-1[alpha] | PD-ECGF |
| CD41 | HIF-2[alpha] | PDC-TREM |
| CD41/CD61 | HIF1AN | PDCD4 |
| CD41a | HINT1 | PDCD6 |
| CD41b | HIP2 | PDE3B |
| CD42a | HIPK2 | PDECGF |
| CD42b | Hippocalcin | PDGF-AA |
| CD42d | Histamine H3 Receptor | PDI |
| CD43 | Histocytes | PDK1 |
| CD44 | Histone H1 | PDK2 |
| CD44 (v3) | Histone H1.0 | PDPK1 |
| CD44 (v4) | Histone H2A | PDPK1 (pS241) |
| CD44 (v5) | Histone H2B | PDX1 |
| CD44 (v6) | Histone H2B type 1B | PDZK1 |
| CD44 (v7) | Histone H3 | PE |
| CD44.2 | Histone H3 Phospho (Ser10) | PECR |
| CD44std | Histone H3 Phospho (Ser28) | PEI-Transferrinfection |
| CD44v6 | Histone H3.3 | Pellino 1 |
| CD44var (v10) | Histone H4 | Pentraxin 3 |
| CD44var (v3) | HIV1 Core Antigen | PEPD |
| CD44var (v3-v10) | HIV1 p17 | Perforin |
| CD44var (v4) | HIV1 p24 | Peroxiredoxin 1 |
| CD44var (v5) | HIV1 p55/p17 | Peroxiredoxin 2 |
| CD44var (v6) | HIV1 tat | Peroxiredoxin 6 |
| CD44var (v7) | HL60 | PEX5 |
| CD44var (v7-v8) | HLA Class I | PF4 |
| CD45 | HLA-2Kb/2Db | PGC1[alpha] |
| CD45.1 | HLA-2kb/2Dd | PGIS |
| CD45.2 | HLA-A | PGP9.5 |
| CD45R | HLA-A/B/C | PGRP-Ia |
| CD45RA | HLA-A1/A11/A26 | PGRP-S |
| CD45RB | HLA-A1/A36 | PHD1 |
| CD45RC | HLA-A10/A11 | PHD2 |
| CD45RO | HLA-A10/A28/B75 | Phosphatidylserine |
| CD46 | HLA-A10/B62/B71 | Phospho SHIP |
| CD47 | HLA-A11 | Phospholipase A2 activator |
| | | protein (PLAP) |
| CD48 | HLA-A2 | Phospholipase C [beta]3 |
| CD49a | HLA-A2/A25/A32 | Phospholipase C [gamma]1 |
| CD49a/CD29 | HLA-A2/A28 | Phospholipase D1 |
| CD49b | HLA-A2/A3/A29 | Phosphoserine/threonine/tyrosine |
| CD49b/CD29 | HLA-A2/A69 | Phosphotyrosine |
| CD49b/CD61 | HLA-A2/B17 | PI 3 Kinase catalytic subunit |
| | | [alpha] |
| CD49c | HLA-A2/B5 | PI 3 Kinase catalytic subunit |
| | | [gamma] |
| CD49d | HLA-A2/B57 | PI 3 Kinase p110 [beta] |
| CD49d/CD29 | HLA-A23/A24 | PI 3 Kinase p110 [delta] |
| CD49e | HLA-A24/A11/A2403 | PI 3 Kinase p150 |
| CD49e/CD29 | HLA-A25 | PI 3 Kinase p85 [alpha] |
| CD49f | HLA-A25/A26 | PI 4 kinase [beta] |
| CD49f/CD29 | HLA-A25/A26/A34 | PIAS1 |
| CD4[alpha] | HLA-A25/A32 | PIAS3 |
| CD5 | HLA-A26/A34/B71/B62 | PICK1 |
| CD5.1 | HLA-A29 | PIM1 |
| CD5.2 | HLA-A3 | PIM2 |
| CD5.6 | HLA-A30/A31 | Pin1 |
| CD50 | HLA-A33/B8 | PINK1 |
| CD51 | HLA-A34/B71/A26 | PIP5K2[alpha] |
| CD51/61 | HLA-A9 | PIP5KI[gamma] |
| CD52 | HLA-A9/A25/A32 | PIR-A/B |
| CD53 | HLA-A9/A32/B13 | Pirh2 |
| CD54 | HLA-B | PIST |
| CD55 | HLA-B12 | PiTX3 |
| CD56 | HLA-B13/B62/B15 | PIWIL2 |
| CD57 | HLA-B14 | PKA RII[alpha] (pS99) |
| CD58 | HLA-B17 | PKA RII[beta] (pS114) |
| CD59 | HLA-B17/B35/B44 | PKA2[beta] |
| CD59a | HLA-B21/B70/B55 | PKAR2 |
| CD6 | HLA-B27/B44/B47 | PKA[gamma] |
| CD60b | HLA-B35/B57/B75/B77 | PKC |
| CD61 | HLA-B44/B75/B17 | PKCq |
| CD62E | HLA-B48/B60 | PKC[alpha] |
| CD62L | HLA-B5/B49/B56 | PKC[alpha] (pT497) |
| CD62P | HLA-B7 | PKC[alpha] (pT638) |
| CD63 | HLA-B8 | PKC[beta] |
| CD64 | HLA-B8/B14 | PKC[beta]2 |
| CD64 a, b alloantigens | HLA-BC | PKC[gamma] |
| CD64.1 | HLA-Bw4/A9/A32 | PKC[delta] |
| CD65 | HLA-Bw6 | PKC[epsilon] |
| CD65s (CD65 sialylated) | HLA-Bw6/B77 | PKC[zeta] |
| CD66 | HLA-class I free chain | PKC[theta] |
| CD66a | HLA-D | PKC[eta] |
| CD66a/b/c/e | HLA-DM | PKN |
| CD66a/c/d | HLA-DO | PKN2 |
| CD66a/c/d/e | HLA-DP | PKR |
| CD66a/c/e | HLA-DQ | PKX1 |
| CD66a/e | HLA-DQ/DR | PLA2G1B |
| CD66b | HLA-DQ1/DQ3 | Placental alkaline phosphatase |
| CD66c | HLA-DQ1/DR7 | Placental Protein 14 |
| CD66c/e | HLA-DQ3 | Plakophilin 3 |
| CD66e | HLA-DQ6 | Plastin L |
| CD66f | HLA-DQ7 | Platelet |
| CD68 | HLA-DQA1 | PLAU |
| CD69 | HLA-DQB1 | PLC[gamma]1 |
| CD7 | HLA-DQw1 | PLC[gamma]1 (pY783) |
| CD70 | HLA-DR | PLC[gamma]2 |
| CD70b | HLA-DR/DP | PLC[gamma]2 (pY759) |
| CD71 | HLA-DR/DP/DQ | Plectin |
| CD72 | HLA-DR1 | Pleiotrophin |
| CD72 a, b, c alloantigens | HLA-DR11 | PlexinA1 |
| CD72 b, c alloantigens | HLA-DR3/DR6 | PlexinB2 |
| CD72.1 | HLA-DR4 | PLGF |
| CD73 | HLA-DR7 | PLK1 |
| CD74 | HLA-DR7/DR[beta] | PLK1 Phospho (Thr210) |
| CD75 | HLA-DR8/DR12 | PLK4 |
| CD77 | HLA-DR9 | PLSCR1 |
| CD78 | HLA-DRA | PLVAP |
| CD79a | HLA-DR[beta] | PLZF |
| CD79b | HLA-DR[beta]3 | PMCA(1-4) |
| CD8 | HLA-E | PMCA4 |
| CD80 | HLA-G | PMEL17/SILV |
| CD81 | HLCS | PMN |
| CD82 | HLF | PMP70 |
| CD83 | HLXB9 | PMS2 |
| CD84 | HMG14 | PNAd |
| CD85 | HMG17 | PNPH |
| CD85a | HMG4 | Podocalyxin |
| CD85d | HMGB1 | Podoplanin |
| CD85g | HMGB2 | POKEMON |
| CD85h | HMOX1 | Polyhistidine Tag |
| CD85j | HMOX2 | PON1 |
| CD85k | HNF4[alpha] | PON3 |
| CD86 | hnRNPA1 | PP2A[alpha] |
| CD87 | hnRNPC1/C2 | PP2A[alpha][beta] |
| CD88 | hnRNPD | PPM1A |
| CD89 | hnRNPK | PPP1A |
| CD8[alpha] | hnRNPL | PPP5C |
| CD8[alpha].1 | hnRNPU | PPP6C |
| CD8[alpha].2 | hnRNPUL1 | PR3 |
| CD8[beta] | Homing Receptor | PRA1 |
| CD9 | HOXB4 | PRC1 |
| CD90.1 | HOXB5 | Pre-BCR |
| CD90.2 | HP1[alpha] | Pre-T Cell Receptor |
| | | [alpha] Chain |
| CD90.9 | HPal | Prealbumin |
| CD91 | HPa2 | Presenilin1 |
| CD91[alpha] | HPD | Presenilin2 |
| CD91[beta] | HPd1 | Prion protein PrP |
| CD93 | HPd2 | PRKRA |
| CD94 | HPi1 | PRLR |
| CD95 | HPi2 | PRMT1 |
| CD96 | HPi3 | PRMT5 |
| CD97 | HPi4 | pro Relaxin 1/2 |
| CD98 | HPR1 | pro Relaxin 2 |
| CD98hc | HPRT1 | Profilin1 |
| CD99 | HPV16 E1/E4 | Progesterone Receptor |
| CD99R | HPx1 | Prohibitin |
| Coagulation Factor VII | DSCAM-L1 | Eph Receptor A5 |
| CXCL1/2/3 | FLRT1 | Ephrin B2 |
| DDR2 | Frizzled-6 | CD316 |
| DPCR1 | Glypican1 | Kremen1 |
| Dipeptidyl peptidase 6 | IGSF4B | Eph Receptor B1 |
| Epithelial membrane | IL-1R9 | PlexinB3 |
| protein 3 | | |
| Endoglycan | BAZ2B | DMBT1 |
| Calgranulin C | BRD4 | FcRn |
| FATP2 | Kell | LIMPII |
| FATP5 | Kremen2 | MUCDHL |
| FcRLB | LAX1 | Patched1 |
| GLP-2R | CD85c | SLC39A4 |
| GLUT3 | MIF | IGSF4A |
| Glypican6 | Neprilysin2 | PRAT4B |
| GPR-22 | OBCAM | HHV8-ORF74 |
| GPR-37 | PlexinC1 | 4E-BP1 Phospho (Thr36/45) |
| GPR-37L1 | RGM-B | 4E-BP1 Phospho (Thr69) |
| INSRR | Wilmsâ€ ™ Tumor protein 1 | DCAR1 |
| LINGO1 | Xg | Von Hippel-Lindau |
| LINGO2 | DCBLD2 | Isotype Control |
| mGluR2 | ASAM | Granzyme M |
| mGluR7 | Desmocollin1 | REA Isotype Control |
| MMP25 | Frizzled-3 | CD300LG |
| Neuromedin B Receptor | MMP24 | MR1 |
| NRAGE | TOR | CD327 |
| Osteoactivin | WNT3a | B7-H6 |
| Porimin | Glypican5 | CLEC4G |
| Prokineticin Receptor 1 | Jagged1/Jagged2 | BATF3 |
| Prominin2 | Pax3 | IL-38 |
| Semaphorin 3A | CELSR2 | Monocarboxylic Acid |
| | | Transporter 1 |
| SLAP-130 | Cyclin D1/D2 | MC5R |
| Somatostatin Receptor 5 | PlexinA2 | TCF7 |
| SCARF1 | TAFA5 | TM4SF1 |
| STAMP2 | FR4 | GPR-49 (CRL Region) |
| TAFA3 | CD315 | CD156a |
| TAFA4 | NKG2I | ADAM33 |
| TM4SF18 | RAMP2 | ADAMTS13 |
| Tuberous Sclerosis 1 | TNFRH3 | CCL16 |
| TCF8 | Biotin | CXCL17 |
| CMG2 | GPVI | Deltex1 |
| IL-17D Receptor | MS4A4B | FBXO15 |
| Macrophage Stimulating | PIR-B | GPR34 |
| Protein Receptor | | |
| Siglec-11 | Semaphorin 4F | GPRC5A |
| Syndecan3 | IL-1F6 | Proinsulin |
| TGF-[beta]R3 | CD39L3 | JAK1 |
| CD85e | Contactin 3 | MEP1A |
| SOX7 | CLEC4B | Hypocretin receptor 2 |
| Activin A Receptor | MC3R | p70S6K |
| Type IA | | |
| Carbohydrate | PGRP-L | RAE-1[epsilon] |
| Sulfotransferase 15 | | |
| CD300b | PLET1 | STRA6 |
| CELSR3 | ADAM9 | Fc[gamma]RIIA |
| Coagulation Factor II | AMIGO3 | Insulin R/IGF-I R |
| | | Heterotetramer |
| DC-SCRIPT | CD99-L2 | SPARCL1 |
| CD79[alpha]cy | CD92 | XBP1 |
| Prokineticin 1 | SULT1A1 | XBP1 (COOH terminus) |
| Prokineticin 2 | SULT1A3/SULT1A4 | XBPs |
| Prolactin | SULT1C2 | XCL1 |
| ProMBP1 | SULT2A1 | XIAP |
| Prostaglandin D2 Receptor | SUMO1 | XPC |
| Prostaglandin | SUMO2 | XPNPEP3 |
| dehydrogenase 1 | | |
| Prostaglandin E Receptor | SUMO3 | XRCC2 |
| EP3 | | |
| Prostate Cell Surface | SUN1 | XTP4 |
| Antigen | | |
| Prostate Specific Antigen | Suppressor of Fused | YAP1 |
| Prostatic Acid Phosphatase | SUPT16H | YB1 |
| Proteasome 20S C2 | Survivin | YES1 |
| Proteasome 20S [alpha]2 | Survivin Phospho (Thr34) | YY1 |
| Proteasome 20S [alpha]3 | SV40 Large T and Small t | ZAP-70 |
| | Antigens | |
| Proteasome 20S [alpha]5 | SWC1a | ZAP-70 (pY292) |
| Proteasome 20S [alpha]6 | SWC6 | ZAP-70 (pY319) |
| Proteasome 20S [alpha]7 | SYBL1 | ZAP-70 (pY319)/Syk (pY352) |
| Proteasome | Syk | ZBP-1 |
| 20S[alpha]1/2/3/5/6/7 | | |
| Protein A | Syk (pY348) | ZIPK |
| Protein G | Synapsin I | ZO-1 (Mid) |
| Protein Kinase D2 | Synapsin II | ZONAB (Mid) |
| Protein Phosphatase | Synaptojanin2 | Zyxin |
| 1[beta] | | |
| Protein phosphotase | Synaptophysin | IL-33R |
| inhibitor 1 | | |
| Protein S | Syndecan4 | Globo H |
| Proteinase Activated | SynGAP | CCL8 |
| Receptor 4 | | |
| Prothrombin | Synip | Siglec-G |
| PSA-NCAM | Syntaxin | CD307e |
| PSD95 | Syntaxin6 | CLEC6 |
| Pseudomonas Aeruginosa | Syntrophin | Snail1 |
| PSMA | SYWC | SMAD1 (pS463/pS465)/ |
| | | SMAD8 (pS465/pS467) |
| PSMD14 | T cells (pan reactive) | SMAD2 (pS465/pS467)/ |
| | | SMAD3 (pS423/pS425) |
| Psoriasin | T Lymphocytes | GSK-3[beta] (pY216) |
| PTAFR | T- and B-Cell Activation | NKX6.1 |
| | Antigen | |
| PTBP1 | T7 tag | FAK (pY397) |
| PTEN | TAB1 | Btk (pY223)/Itk (pY180) |
| PTGER2 | TACE | ERK3 |
| PTGER4 | TACI | CD276[beta] |
| PTHLH | TAF172 | MCP-3 |
| PTK7 | TAF250 | FcÂμR |
| PTP1B | TAG72 | CD238 |
| PTP4A2 | Talin1 | beta2 Microglobulin [b, c] |
| PTPS | Talin2 | Nucleostemin |
| PTPÎ¼ | Tamm Horsfall (Uromucoid) | GPR-49 (Central LRR) |
| PTRH2 | TANK1 | GPR-49 (N-Terminal) |
| PU.1 | TAP1 | Phospholipase C [beta]4 |
| PU60 | TAP2 | coilin |
| PUMA | TARDBP | HNF1[beta] |
| PUMA[gamma] | TARP | Trinitrophenal |
| Pumilio1 | Tartrate-resistant acid | Annexin VII |
| | phosphatase | |
| Pumilio2 | TAS1R1 | CD301a |
| PXR | Tau | CD301b |
| PYCARD | TBA1B | mTOR (pS2448) |
| Pygopus2 | Tbet | PI16 |
| Pyk2 | TBK1 (pS172) | MSC (W5C5) |
| Pyk2 (pY402) | TBX1 | LAMP5 |
| Pyruvate Dehydrogenase | TC10 | GPR-19 |
| E1[alpha] | | |
| Pyruvate Dehydrogenase | TCF3 | FPRL2 |
| E2 | | |
| Pyruvate Dehydrogenase | TCF7L1 | CXCL5 |
| E2/E3bp | | |
| q2 | TCF7L2 | PAR2 |
| Qa1(b) | TCL1 | PDGF-R[alpha] |
| Qa2 | TCP1[alpha] | ULBP6 |
| RAB11A | TCP1[beta] | ULBP2/5/6 |
| RAB25 | TCR | IL-17B Receptor |
| RAB27A | TCR DO11.10 | ULBP3 |
| RAB4 | TCR HY | Arginase 1 |
| RAB5a | TCR V[alpha]11 | Alkaline Phosphatase |
| RAB9 | TCR V[alpha]11.1/11.2b, d | ULBP3 |
| Rac1 | TCR V[alpha]2 | TrkB |
| Rac1/Cdc42 | TCR V[alpha]24 | Osteocalcin |
| RAD17 | TCR V[alpha]24-J[alpha]18 | IL-22R[alpha]1 |
| RAD17 Phospho (Ser645) | TCR V[alpha]3.2 | APJ |
| RAD23A | TCR V[alpha]3.2b, c | IFN-[alpha]/[beta] |
| | | Receptor Subunit 2 |
| RAD51 | TCR V[alpha]7.2 | FGFR3 |
| RAD54 | TCR V[alpha]8 | SR-A1 |
| RAD9A | TCR V[alpha]8.3 | Rae-1 (pan) |
| Radixin | TCR V[beta]1 | CXCL12 |
| RAE-1[gamma] | TCR V[beta]10a | TREM2 |
| RAE-1[delta] | TCR V[beta]10b | Brachyury |
| RAF1 | TCR V[beta]11 | CLEC5A |
| RAGE | TCR V[beta]12 | Integrin [alpha]7 |
| RAIDD | TCR V[beta]12b | Mer |
| Rainbow Trout Ig | TCR V[beta]13 | XCR1 |
| RalBP1 | TCR V[beta]13.1 | AML2 |
| RanBP9 | TCR V[beta]13.2 | von Willebrands factor A2 |
| RanGAP1 | TCR V[beta]13.6 | MMP7 |
| RAP1A/RAP1B | TCR V[beta]14 | GLP-1R |
| RAP1GAP | TCR V[beta]16 | FR1 |
| Raptor | TCR V[beta]17 | IL-1RAcP |
| RAR[alpha] | TCR V[beta]17[alpha] | Claudin-6 |
| RAS | TCR V[beta]18 | Leptin Receptor |
| RASGAP | TCR V[beta]2 | Caherin 6 |
| RASGRF1 | TCR V[beta]20 | IL-1R type II |
| RASSF1A | TCR V[beta]21.3 | Nectin4 |
| Rb | TCR V[beta]22 | Delta like protein 3 |
| Rb (a.a. 332-344) | TCR V[beta]23 | ChemR23 |
| Rb (pS780) | TCR V[beta]3 | GPR-39 |
| Rb (pS807/pS811) | TCR V[beta]4 | CD158b2 |
| RbAp46 | TCR V[beta]5 | IL-10R[alpha] |
| RbAp48 | TCR V[beta]5.1 | LRIG1 |
| RBC | TCR V[beta]5.1/5.2 | Neuropilin2 |
| RBC (Polyclonal Rabbit) | TCR V[beta]5.2 | IL-10R[beta] |
| RBM35A | TCR V[beta]5.3 | IL-18R[beta] |
| RBP4 | TCR V[beta]6 | GPR-44 |
| RBX1 | TCR V[beta]7 | Eph Receptor B2 |
| RCC1 | TCR V[beta]7.1 | Glypican3 |
| RcRL6 | TCR V[beta]7.2 | IFN-[gamma]R2 |
| Red Blood Cell | TCR V[beta]8 | IL-17C Receptor |
| Relaxin 1 | TCR V[beta]8.1/8.2 | BMPR1B |
| Relaxin 1/2 | TCR V[beta]8.2 | IL-31RA |
| Relaxin 2 | TCR V[beta]8.2/8.3 | OCIL |
| RelB | TCR V[beta]8.2/8.4 | Frizzled-7 |
| RELM[beta] | TCR V[beta]8.3 | IL-26 |
| RELT | TCR V[beta]8.5 | GPR-15 |
| Renin | TCR V[beta]9 | PlexinD1 |
| RENT1 | TCR V[gamma]1.1 | CD158 |
| Reptin | TCR | FPR1 |
| | V[gamma]1.1/[gamma]1.2 | |
| Repulsive Guidance | TCR V[gamma]2 | HBEGF |
| Molecule C | | |
| Resistin | TCR V[gamma]3 | Vitamin D3 |
| REST | TCR V[gamma]9 | PlexinB1 |
| Ret | TCR V[delta]1 | Somatostatin Receptor 2 |
| Reticular Fibroblasts and | TCR V[delta]2 | OV-6 |
| Reticular Fibres | | |
| Reticulon1A | TCR V[delta]4 | CXCL16 |
| Reticulum Cells | TCR V[delta]6.3/2 | Siglec-E |
| Retinoblastoma 1 | TCR [alpha] | EDG5 |
| RFLAT1 | TCR [alpha][beta] | Ninjurin-1 |
| RFP | TCR [beta] | Integrin [alpha]9 |
| RGS6 | TCR [gamma][delta] | MHC Class II (I-Ed/j/k/p/r/u/v) |
| RGS7 | TCR [zeta] | ThB |
| RGS9 | TCTP | MAP-2 (2a & 2b) |
| RHEB | TdT | IgM Î¼-chain |
| Rho | Tec | MHC Class I (H-2b/p) |
| RhoA | TEF1 | MHC Class I (H-2s/p/q/d/u/r) |
| RHOC | TEM8 | MHC Class I (H-2s/f) |
| RhoGAP | Tenascin C | CDw60 |
| RhoGDI | TER119 | Bad Phospho (Ser112) |
| RIAM | TERF2 | Caspase 3 Cleaved (Asp175) |
| RICTOR | Terminal-Deoxynucleotidyl | Chk1 Phospho (Ser345) |
| | Transferase | |
| RIG1 | TERT | Chk2 Phospho (Thr68) |
| RIP1 | Tetranectin | Cyclin D1 Phospho (Thr286) |
| RIP2 | TFF3 | cFos Phospho (Ser32) |
| Rituximab | TFIIB | FosB |
| RLA DQ | TGF-[beta] | GSK-3[beta] (pSer9) |
| RLA DR | TGF-[beta]1 | Histone H3 Acetylated (Lys9) |
| RNA polymerase II | TGF-[beta]3 | HS1 Phospho (Tyr397) |
| RNA polymerase II CTD | TGF-[beta]R1 | Hsp27 Phospho (Ser82) |
| repeat YSPTSPS | | |
| RNASE-L | TGF-[beta]R2 | ID3 |
| RNASE1 | TGN38 | CD221[beta] |
| RNF144B | TGN46 | Phospho-IRAK4 (Thr345/Ser346) |
| RNF168 | THAP11 | Phospho-cJun (Ser73) |
| RNF36 | THEMIS | S6 (pS240/pS244) |
| RNPEP | Thioredoxin | Syk (pY525/pY526) |
| ROCK1 | Thioredoxin Reductase 1 | C23 |
| ROR1 | ThPOK | Hemoglobin [beta] |
| ROR2 | Thrombin Receptor | CD221[alpha] |
| ROR[alpha] | Thrombocyte | p27 |
| ROR[gamma] | Thrombospondin | cJun Phospho (Ser63) |
| ROS | Thymidine Kinase 1 | PPAR[gamma] |
| RPA32/RPA2 | Thyroglobulin | ENPP1 |
| RPA70 | TIA-1 | PILR[alpha] |
| RPS6 | TIAM2 | PILR[beta] |
| RSF1 | Tie1 | Twist1 |
| RSK1 p90 | Tie2 (pY1102) | Cadherin M |
| RSK2 | Tie2 (pY992) | CD302 |
| RSK3 | TIF1[beta] Phospho (Ser473) | CD66d |
| RSK4 | TIGIT | CLEC14A |
| RT1A | Tim1 | CD242 |
| RT1Aa | Tim2 | Syndecan2 |
| RT1Aa, b | Tim3 | IL-32[alpha] |
| RT1Aa, b, l | Tim3 Fc Fusion Protein | CDO |
| RT1Ac | Tim4 | Cryptic |
| RT1Au | Tim50 | Endothelin B Receptor |
| RT1B | Timeless | FR3 |
| RT6.1 | TIMP1 | IGSF3 |
| RT6.2 | TIMP2 | CD85f |
| Ryanodine Receptor | TIP49A | Matriptase |
| RYK | TIRAP | MCEMP1 |
| RyR | TIS11b | mGluR4 |
| S-Tag | TL1A | Stabilin1 |
| S100A1 | TLK1 | Stabilin2 |
| S100A10 | TLR11 | Cadherin 13 |
| S100A13 | TLR12 | GPR-109A |
| S100A4 | CD285 | TSPAN8 |
| S100A6 | TLR7 | Reg1A |
| S100A9 | TLR8 | Cadherin 12 |
| S100[alpha] | TMEFF2 | ECE1 |
| S100[alpha]2 | TMPS2 | FABP5 |
| S100[beta] | TMSA | IGSF4C |
| S6 (pS235/pS236) | TMTSP | Trem-like 1 |
| S6 (pS240) | TNAP | Activin A Receptor Type IIA |
| S6 (pS244) | TNAP3 | ALK7 |
| S6K | TNF-[alpha] | BCAM |
| SAA4 | TNF-[beta] | BLAME |
| Sall4 | TNFR Related Protein | CEACAM4 |
| Salmonella Paratyphi A | TNPO3 | Claudin-3 |
| Salmonella Typhimurium | Tollip | CLP24 |
| Salmonid Ig (H and L | TOMM20 | CRHR1 |
| chain) | | |
| Salmonid Ig (H chain) | TOMM22 | DC-STAMP |
| SAM68 | TOP1 | Eph Receptor B3 |
| SAMD2 | TOP2A | FATP4 |
| SAP | TOP2B | FcRL1 |
| SARA | TORC2 | FcRL2 |
| SATB1 | Torsin A | FcRL3 |
| SATB2 | TOX | FSH-R |
| SC5A5 | TPH1 | Gi24 |
| SC6A4 | TPPP | Histamine H1 Receptor |
| SCAI | TPTE | Neu5Gc |
| SCD1 | TR11B | Lin28A |
| Scramblase1 | TRA-1-60 | IL-33R[alpha] |
| SCY1-like 3 | TRA-1-60R | ATM (pSer1981) |
| SDF1 | TRA-1-81 | Integrin [alpha]8 |
| SDF1[alpha] | TRA-2-49 | Integrin [beta] 7 |
| SDHA | TRA-2-54 | Integrin [beta]8 |
| SDHB | TRADD | CD158k |
| Secretory component | TRAF2 | KOR |
| Securin | TRAF4 | CD85i |
| SELP | TRAF5 | LRIG3 |
| Sema4A | TRAF6 | LRP4 |
| Sema7A | TRAM2 | MMP16 |
| SENP1 | Transferrin | MS4A4A |
| SEPP1 | Transglutaminase | NAALADase-like 2 |
| SERCA2 | Transglutaminase2 | Neuropeptide Y receptor type 1 |
| SerpinB1 | Transketolase | Oncostatin M Receptor [beta] |
| SerpinB2 | TRAP1 | MS4A3 |
| SerpinB6 | TRAPPC2 | PEAR1 |
| Sestrin1 | TRAP[alpha] | PEDF Receptor |
| SFRP2 | Trem-like 2 | PlexinA4 |
| SGK1 | Trem-like 4 | Protocadherin 1 |
| SHC1 | TRIB2 | ROBO2 |
| Shigella Boydii | TRIB3 | ROBO4 |
| SHIP1 | TRIM | EDG8 |
| SHP1 | TRIM25 | Scavenger receptor A5 |
| SHP2 | TRIM29 | Semaphorin 4A |
| SHP2 (pY542) | TRK | Semaphorin 4B |
| SIAH2 | TrkA | Semaphorin 6A |
| SIGIRR | TrkC | Siglec-16 |
| Siglec-10 | Trop2 | Somatostatin Receptor 3 |
| Siglec-8 | Tropomyosin 1 | STING |
| Siglec-9 | TROY | GPBAR1 |
| Siglec-F | TRPC6 | TM4SF4 |
| Siglec-H | TRPM2 | TMEM87A |
| SIK2 | TRPM8 | TSPAN2 |
| SIRT1 | TRX1 | VEGF-R1, 2, 3 |
| SIRT2 | Trypanosoma brucei Major | ADAM15 |
| | Lysosomal Protein | |
| SIRT3 | Trypanosoma brucei procyclin | Calreticulin2 |
| | (EP) | |
| SIRT5 | Trypanosoma congolense | Complement Factor H-related 4 |
| | procyclin | |
| SIT1 | Trypanosoma cruzi LPG | CXCL6 |
| SIX2 | TSC2 Phospho (Ser664) | CD158a/h/b2/f/g |
| SKP1A | TSC2 Phospho (Thr1462) | Ea52-68 peptide bound to I-Ab |
| SLA-DR | TSG101 | HLA-Bw4 |
| Slan | TSHR | ATF1 Phospho (Ser63) |
| SLC1A3 | TSLP | Epiregulin |
| SLC1A7 | TSLP Receptor | FATP1 |
| SLC22A1 | TSPO | Fibromodulin |
| SLC22A5 | TTF1 | Furin |
| SLC26A6 | Tubb3 | Galanin |
| SLC26A7 | Tuberin | IL-11 |
| SLC30A4 | Tubulin [alpha] | CD306 |
| SLC39A11 | Tubulin [alpha]1B | MFG-E8 |
| SLC4A3 | Tubulin [alpha]4a | MINA |
| SLC6A19 | Tubulin [alpha]3E | Oct4A |
| SLC6A6 | Tubulin [alpha]8 | OLIG1, 2, 3 |
| SLC7A10 | Tubulin [beta] | Oncostatin M |
| SLC7A14 | Tubulin [beta] class III | Semaphorin 3E |
| SLC7A3 | Tubulin [beta]4 | Slug |
| SLC7A8 | Tubulin [gamma] | SOX3 |
| SLC8A2 | tumor antigens of epithelial | STYK1 |
| | origin | |
| SLC9A6 | Twist2 | LTBP1 |
| SLP76 | TXNIP | TIMP3 |
| SLP76 (pY128) | TYK2 | VAP-B |
| SM22[alpha] | TYMS | WNT9a |
| SMAC | Tyro3 | 5HT2C |
| SMAC3 | Tyrosinase | AATK |
| SMAD1 | Tyrosine Hydroxylase | ACLP |
| SMAD1 (pS463/465) | UACA | ADAMTS15 |
| SMAD1/5 | UBA52 | alpha 1B Adrenoreceptor |
| SMAD1/9 | UBC9 | APLP1 |
| SMAD2 | UBE2 | Fluorescein/Oregon Green |
| SMAD2/3 (pS465/467) | UBE2L3 | RXR-[beta] |
| DELETE | | |
| SMAD3 | UBE2L6 | L3MBTL3 |
| SMAD4 | UBE2M | CCL1 |
| SMAD5 | UBE2N | PRDM4 |
| SMAD6 | UBF | ACTH |
| SMC1 | UBF1 | PDZ binding kinase |
| SMC1L1 | Ubiquitin | HuC/HuD neuronal protein |
| SMN | UBK63 | TDRD3 |
| Smoothelin | UCH37 | EP300 |
| SMURF2 | UCK | Carbonic Anhydrase VI |
| SNAP25 | UCP2 | Cholecystokinin A Receptor |
| SNX1 | UCP3 | CCL23 |
| SOAT1 | UFM1 | CD1e |
| SOCS1 | ULBP1 | Chondrolectin |
| SOCS2 | ULBP2 | Chordin-Like 2 |
| SOCS3 | ULBP4 | Claudin-10b |
| SOCS6 | ULK3 | Claudin-11 |
| SOD2 | UNC5A | Claudin-12 |
| Sodium Potassium ATPase | UNC5B | Claudin-17 |
| Sonic Hedgehog | UNG | CLEC2A |
| Sortilin | uPA | Spi-B |
| SOSC3 | UQCRC1 | TRAM |
| SOX1 | UQCRC2 | Carboxypeptidase E |
| SOX10 | Urm1 | Islet Cell Autoantigen 1 |
| SOX17 | URP2 | Patched2 |
| SOX18 | USF1 | ST8SIA2 |
| SOX2 | USP11 | AML1 (pS249) |
| SOX2 (COOH terminus) | USP13 | AMPK[beta]1 (pS182) |
| SOX2 (NH2 terminus) | USP22 | BRF1/2 |
| SOX9 | USP28 | Histone H3 Phospho (Thr11) |
| SP-D | USP7 | MEK1 (pT286) |
| Sp1 | UTF1 | MMP16 |
| Sp3 | V5 tag | MNK Phospho (T197/T202) |
| Spectrin [alpha]1 | VAMP5/8 | NUMB |
| SPHK1 | VAP1 | Hsp27 Phospho (Ser78) |
| Spt16 | VASA | PKC[theta] (pT538) |
| Src (pY418) | VASP | SIRT1 (pS47) |
| SREBP1 | VAV1 | ZAP-70 (pY493) |
| ssDNA | VAV2 | ZAP-70 (pY315/pY319) |
| SSEA3 | VAV3 | sRAGE |
| SSEA4 | VDAC1 | mCherry |
| SSEA5 | VEGF | PI 3 Kinase regulatory subunit |
| | | [alpha] |
| SSH3BP1 | VEGF-120 | TIMP4 |
| SSR2 | VEGF-A | SRC |
| SSR5 | VEGF-R1 | ZAP-70 (pT493) |
| SSRP1 | VELIS-3 | TSC2 Phospho (S939) |
| SSX2IP | VGLU1 | RagC |
| Stat1 | Villin | SHIP2 |
| Stat1 (N-Terminus) | Vimentin | MKK4 (pS257) |
| Stat1 (pS727) | Vinculin | CD79a (pY182) |
| Stat1 (pY701) | Viperin | TRAF1 |
| Stat1[alpha] | VIPR1 | EVI1 |
| Stat2 | Vitamin D Binding protein | SRC3 |
| Stat3 | Vitamin D Receptor | SOX11 |
| Stat3 (pS727) | Vitronectin | IL-17F homodimer |
| Stat3 (pY705) | VMAT2 | CCRL1 |
| Stat4 | vMyb/cMyb | FOXP2 |
| Stat4 (pY693) | von Willebrands factor | IFNAR2 |
| Stat5 | VRK1 | REA Control |
| Stat5 (pY694) | VSV-G tag | CD228 |
| Stat5a | WAPL | Muc-13 |
| Stat5b | WASP | P2X7R |
| Stat6 | WC14 | Btk (pY223/Itk (pY180) |
| Stat6 (pY641) | WC15 | CD248 |
| Stathmin/Op18 Phospho | wCD44 | GILT |
| (Ser16) | | |
| Stathmin1 | WIP (pS488) | Recoverin |
| Stefin B | WNT1 | Cardiac Troponin I |
| Stem Cell Factor | WNT16 | PTF1[alpha] |
| STIM1 | WNT2 | NKX2.2 |
| STK3 | WNT5B | HLA-B7/B27 |
| STK33 | WNT6 | Myosin light chain 2a |
| STK39 | WSTF | Myosin light chain 2v |
| STOM | WWOX | Epithelial Antigen |
| STRO1 | Xanthine Oxidase | |

### EXAMPLES

The present invention is further illustrated by reference to the following Examples. However, it should be noted that these Examples, like the embodiments described above, are illustrative and are not to be construed as restricting the scope of the invention in any way.

### Example 1: Generation of Synthetic Polymer Particles

Photomasks for UV lithography were sourced from CADart Services Inc. and were designed using AutoCad (AutoDesk, Inc.). SU-8 photo resist (Microchem, Inc.) was photo crosslinked on 4″ silicon wafers using a collimated UV light source (OAI, Inc.) to create masters for microfluidic device fabrication. PDMS (polydimethylsiloxane, Sigma Aldrich, Inc.) was prepared and formed using standard published methods for soft lithography and microfluidic device fabrication (See, McDonald J C, et al., 2000, Electrophoresis 21:27-40).

Droplets were formed using flow-focusing geometry where two oil channels focus a central stream of aqueous monomer solution to break off droplets in a water-in-oil emulsion. A fluorocarbon-oil (Novec 7500 3M, Inc.) was used as the outer, continuous phase liquid for droplet formation. To stabilize droplets before polymerization, a surfactant was added at 0.5% w/w to the oil phase (ammonium carboxylate salt of Krytox 157 FSH, Dupont). To make the basic polyacrylamide gel particle, a central phase of an aqueous monomer solution containing N-acrylamide (1-20% w/v), a cross-linker (N,N′-bisacrylamide, 0.05-1% w/v), an accelerator, and ammonium persulfate (1% w/v) was used. An accelerator, (N,N,N′,N′-tetramethylethylenediamine (2% vol %) was added to the oil-phase in order to trigger hydrogel particle polymerization after droplet formation.

Several co-monomers were added to the basic gel formulation to add functionality. Allyl-amine provided primary amine groups for secondary labeling after gel formation. We modulated forward scatter by adjusting the refractive index of the gel by adding co-monomers allyl acrylate and allyl methacrylate. Side scattering of the droplets was tuned by adding a colloidal suspension of silica nanoparticles and/or PMMA (poly(methyl methacrylate)) particles (˜100 nm) to the central aqueous phase prior to polymerization.

Stoichiometric multiplexing of the hydrogel particles was achieved by utilizing co-monomers containing chemically orthogonal side groups (amine, carboxyl, maleimide, epoxide, alkyne, etc.) for secondary labeling.

Droplets were formed at an average rate of 5 kHz and were collected in the fluorocarbon oil phase. Polymerization was completed at 50° C. for 30 minutes, and the resulting hydrogel particles were washed from the oil into an aqueous solution.

### Example 2: Generation and Visualization of Synthetic Particles

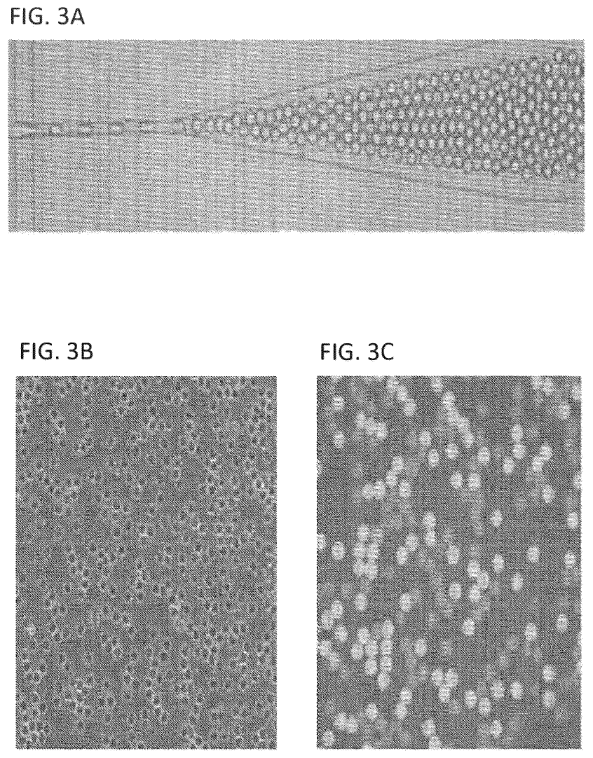

Water containing 5% acrylamide, 0.25% bisacrylamide, 0.05% allyl amine, and 0.1% ammonium persulfate was flowed through a center channel and focused by oil containing 0.1% TEMED through a 10 μm nozzle to produce 10 μm hydrogel particles, shown in FIG. 3A. Following polymerization, the particles were washed in water, shown in FIG. 3B, and conjugated to dyes of interest. The fluorescent hydrogel particles were visualized with fluorescence microscopy, shown in FIG. 3C.

### Example 3: Formation and Functionalization of Porous Synthetic Particles

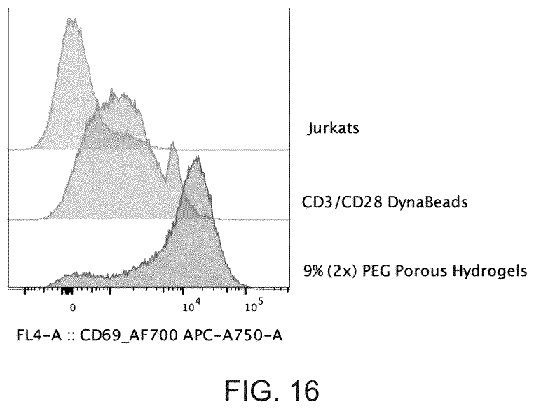

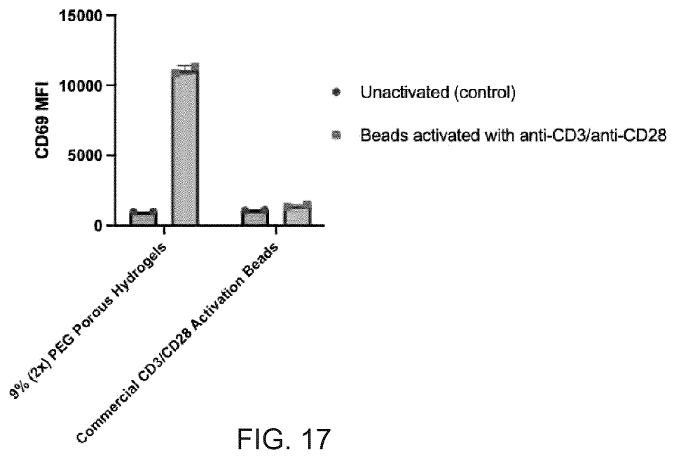

With reference to FIG. 14, to fabricate porous particles, first, an aqueous solution, or continuous phase, of monomers was formed (e.g., acrylamide and bis-acrylamide at 0.62M with the addition of 0.0036M of streptavidin-acrylamide dissolved in a 100 mM pH 7.5 Tris-HCl buffer). An additive (e.g., linear PEG 8000) was added (e.g., at 9 wt %) to the aqueous solution to form a dispersed phase. From the aqueous phase, droplets were formed using a microfluidic polydimethylsiloxane (PDMS) device configured (e.g., using the channels, flow rates, and/or pressures) to control the droplets' form (e.g., having an average droplet diameter of about 20 μm). The droplets were collected, de-gassed, and then cured in the presence of a polymerization agent (e.g., ammonium persulfate at 0.1 wt %). Oil (e.g., 1H, 1H, 2H, 2H-Perfluorooctan-1-ol (PFO)) was added to the cured droplets (e.g., at a 1:1 ratio) to obtain crude particles. The crude particles were washed and purified several times with water to obtain the particles by phase separation. FIG. 15 is a microscopy image of porous particles formed using polyethylene glycol (PEG).

### Example 4: Porous Hydrogel Particles as Immune Response Activators

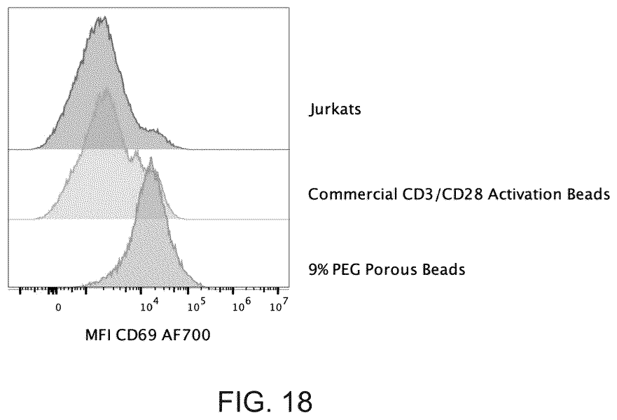

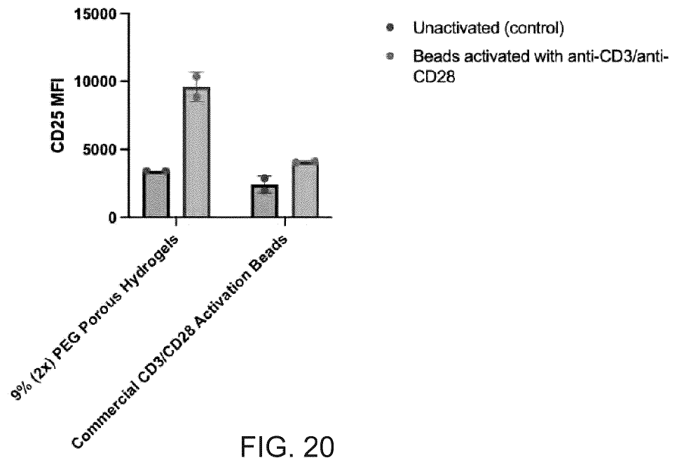

Porous particles generated according to Example 3 were used in immune cell activation assays. With reference to FIG. 16-21, a porous hydrogel particle was fabricated according to the above and the below specifications.

| PEG mw8000 | 0-9% | |
|---|---|---|
| TrisHCl | 100 | mM |
| Acrylamide | 0.62-0.96M | |
| Bis-acrylamide (5% Bis/acrylamide) | 0.62-0.96M | |
| PS100 | 0-1.13% | |
| Streptavidin-acrylamide | 0-0.6 | mg/mL |

| | |
|---|---|
| APS | 0.1-0.2% |

Immune co-stimulatory biomolecules were added to the hydrogel matrix of the porous particles. A set of particles comprising anti-CD3 and anti-CD28 antibodies were produced and tested for T-Cell expansion assay. Other combinations were also tested (e.g., comprising CD19).

Using these porous particles for cell activation showed stronger and more retained TCR engagement and stimulation while removing the magnetic depletion step used in current activation methods.

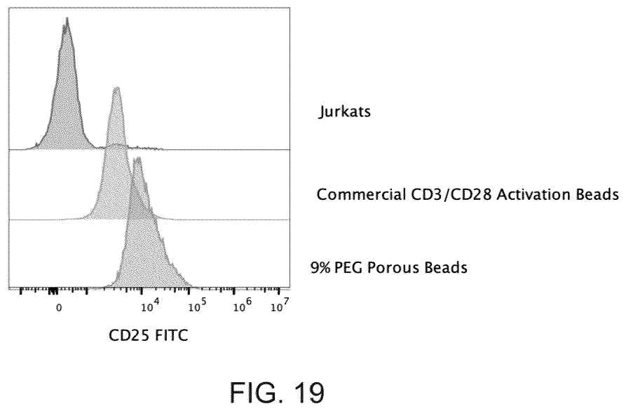

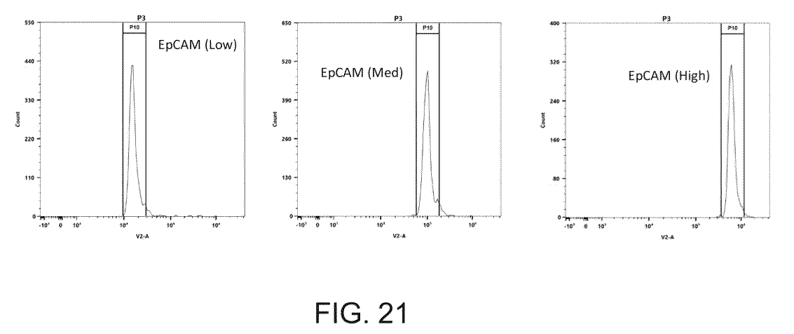

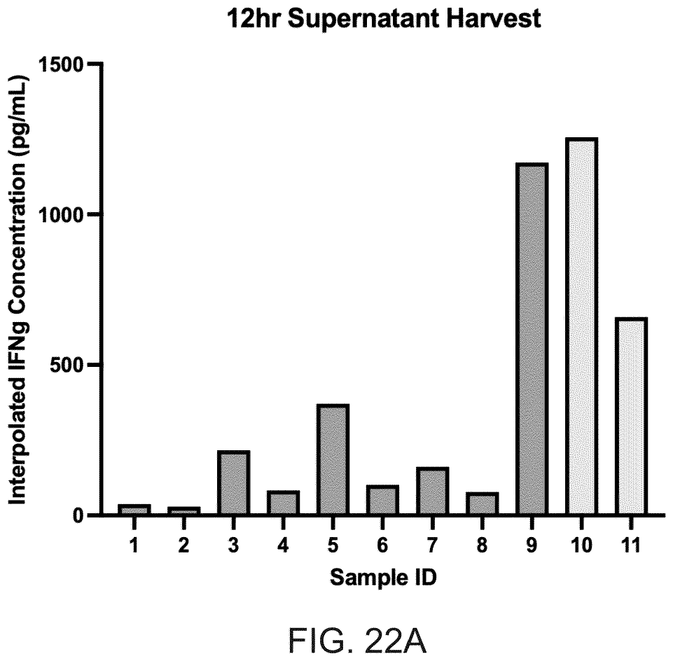

Activation efficiency was measured using early and late-stage T cell activation markers, CD69, as shown in FIGS. 16-18, and CD25, as shown in FIG. 19 and FIG. 20, at various time points post incubation with T cells. For example, FIG. 16 shows early-stage activation was increased in Jurkat samples incubated with 9% PEG porous hydrogel particles compared to Dynabeads™ at 24 hours, as evidenced by upregulation in CD69, an early-stage activation marker. FIG. 17 shows late-stage activation was increased in Jurkat samples incubated with 9% PEG porous hydrogel particles compared to Dynabeads™ at 48 hours, as evidence by sustained activation of CD25, a late-stage T cell activation marker.

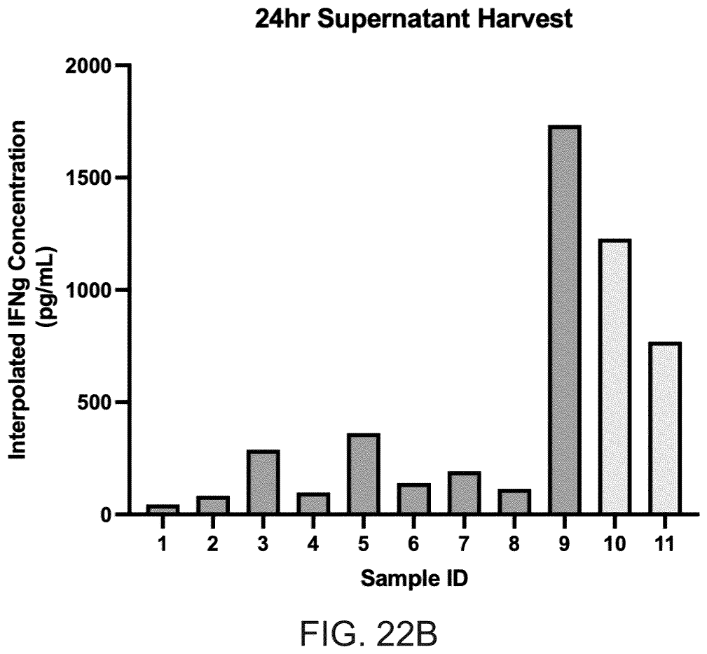

As shown in FIG. 21, 15 μm diameter sized pores with 4.5% PEG at MW 3550 and 0.4 mg/ml streptavidin-acrylamide was conjugated with EpCAM protein at three levels and stained with anti-EpCAM (Alexa Fluor® 405). The results are shown from left to right at low levels of EpCAM, medium levels of EpCAM, and high levels of EpCAM.

### Example 5: Robust Activation of CAR-T Cells with Population of Synthetic Particles Comprising Three Co-Stimulatory Biomolecules (OX40L, 4-1BBL, and the αCD28 Antibody)

Porous hydrogel particles with about 20 Lum average droplet diameter were prepared according to the procedures described in Example 3 above using linear PEG 8000 as an additive in the aqueous phase at 9 weight percent (wt %). These hydrogel particles are referred to as “9% PEG porous hydrogel particles” hereinafter.

Biotinylated proteins (e.g., CD19, 4-1BBL, OX40L, and/or αCD28 antibodies) were then attached to the porous hydrogel particles via biotin-streptavidin interactions. Several different populations of porous hydrogel particles were labeled with two biotinylated proteins, such as:
(1) a population of porous hydrogel particles labeled with CD19 (e.g., with Fc and Avidin tags) and OX40L (with His and Avidin tags);
(2) a population of porous hydrogel particles labeled with CD19 (e.g., with Fc and Avidin tags) and 4-1BBL (with Fc and Avidin tags); and
(3) a population of porous hydrogel particles labeled with CD19 (e.g., with Fc and Avidin tags) and αCD28 antibodies (with His tag).
   The amount of protein bound to bead was maximized to accommodate the number of binding sites.

These different populations of porous hydrogel particles were then combined at about 1:1 ratio prior to culturing with cells. For example, a total of 1e6 particles comprising all three porous hydrogel particles populations (1)-(3) contained about 333,333 particles from each population.

Different combinations of porous hydrogel particles were tested for their ability to stimulate CAR-T cells by co-culturing with commercially available CD19scFv-4-1BB-CD3z CAR-T cell obtained from ProMab Biotechnologies. Briefly, about 1e6 particles of each condition were incubated with about 1e5 commercially available CAR-T cells, as shown in Table 8 below. Thus, the ratio of cells to particles is 1:10. Supernatants were pulled from the culture at different time points, and the amount of secreted IFNg in the supernatants was quantified using the BD CBA IFNg Capture assay. A Higher level of secreted IFNg indicates stronger activation of CAR-T cells.

TABLE 8
Co-Culture Conditions of CAR-T Cells - Porous Hydrogel Particles

| Sample | |
|---|---|
| ID | Co-Culture Conditions |
| 1 | CD19 CAR-T cells without porous particles |
| 2 | CD19 CAR-T cells + Unconjugated porous particles |
| 3 | CD19 CAR-T cells + CD19 labeled porous particles |
| 4 | CD19 CAR-T cells + CD19 labeled porous particles |
| 5 | CD19 CAR-T cells + CD19/OX40L + CD19/4-1BBL |
| | labeled porous particles |
| 6 | CD19 CAR-T cells + CD19/αCD28 labeled porous |
| | particles |
| 7 | CD19 CAR-T cells + CD19/4-1BBL labeled porous |
| | particles |
| 8 | CD19 CAR-T cells + CD19/0X40L labeled porous |
| | particles |
| 9 | CD19 CAR-T cells + CD19/4-1BBL labeled porous |
| | particles + CD19/OX40L labeled porous particles + |
| | CD19/aCD28 labeled porous particles |
| 10 | CD19 CAR-T cells + Jeko-1 mantle cell lymphoma |
| | (MCL) cells |
| 11 | CD19 CAR-T cells + Raji B cells |

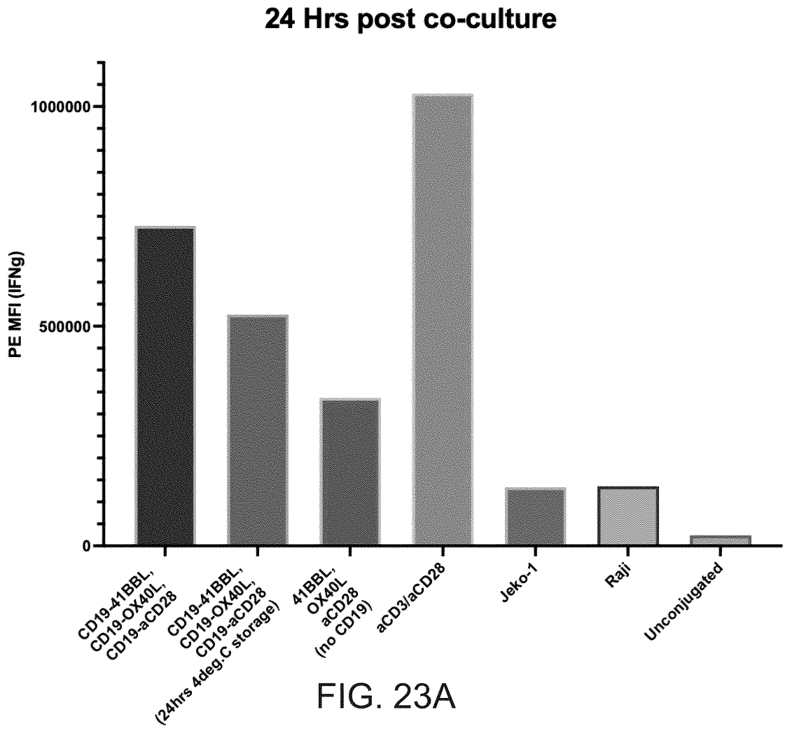

The levels of secreted IFNg at 12-hour or 24-hour post-coculture time points are shown in FIG. 22A and FIG. 22B, respectively. The results indicate that CAR-T cells stimulation is strongest with synthetic particle populations comprising all three co-stimulatory molecules (OX40L, 4-1BBL, and the αCD28 antibody). Indeed, this combination of synthetic beads resulted in stimulation of CAR-T cells at a level that matches or even surpasses that achieved by true biological B-cells (e.g., Jeko-1 or Raji cells), as measured by the IFNg secretion level.

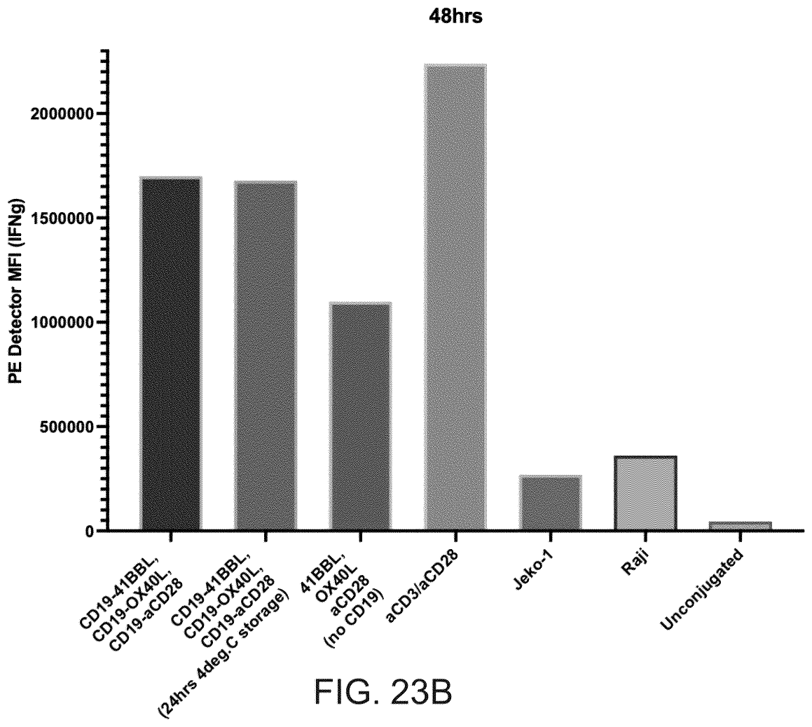

Similar experiments were performed to test the optimal storage conditions of the porous hydrogel particles by monitoring the IFNg secretion level at 24-hour and 48-hour time points post-coculture with CD19scFv-4-1BB-CD3z CAR-T cells from ProMab Biotechnologies. About 1e5 CAR-T cells were co-cultured with about 1e6 porous particles of each condition as indicated in FIG. 23A and FIG. 23B. The results not only confirm that all three co-stimulatory molecules (OX40L, 4-1BBL, and the αCD28 antibody) produce the high CAR-T cell activation, but also demonstrate that these protein-labeled porous hydrogel particles can outperform biological cells for CAR-T cells stimulation. Further, storing the protein-labeled porous hydrogel particles at 4° C. for 24 hours does not significantly reduce their efficacy.

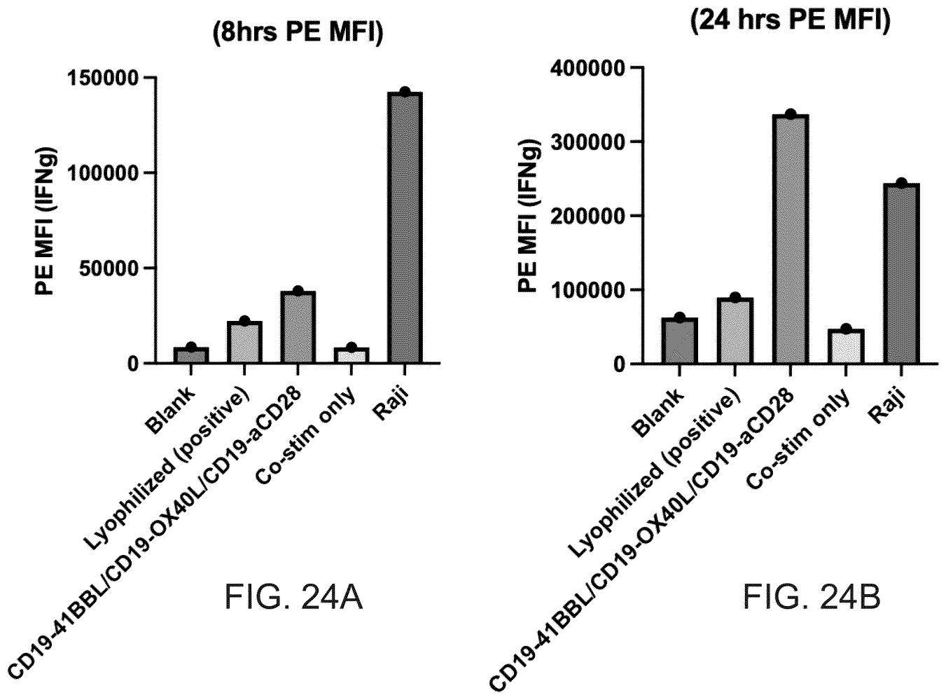

Additional experiments were performed to test the optimal storage conditions of the porous hydrogel particles by monitoring the IFNg secretion level at 8-hour and 24-hour time points post-coculture with CD19scFv-4-1BB-CD3z CAR-T cells from ProMab Biotechnologies. About 1e5 CAR-T cells were co-cultured with about 1e6 porous particles of each condition as indicated in FIG. 24A and FIG. 24B. The results show that the porous hydrogel particles population comprising all three co-stimulatory molecules (OX40L, 4-1BBL, and the αCD28 antibody) displayed only moderate CAR-T cell stimulation at 8-hour post-coculture but robust CAR-T cell stimulation at 24-hour post-coculture, indicating that CAR-T cells stimulation by porous hydrogel particles may require more time than stimulation by biological cells.

### Example 6: Porous Particles Made by Biodegradable Materials can Robustly Induce Cell Response

Size-tunable microsphere were made from an oil/water emulsion of biotin-poly lactic-co-glycolic acid (PLGA)-biotin and 1% polyvinyl alcohol. The microsphere was then streptavidin coated and attached to biotinylated versions of αCD3 and αCD28 antibodies.

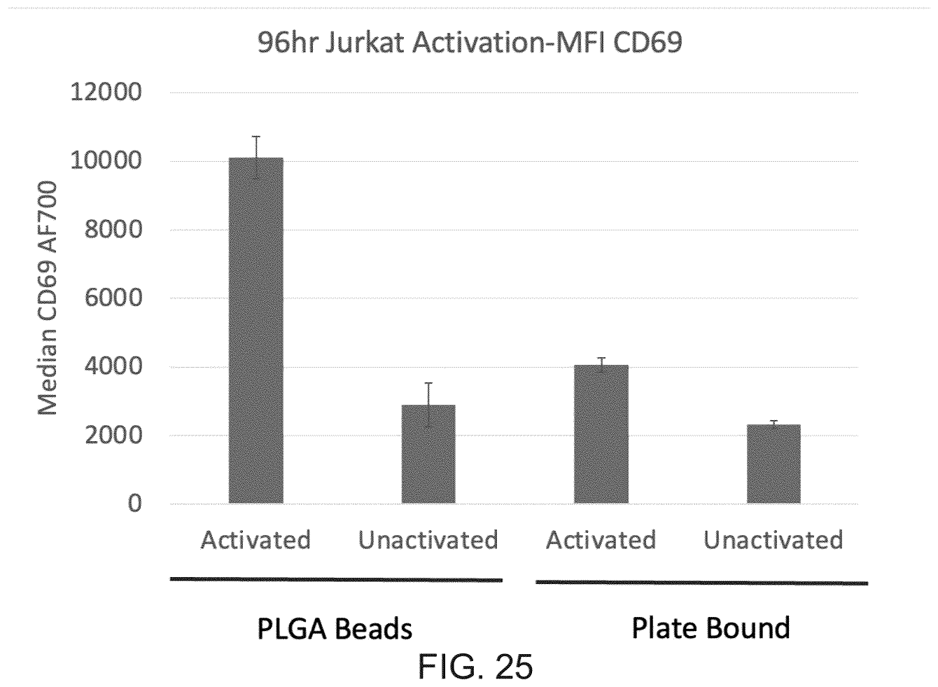

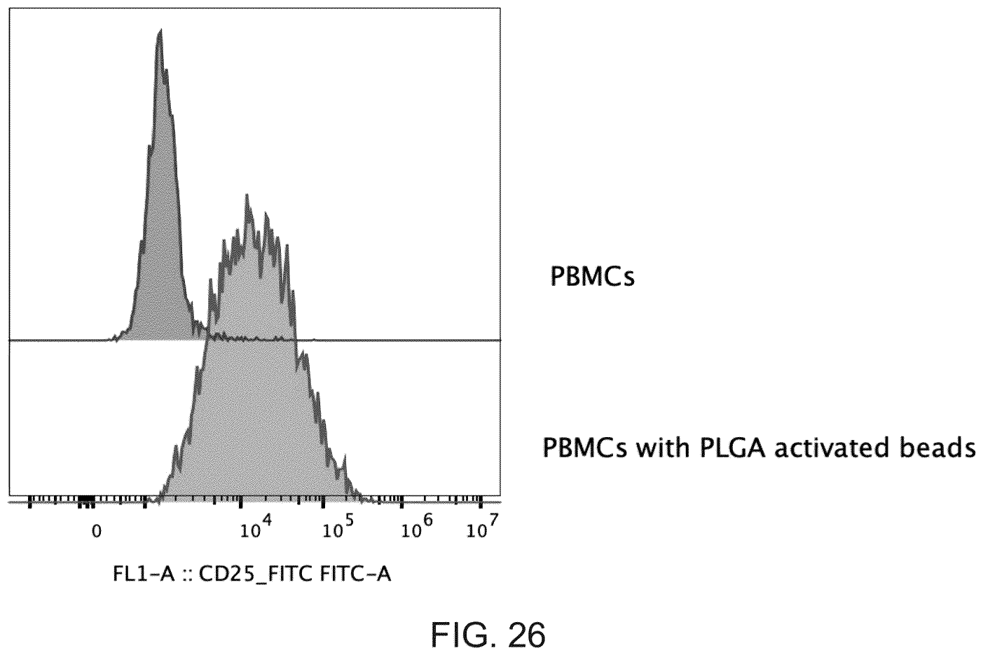

To evaluate the particles in comparison with conventional particles (e.g., Dynabeads), cells (e.g., Jurkat cells) were incubated with the particles at 37° C. After a duration of incubation (e.g., 24 hours of incubation), early-stage cell response (e.g., activation) was assessed by measuring the expression of a marker (e.g., CD69), as shown in FIG. 25. After another duration of incubation (e.g., another 24 or 96 hours of incubation or 48 or 120 hours of incubation total), late-stage cell response was assessed by measuring the expression of a marker (e.g., CD25) by flow cytometry, as shown in FIG. 26. MFI is mean fluorescence intensity. The PLGA-based particles increased both early-stage cell response and late-stage cell response. This reflects sustained activation (e.g., 96 post co-culture for PEG, 300 hours for PLGA).

### Example 7: Porous Particles can Serve as Robust Stimulators of T Cells

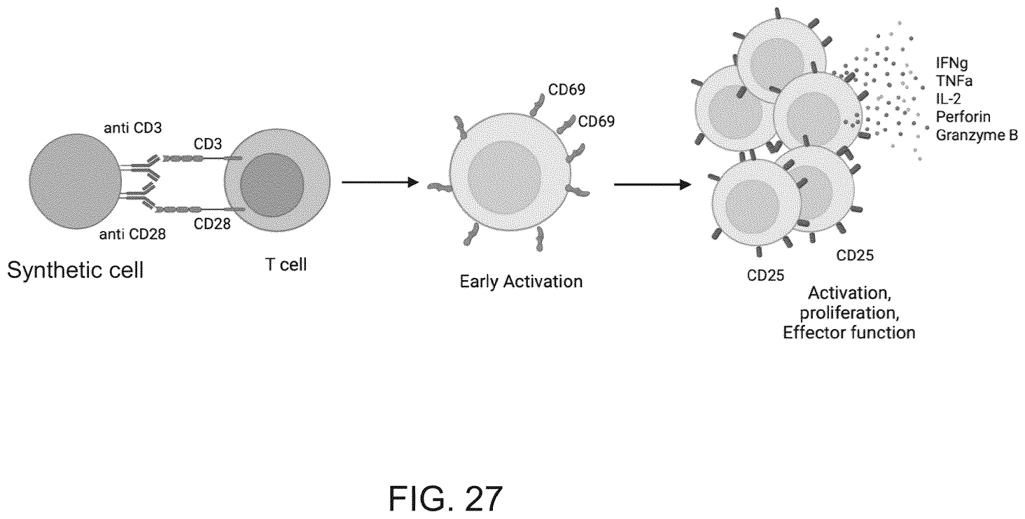

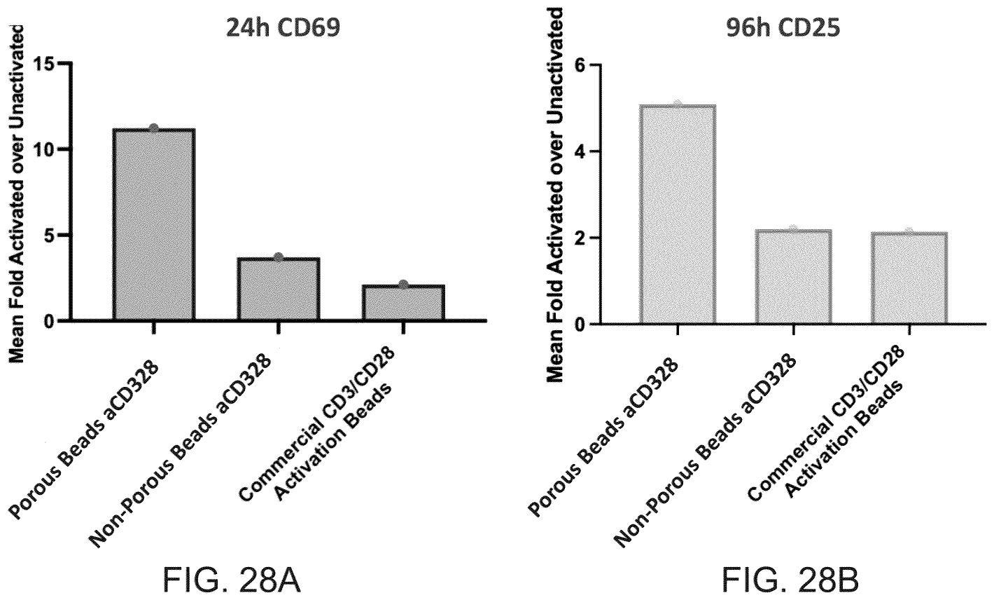

The ability of porous beads and non-porous PLGA beads to stimulate Jurkat T cells was tested alongside a commercial bead product in a co-culture of beads with Jurkat T cells (FIG. 27). Both types of beads were conjugated with equivalent amounts of anti-CD3 and anti-CD28 antibodies. Activation of Jurkat T cells was assessed by measuring, by flow cytometry, upregulation of the cell surface activation marker CD69 at 24-hour timepoint (FIG. 28A) and CD25 at 96-hour timepoint (FIG. 28B) following co-culture. The results show that the porous beads significantly outperform non-porous beads in terms of the ability to activate T-cells.

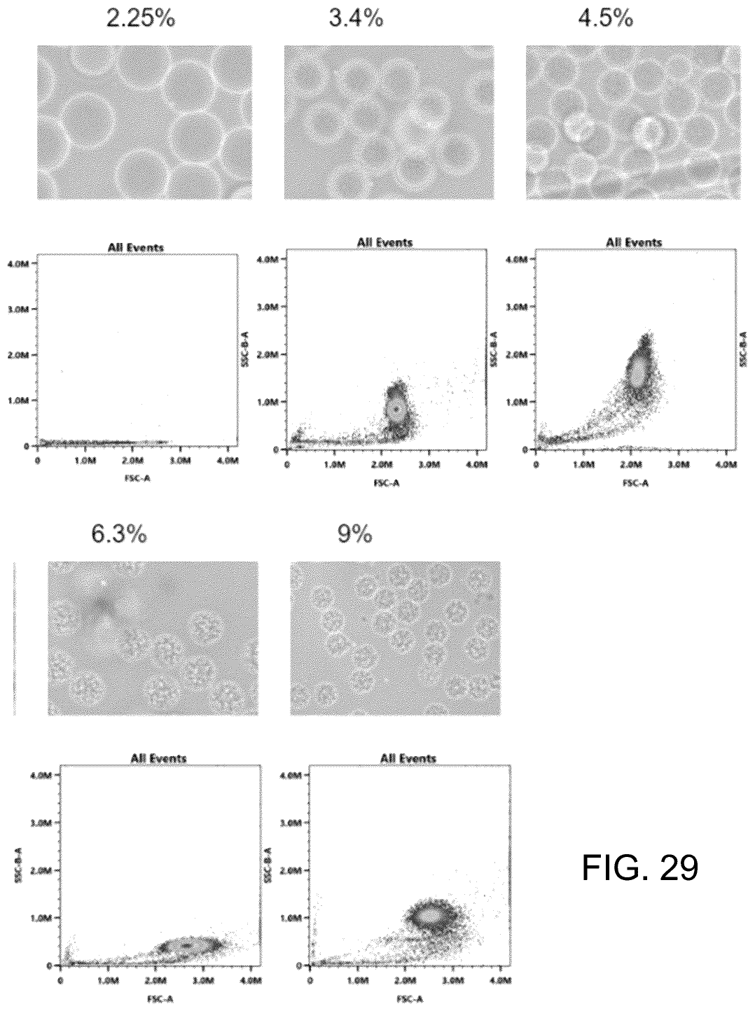

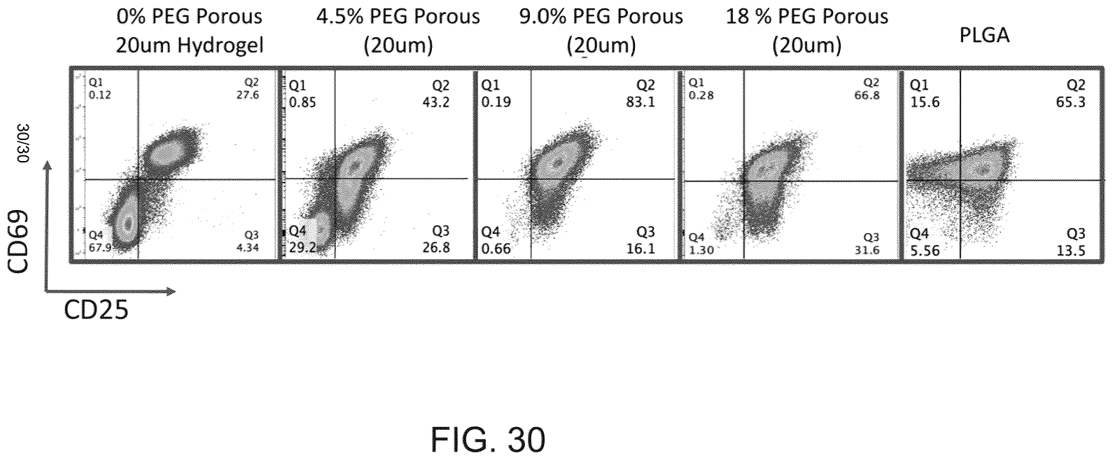

In another set of experiments, the cell activating ability of beads with varying levels of porosity were tested (FIGS. 29 and 30). Different 20 μm beads with varying levels of porosity were prepared using PEG as an additive in the aqueous phase at different weight percentages (wt %), including 2.25%, 3.4%, 4.5%, 6.3%, 9%, and 18%. The beads were conjugated with anti-CD3 and anti-CD28 antibodies and then tested for their ability to stimulate PBMC following co-culture. Activation of PBMC was assessed by measuring upregulation of the cell surface activation markers, CD25 and CD69, by flow cytometry at 48-hour and 72-hour following co-culture. This experiment was performed three times; FIG. 30 shows flow cytometry data from a 72-hour timepoint derived from one experiment, and similar trends were observed at the 48-hour timepoint. Among all the beads tested, the 9% PEG porous beads (i.e., beads prepared with 9% w/v PEG in the aqueous phase) displayed the best capability for immune cell activation, resulting in the highest percentage (83.1%) of cells that expressed both CD69 and CD25 at high levels. The comparison conducted between porous and non-porous particles is expected to produce similar results with other immune co-stimulatory biomolecules or immune response biomolecules. Indeed, other combinations of immune molecules have exhibited surprising results with porous hydrogels. This example will be repeated using other biomolecule combinations disclosed in the instant specification. The results are expected to show superior activation for porous particles.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

### FURTHER NUMBERED EMBODIMENTS

Notwithstanding the appended claims, the disclosure sets forth the following numbered embodiments:

Embodiment 1. A synthetic particle comprising a matrix and at least one immune co-stimulatory biomolecule selected from the group consisting of: (i) a biomolecule that activates 4-1BB receptor signaling; (ii) a biomolecule that activates OX40 receptor signaling; (iii) a biomolecule that activates CD28 receptor signaling; and (iv) any combination thereof.

Embodiment 2. A synthetic particle comprising a matrix and at least one immune co-stimulatory biomolecule selected from the group consisting of a biomolecule that activates the signaling of CD3, a biomolecule that activates the signaling of CD28, a biomolecule that activates the signaling of ICOS (CD278), a biomolecule that activates the signaling of CD27 (TNFRSF7), a biomolecule that activates the signaling of CD40, a biomolecule that activates the signaling of CD40L, a biomolecule that activates the signaling of OX40 (CD134), a biomolecule that activates the signaling of 4-1BB (CD137), a biomolecule that activates the signaling of Toll-like receptor (TLR), a biomolecule that activates the signaling of HVEM (TNFSFR14 or CD270), a biomolecule that activates the signaling of LIGHT (TNFSF14, CD258), a biomolecule that activates the signaling of DR3 (TNFRSF25), a biomolecule that activates the signaling of GITR (CD357), a biomolecule that activates the signaling of CD30 (TNFRSF8), a biomolecule that activates the signaling of TIM1 (HAVCR1, KIM1), a biomolecule that activates the signaling of SLAM (CD150, SLAMF1), a biomolecule that activates the signaling of CD2 (LFA2, OX34), a biomolecule that activates the signaling of CD226 (DNAM1), and any combination thereof.

Embodiment 3. A synthetic biomolecule presenting particle, comprising a matrix and at least one immune co-stimulatory biomolecule selected from the group consisting of: (i) a biomolecule that activates 4-1BB receptor signaling; (ii) a biomolecule that activates OX40 receptor signaling; (iii) a biomolecule that activates CD28 receptor signaling; and (iv) any combination thereof.

Embodiment 4. A synthetic biomolecule presenting particle, comprising a matrix and at least one immune co-stimulatory biomolecule selected from the group consisting of a biomolecule that activates the signaling of CD3, a biomolecule that activates the signaling of CD28, a biomolecule that activates the signaling of ICOS (CD278), a biomolecule that activates the signaling of CD27 (TNFRSF7), a biomolecule that activates the signaling of CD40, a biomolecule that activates the signaling of CD40L, a biomolecule that activates the signaling of OX40 (CD134), a biomolecule that activates the signaling of 4-1BB (CD137), a biomolecule that activates the signaling of Toll-like receptor (TLR), a biomolecule that activates the signaling of HVEM (TNFSFR14 or CD270), a biomolecule that activates the signaling of LIGHT (TNFSF14, CD258), a biomolecule that activates the signaling of DR3 (TNFRSF25), a biomolecule that activates the signaling of GITR (CD357), a biomolecule that activates the signaling of CD30 (TNFRSF8), a biomolecule that activates the signaling of TIM1 (HAVCR1, KIM1), a biomolecule that activates the signaling of SLAM (CD150, SLAMF1), a biomolecule that activates the signaling of CD2 (LFA2, OX34), a biomolecule that activates the signaling of CD226 (DNAM1), and any combination thereof.

Embodiment 5. A synthetic particle comprising a matrix and at least one immune response biomolecule selected from the group consisting of
(i) a 4-1BB receptor;
(ii) an OX40 receptor;
(iii) a CD28 receptor; and
(iv) any combination thereof.

Embodiment 6. A synthetic particle comprising a matrix and at least one immune response biomolecule selected from the group consisting of CD3, CD28, ICOS (CD278), CD27 (TNFRSF7), CD40, CD40L, OX40 (CD134), 4-1BB (CD137), Toll-like receptor (TLR), HVEM (TNFSFR14 or CD270), LIGHT (TNFSF14, CD258), DR3 (TNFRSF25), GITR (CD357), CD30 (TNFRSF8), TIM1 (HAVCR1, KIM1), SLAM (CD150, SLAMF1), CD2 (LFA2, OX34), CD226 (DNAM1), and any combination thereof.

Embodiment 7. The synthetic particle of Embodiment 5 or 6, wherein the immune response biomolecule is attached to the matrix via a linker; optionally, the immune response biomolecule is non-covalently attached to the linker.

Embodiment 8. The synthetic particle of any one of Embodiments 5-7, wherein the immune response biomolecule is tethered to an immune cell.

Embodiment 9. The synthetic particle of any one of Embodiments 5-8, wherein the immune response biomolecule is attached to the matrix via the extracellular portion of the corresponding 4-1BB receptor, the OX40 receptor, and/or the CD28 receptor.

Embodiment 10. The synthetic particle of any one of Embodiments 5-9, wherein:
(i) the 4-1BB receptor is the human 4-1BB receptor;
(ii) the OX40 receptor is the human OX40 receptor; and/or
(iii) the CD28 receptor is the human CD28 receptor.

Embodiment 11. The synthetic particle of any one of Embodiments 5-10, wherein:
(i) the 4-1BB receptor comprises a sequence at least 90%, at least 95%, at least 96%, at least 97%, at least 98%, at least 99%, or 100% identical to amino acids 24-255 of SEQ ID NO: 3;
(ii) the OX40 receptor comprises a sequence at least 90%, at least 95%, at least 96%, at least 97%, at least 98%, at least 99%, or 100% identical to amino acids 30-277 of SEQ ID NO: 4; and/or
(iii) the CD28 receptor comprises a sequence at least 90%, at least 95%, at least 96%, at least 97%, at least 98%, at least 99%, or 100% identical to amino acids 28-220 of SEQ ID NO: 5.

Embodiment 12. The synthetic particle of any one of Embodiments 9-11, wherein:
(i) the extracellular portion of the 4-1BB receptor comprises a sequence at least 90%, at least 95%, at least 96%, at least 97%, at least 98%, at least 99%, or 100% identical to amino acids 24-159 of SEQ ID NO: 3;
(ii) the extracellular portion of the OX40 receptor comprises a sequence at least 90%, at least 95%, at least 96%, at least 97%, at least 98%, at least 99%, or 100% identical to amino acids 30-167 of SEQ ID NO: 4; and/or
(iii) the extracellular portion of the CD28 receptor comprises a sequence at least 90%, at least 95%, at least 96%, at least 97%, at least 98%, at least 99%, or 100% identical to amino acids 28-137 of SEQ ID NO: 5.

Embodiment 13. The synthetic particle according to any one of Embodiments 1-12, comprising at least two of the biomolecules selected from the group consisting of (i)-(iii).

Embodiment 14. The synthetic particle according to any one of Embodiments 1-13, comprising all three biomolecules selected from the group consisting of (i)-(iii).

Embodiment 15. The synthetic particle according to any one of Embodiments 1-14, wherein the synthetic particle comprises an antigen for an immune cell.

Embodiment 16. The synthetic particle according to Embodiment 15, wherein the antigen is CD19.

Embodiment 17. The synthetic particle according to any one of Embodiments 1-16, comprising a cell conjugated to the synthetic particle via the 4-1BB receptor, the OX40 receptor, and/or the CD28 receptor bound to the cell.

Embodiment 18. A population of synthetic particles, said population comprising synthetic particles selected from the group consisting of:
(a) synthetic particles comprising a biomolecule that activates 4-1BB receptor signaling;
(b) synthetic particles comprising a biomolecule that activates OX40 receptor signaling;
(c) synthetic particles comprising a biomolecule that activates CD28 receptor signaling; and
(d) any combination thereof;
   wherein each of the synthetic particles comprises a polymer matrix.

Embodiment 19. A population of synthetic particles, said population comprising synthetic particles selected from the group consisting of:
(a) synthetic particles comprising a 4-1BB receptor immune response biomolecule;
(b) synthetic particles comprising an OX40 receptor immune response biomolecule;
(c) synthetic particles comprising a CD28 receptor immune response biomolecule; and
(d) any combination thereof;
   wherein each of the synthetic particles comprises a polymer matrix.

Embodiment 20. The population of synthetic particles of Embodiment 19, wherein the immune response biomolecule is attached to the matrix via a linker; optionally, the immune response biomolecule is non-covalently attached to the linker.

Embodiment 21. The population of synthetic particles of any one of Embodiments 19-20, wherein the immune response biomolecule is tethered to an immune cell.

Embodiment 22. The population of synthetic particles of any one of Embodiments 19-21, wherein the immune response biomolecule is attached to the matrix via the extracellular portion of the corresponding 4-1BB receptor; OX40 receptor, and/or the CD28 receptor.

Embodiment 23. The population of synthetic particles of any one of Embodiments 19-22, wherein:
(a) the 4-1BB receptor is the human 4-1BB receptor;
(b) the OX40 receptor is the human OX40 receptor; and/or
(c) the CD28 receptor is the human CD28 receptor.

Embodiment 24. The population of synthetic particles of any one of Embodiments 19-23, wherein:
(a) the 4-1BB receptor comprises a sequence at least 90%, at least 95%, at least 96%, at least 97%, at least 98%, at least 99%, or 100% identical to amino acids 24-255 of SEQ ID NO: 3;
(b) the OX40 receptor comprises a sequence at least 90%, at least 95%, at least 96%, at least 97%, at least 98%, at least 99%, or 100% identical to amino acids 30-277 of SEQ ID NO: 4; and/or
(c) the CD28 receptor comprises a sequence at least 90%, at least 95%, at least 96%, at least 97%, at least 98%, at least 99%, or 100% identical to amino acids 28-220 of SEQ ID NO: 5.

Embodiment 25. The population of synthetic particles of Embodiment 22-24, wherein:
(a) the extracellular portion of the 4-1BB receptor comprises a sequence at least 90%, at least 95%, at least 96%, at least 97%, at least 98%, at least 99%, or 100% identical to amino acids 24-159 of SEQ ID NO: 3;
(b) the extracellular portion of the OX40 receptor comprises a sequence at least 90%, at least 95%, at least 96%, at least 97%, at least 98%, at least 99%, or 100% identical to amino acids 30-167 of SEQ ID NO: 4; and/or
(c) the extracellular portion of the CD28 receptor comprises a sequence at least 90%, at least 95%, at least 96%, at least 97%, at least 98%, at least 99%, or 100% identical to amino acids 28-137 of SEQ ID NO: 5.

Embodiment 26. The population of synthetic particles according to any one of Embodiments 18-25, wherein the population comprises (a).

Embodiment 27. The population of synthetic particles according to any one of Embodiments 18-25, wherein the population comprises (b).

Embodiment 28. The population of synthetic particles according to any one of Embodiments 18-25, wherein the population comprises (c).

Embodiment 29. The population of synthetic particles according to any one of Embodiments 18-25, wherein the population comprises (a) and (b).

Embodiment 30. The population of synthetic particles according to any one of Embodiments 18-25, wherein the population comprises (a) and (c).

Embodiment 31. The population of synthetic particles according to any one of Embodiments 18-25, wherein the population comprises (b) and (c).

Embodiment 32. The population of synthetic particles according to any one of Embodiments 18-25, wherein the population comprises (a), (b), and (c).

Embodiment 33. The population of synthetic particles according to any one of Embodiments 18-32, wherein (a), (b), and (c) are distinct synthetic particles.

Embodiment 34. The population of synthetic particles according to any one of Embodiments 18-32, wherein (a), (b) are the same synthetic particles that are distinct from (c).

Embodiment 35. The population of synthetic particles according to any one of Embodiments 18-32, wherein (a), (c) are the same synthetic particles that are distinct from (b).

Embodiment 36. The population of synthetic particles according to any one of Embodiments 18-32, wherein (b), (c) are the same synthetic particles that are distinct from (a).

Embodiment 37. The population of synthetic particles according to any one of Embodiments 18-32, wherein (a), (b), and (c) are the same synthetic particles.

Embodiment 38. The population of synthetic particles according to any one of Embodiments 18-37, wherein at least one of the synthetic particles comprises a cell conjugated to the synthetic particle via a 4-1BB receptor, an OX40 receptor, and/or a CD28 receptor expressed by the cell.

Embodiment 39. A population of synthetic particles comprising one or more synthetic particles according to any one of Embodiments 1-17.

Embodiment 40. The population of synthetic particles of Embodiment 39, wherein the population comprises one or more different subpopulations, each subpopulation according to any one of Embodiments 1-17.

Embodiment 41. The population of synthetic particles according to any one of Embodiments 18-40, wherein the molar ratio of the biomolecule that activates 4-1BB receptor signaling to the biomolecule that activates OX40 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

Embodiment 42. The population of synthetic particles according to any one of Embodiments 18-41, wherein the molar ratio of the biomolecule that activates 4-1BB receptor signaling to the biomolecule that activates CD28 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

Embodiment 43. The population of synthetic particles according to any one of Embodiments 18-42, wherein the molar ratio of the biomolecule that activates OX40 receptor signaling to the biomolecule that activates CD28 receptor signaling is between about 1:100 and about 1:10, between about 1:10 and about 1:3, between about 1:3 and about 1:1, between about 2:1 and about 1:2, between about 1:1 and about 1:3, between about 1:3 and about 1:10, or between about 1:10 and about 1:100.

Embodiment 44. The population of synthetic particles according to any one of Embodiments 18-43, wherein at least one synthetic particle comprises an antigen for an immune cell.

Embodiment 45. The population of synthetic particles according to Embodiment 44, wherein the antigen is CD19.

Embodiment 46. A mixture of (i) cells and (ii) the population of synthetic particles according to any one of Embodiments 18-45.

Embodiment 47. The mixture of Embodiment 46, wherein the mixture is essentially free of feeder cells.

Embodiment 48. A cell-particle conjugate, wherein the cell-particle conjugate comprises a cell and the synthetic particle of any of Embodiments 1-17.

Embodiment 49. A cell-particle conjugate, wherein the cell-particle conjugate comprises a cell and the population of synthetic particles according to any one of Embodiments 18-45.

Embodiment 50. A cell, wherein the cell is conjugated to the synthetic particle of any of Embodiments 1-17.

Embodiment 51. A cell, wherein the cell is conjugated to the population of synthetic particles according to any one of Embodiments 18-45.

Embodiment 52. The mixture of Embodiment 46 or 47, the cell-particle conjugate of Embodiment 48 or 49, or the cell of Embodiment 50 or 51, wherein the cell and the particle(s) are non-covalently conjugated.

Embodiment 53. The mixture of Embodiment 46 or 47, the cell-particle conjugate of Embodiment 48 or 49, or the cell of Embodiment 50 or 51, or the mixture, the cell-particle conjugate, or the cell of Embodiment 52, wherein the cell expresses at least one of 4-1BB receptor, OX40 receptor, and CD28 receptor; optionally, wherein the cell expresses at least two of 4-1BB receptor, OX40 receptor, and CD28 receptor; optionally, wherein the cell expresses 4-1BB receptor, OX40 receptor, and CD28 receptor.

Embodiment 54. The mixture, the cell-particle conjugate, or the cell of Embodiment 53, wherein the conjugation between the cell and the particle(s) comprises an interaction between at least one of (i) 4-1BB receptor and the biomolecule that activates 4-1BB receptor signaling, (ii) OX40 receptor and the biomolecule that activates OX40 receptor signaling, and (iii) CD28 receptor and the biomolecule that activates CD28 receptor signaling; optionally, the conjugation comprises interactions between at least two of (i)-(iii); optionally, the conjugation comprises interactions between all of (i)-(iii).

Embodiment 55. The mixture of Embodiment 46 or 47, the cell-particle conjugate of Embodiment 48 or 49, or the cell of Embodiment 50 or 51, or the mixture, the cell-particle conjugate, or the cell of any of Embodiments 52-54, wherein the cell is an immune cell.

Embodiment 56. The synthetic particle of any one of Embodiments 8-17, the population of synthetic particles according to any one of Embodiments 21-45, the mixture of Embodiment 46 or 47, the cell-particle conjugate of Embodiment 48 or 49, or the cell of Embodiment 50 or 51, or the mixture, the cell-particle conjugate, or the cell of any of Embodiments 52-55, wherein the immune cell is a T cell; optionally, the immune cell is a cytotoxic T cell; optionally, the immune cell is a CAR-T cell.

Embodiment 57. The synthetic particle, the population of synthetic particles, the mixture, the cell-particle conjugate, or the cell of Embodiment 56, wherein the antigen binds to a chimeric antigen receptor (CAR) expressed by the immune cell.

Embodiment 58. The synthetic particle of any of Embodiments 1-17 and 56-57, the population of synthetic particles of any of Embodiments 18-45 and 56-57, the mixture of any of Embodiments 46-47 and 52-57, the cell-particle conjugate of any of Embodiments 48-49 and 52-57, or the cell of any one of Embodiments 50-57, wherein the biomolecule that activates 4-1BB receptor signaling comprises an anti-4-1BB receptor antibody or antigen binding fragment thereof, or comprises a 4-1BB ligand (4-1BBL) or a functional fragment thereof.

Embodiment 59. The synthetic particle, the population of synthetic particles, the mixture, the cell-particle conjugate, or the cell of Embodiment 58, wherein the 4-1BBL or the functional fragment thereof comprises an amino acid sequence at least 80%, at least 85%, at least 90%, at least 95%, at least 96%, at least 97%, at least 98%, at least 99%, or 100% identical to amino acids 80-244, or amino acids 50-254 of SEQ ID NO: 1.

Embodiment 60. The synthetic particle, the population of synthetic particles, the mixture, the cell-particle conjugate, or the cell of Embodiment 58 or 59, wherein the 4-1BBL or the functional fragment thereof is capable of activating the signaling of 4-1BB receptor expressed on a surface of an immune cell.

Embodiment 61. The synthetic particle of any of Embodiments 1-17 and 56-60, the population of synthetic particles of any of Embodiments 18-45 and 56-60, the mixture of any of Embodiments 46-47 and 52-60, the cell-particle conjugate of any of Embodiments 48-49 and 52-60, or the cell of any one of Embodiments 50-60, wherein the biomolecule that activates OX40 receptor signaling comprises an anti-OX40 receptor antibody or antigen binding fragment thereof, or comprises an OX40 ligand (OX40L) or a functional fragment thereof.

Embodiment 62. The synthetic particle, the population of synthetic particles, the mixture, the cell-particle conjugate, or the cell of Embodiment 61, wherein the OX40L or the functional fragment thereof comprises an amino acid sequence at least 80%, at least 85%, at least 90%, at least 95%, at least 96%, at least 97%, at least 98%, at least 99%, or 100% identical to amino acids 61-174, or amino acids 51-183 of SEQ ID NO: 2.

Embodiment 63. The synthetic particle, the population of synthetic particles, the mixture, the cell-particle conjugate, or the cell of Embodiment 61 or 62, wherein the OX40L or the functional fragment thereof is capable of activating the signaling of OX40 receptor expressed on a surface of an immune cell.

Embodiment 64. The synthetic particle of any of Embodiments 1-17 and 56-63, the population of synthetic particles of any of Embodiments 18-45 and 56-63, the mixture of any of Embodiments 46-47 and 52-63, the cell-particle conjugate of any of Embodiments 48-49 and 52-63, or the cell of any one of Embodiments 50-63, wherein the biomolecule that activates CD28 receptor signaling comprises an anti-CD28 antibody or antigen binding fragment thereof, a B7-1 (CD80) ligand or a functional fragment thereof, or a B7-2 (CD86) ligand or a functional fragment thereof.

Embodiment 65. The synthetic particle, the population of synthetic particles, the mixture, the cell-particle conjugate, or the cell of Embodiment 64, wherein the biomolecule that activates CD28 receptor signaling comprises an anti-CD28 antibody or antigen binding fragment thereof.

Embodiment 66. The synthetic particle, the population of synthetic particles, the mixture, the cell-particle conjugate, or the cell of Embodiment 65, wherein the anti-CD28 antibody is a mouse IgG1 monoclonal antibody (clone CD28.2) available from BioLegend®.

Embodiment 67. The synthetic particle, the population of synthetic particles, the mixture, the cell-particle conjugate, or the cell of any one of Embodiments 64-66, wherein the biomolecule that activates CD28 receptor signaling binds CD28 receptor with a dissociation constant (Kd) of less than 10 μM, less than 1 μM, less than 100 nM, less than 10 nM, or less than 1 nM.

Embodiment 68. The synthetic particle of any of Embodiments 1-17 and 56-67, the population of synthetic particles of any of Embodiments 18-45 and 56-67, the mixture of any of Embodiments 46-47 and 52-67, the cell-particle conjugate of any of Embodiments 48-49 and 52-67, or the cell of any one of Embodiments 50-67, wherein the synthetic particle(s) further comprise a molecule selected from the group consisting of: a biologic; an antibody or an antigen-binding fragment thereof; an antibody drug conjugate; a protein; an enzyme; a peptide; a non-ribosomal peptide.

Embodiment 69. The synthetic particle of any of Embodiments 1-17 and 56-68, the population of synthetic particles of any of Embodiments 18-45 and 56-68, the mixture of any of Embodiments 46-47 and 52-68, the cell-particle conjugate of any of Embodiments 48-49 and 52-68, or the cell of any one of Embodiments 50-68, wherein the synthetic particle(s) further comprise a molecule selected from CD3; CD4; CD8; CD19; CD14; ccr7; CD45; CD45RA; CD27; CD16; CD56; CD127; CD25; CD38; HLA-DR; PD-1; CD28; CD183; CD185; CD57; IFN-gamma; CD20; TCR gamma/delta; TNF alpha; CD69; IL-2; Ki-67; CCR6; CD34; CD45RO; CD161; IgD; CD95; CD117; CD123; CD11c; IgM; CD39; FoxP3; CD10; CD40L; CD62L; CD194; CD314; IgG; TCR V alpha 7.2; CD11b; CD21; CD24; IL-4; Biotin; CCR10; CD31; CD44; CD138; CD294; NKp46; TCR V delta 2; TIGIT; CD1c; CD2; CD7; CD8a; CD15; CD32; CD103; CD107a; CD141; CD158; CD159c; IL-13; IL-21; KLRG1; TIM-3; CCR5; CD5; CD33; CD45.2; CD80; CD159a (NKG2a); CD244; CD272; CD278; CD337; Granzyme B; Ig Lambda Light Chain; IgA; IL-17A; Streptavidin; TCR V delta 1; CD1d; CD26; CD45R (B220); CD64; CD73; CD86; CD94; CD137; CD163; CD193; CTLA-4; CX3CR1; Fe epsilon R1 alpha; IL-22; Lag-3; MIP-1 beta; Perforin; TCR V gamma 9; CD1a; CD22; CD36; CD40; CD45R; CD66b; CD85j; CD160; CD172a; CD186; CD226; CD303; CLEC12A; CXCR4; Helios; Ig Kappa Light Chain; IgE; IgG1; IgG3; IL-5; IL-8; IL-21 R; KIR3dl05; KLRC1/2; Ly-6C; Ly-6G; MHC Class II (I-A/I-E); MHC II; TCR alpha/beta; TCR beta; TCR V alpha 24; Akt (pS473); ALDH1A1; Annexin V; Bcl-2; c-Met; CCR7; cd16/32; cd41a; CD3 epsilon; CD8b; CD11b/c; CD16/CD32; CD23; CD29; CD43; CD45.1; CD48; CD49b; CD49d; CD66; CD68; CD71; CD85k; CD93; CD99; CD106; CD122; CD133; CD134; CD146; CD150; CD158b; CD158b1/b2; CD158e; CD166; CD169; CD184; CD200; CD200 R; CD235a; CD267; CD268; CD273; CD274; CD317; CD324; CD326; CD328; CD336; CD357; CD366; DDR2; eFluor 780 Fix Viability; EGF Receptor; EGFR (pY845); EOMES; EphA2; ERK1/2 (pT202/pY204); F4/80; FCRL5; Flt-3; FVS575V; FVS700; Granzyme A; HER2/ErbB2; Hes1; Hoechst (33342); ICAM-1; IFN-alpha; IgAQ1; IgA1/IgA2; IgA2; IgG2; IgG4; IL-1 RAcP; IL-6; IL-10; IL-12; IL-17; Integrin alpha 4 beta 7; Isotype Ctrl; KLRC1; KLRC2; Live/Dead Fix Aqua; Ly-6A/Ly-6E; Ly-6G/Ly-6C; Mannose Receptor; MDR1; Met (pY1234/pY1235); MMP-9; NGF Receptor p75; ORAI1; ORAI2; ORAI3; p53; P2RY12; PARP; cleaved; RT1B; S6 (pS235/pS236); STIM1; STIM2; TCR delta; TCR delta/gamma; TCR V alpha 24 J alpha 18; TCR V beta 11; TCR V gamma 1.1; TCR V gamma 2; TER-119; TIMP-3; TRAF3; TSLP Receptor; VDAC1; Vimentin; XCR1; and YAP1.

Embodiment 70. The synthetic particle of any of Embodiments 1-17 and 56-69, the population of synthetic particles of any of Embodiments 18-45 and 56-69, the mixture of any of Embodiments 46-47 and 52-69, the cell-particle conjugate of any of Embodiments 48-49 and 52-69, or the cell of any one of Embodiments 50-69, wherein the synthetic particle(s) do not contain a CD3 binding molecule.

Embodiment 71. The synthetic particle of any of Embodiments 1-17 and 56-70, the population of synthetic particles of any of Embodiments 18-45 and 56-70, the mixture of any of Embodiments 46-47 and 52-70, the cell-particle conjugate of any of Embodiments 48-49 and 52-70, or the cell of any one of Embodiments 50-70, wherein the synthetic particle(s) do not contain a CD8 binding molecule.

Embodiment 72. The synthetic particle of any of Embodiments 1-17 and 56-71, the population of synthetic particles of any of Embodiments 18-45 and 56-71, the mixture of any of Embodiments 46-47 and 52-71, the cell-particle conjugate of any of Embodiments 48-49 and 52-71, or the cell of any one of Embodiments 50-71, wherein the synthetic particle(s) further comprise at least one T cell stimulatory molecule and/or at least one T cell co-stimulatory molecule.

Embodiment 73. The synthetic particle of any of Embodiments 1-17 and 56-72, the population of synthetic particles of any of Embodiments 18-45 and 56-72, the mixture of any of Embodiments 46-47 and 52-72, the cell-particle conjugate of any of Embodiments 48-49 and 52-72, or the cell of any one of Embodiments 50-72, wherein the biomolecule is biotinylated.

Embodiment 74. The synthetic particle of any of Embodiments 1-17 and 56-73, the population of synthetic particles of any of Embodiments 18-45 and 56-73, the mixture of any of Embodiments 46-47 and 52-73, the cell-particle conjugate of any of Embodiments 48-49 and 52-73, or the cell of any one of Embodiments 50-73, wherein at least one surface of the matrix is functionalized; optionally, wherein the functionalized surface comprises a linker.

Embodiment 75. The synthetic particle, the population of synthetic particles, the mixture, the cell-particle conjugate, or the cell of Embodiment 74, wherein the functionalization comprises conjugating, coating, and/or embedding the linker to and/or within the matrix.

Embodiment 76. The synthetic particle of any of Embodiments 1-17 and 56-75, the population of synthetic particles of any of Embodiments 18-45 and 56-75, the mixture of any of Embodiments 46-47 and 52-75, the cell-particle conjugate of any of Embodiments 48-49 and 52-75, or the cell of any one of Embodiments 50-75, wherein the biomolecule is bound to the matrix via a linker; optionally, wherein the linker comprises streptavidin.

Embodiment 77. The synthetic particle of any of Embodiments 1-17 and 56-76, the population of synthetic particles of any of Embodiments 18-45 and 56-76, the mixture of any of Embodiments 46-47 and 52-76, the cell-particle conjugate of any of Embodiments 48-49 and 52-76, or the cell of any one of Embodiments 50-76, wherein the biomolecule is non-covalently or covalently bound to the matrix.

Embodiment 78. The synthetic particle of any of Embodiments 1-17 and 56-77, the population of synthetic particles of any of Embodiments 18-45 and 56-77, the mixture of any of Embodiments 46-47 and 52-77, the cell-particle conjugate of any of Embodiments 48-49 and 52-77, or the cell of any one of Embodiments 50-77, wherein the matrix is a substantially spherical matrix.

Embodiment 79. The synthetic particle of any of Embodiments 1-17 and 56-78, the population of synthetic particles of any of Embodiments 18-45 and 56-78, the mixture of any of Embodiments 46-47 and 52-78, the cell-particle conjugate of any of Embodiments 48-49 and 52-78, or the cell of any one of Embodiments 50-78, wherein the matrix comprises a polymer material derived from one or more monomers.

Embodiment 80. The synthetic particle, the population of synthetic particles, the mixture, the cell-particle conjugate, or the cell of Embodiment 79, wherein the one or more monomers are selected from group consisting of: hydroxyethyl methacrylate, ethyl methacrylate, 2-hydroxyethyl methacrylate (HEMA), propylene glycol methacrylate, N-vinylpyrrolidone (NVP), methyl methacrylate, glycidyl methacrylate, glycerol methacrylate (GMA), glycol methacrylate, lactic acid, glycolic acid, poly(lactic-co-glycolic) acid (PLGA), ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzyl acrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, N-phenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, acrylamide, bisacrylamide, streptavidin-acrylamide, N-vinylcarbazole, 4-vinylpyridine, 2-vinylpyridine, agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitin, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan.

Embodiment 81. The synthetic particle of any of Embodiments 1-17 and 56-80, the population of synthetic particles of any of Embodiments 18-45 and 56-80, the mixture of any of Embodiments 46-47 and 52-80, the cell-particle conjugate of any of Embodiments 48-49 and 52-80, or the cell of any one of Embodiments 50-80, wherein the matrix is biodegradable.

Embodiment 82. The synthetic particle, the population of synthetic particles, the mixture, the cell-particle conjugate, or the cell of any one of Embodiments 79-81, wherein the one or more monomers comprise a monosaccharide, disaccharide, polysaccharide, peptide, protein, or protein domain.

Embodiment 83. The synthetic particle, the population of synthetic particles, the mixture, the cell-particle conjugate, or the cell of any one of Embodiments 79-82, wherein the one or more monomers comprise a protein or protein domain comprising at least one non-natural amino acid.

Embodiment 84. The synthetic particle, the population of synthetic particles, the mixture, the cell-particle conjugate, or the cell of any one of Embodiments 79-83, wherein the one or more monomers comprise a structural polysaccharide.

Embodiment 85. The synthetic particle, the population of synthetic particles, the mixture, the cell-particle conjugate, or the cell of any one of Embodiments 79-84, wherein the one or more monomers are selected from the group consisting of agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitin, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan.

Embodiment 86. The synthetic particle of any of Embodiments 1-17 and 56-85, the population of synthetic particles of any of Embodiments 18-45 and 56-85, the mixture of any of Embodiments 46-47 and 52-85, the cell-particle conjugate of any of Embodiments 48-49 and 52-85, or the cell of any one of Embodiments 50-85, wherein the polymer material comprises poly(lactic-co-glycolic acid) (PLGA).

Embodiment 87. The synthetic particle, the population of synthetic particles, the mixture, the cell-particle conjugate, or the cell of Embodiment 86, wherein the PLGA has a composition of poly(lactic acid):poly(glycolic acid) of between about 90:10 and about 10:90.

Embodiment 88. The synthetic particle, the population of synthetic particles, the mixture, the cell-particle conjugate, or the cell of Embodiment 79-87, wherein at least 50%, at least 60%, at least 70%, at least 80%, at least 90%, at least 95%, at least 98%, or at least 99% of the matrix is the polymer material derived from the one or more monomers.

Embodiment 89. The synthetic particle of any of Embodiments 1-17 and 56-88, the population of synthetic particles of any of Embodiments 18-45 and 56-88, the mixture of any of Embodiments 46-47 and 52-88, the cell-particle conjugate of any of Embodiments 48-49 and 52-88, or the cell of any one of Embodiments 50-88, wherein the synthetic particle(s) further comprise at least one fluorophore.

Embodiment 90. The synthetic particle of any of Embodiments 1-17 and 56-89, the population of synthetic particles of any of Embodiments 18-45 and 56-89, the mixture of any of Embodiments 46-47 and 52-89, the cell-particle conjugate of any of Embodiments 48-49 and 52-89, or the cell of any one of Embodiments 50-89, wherein the synthetic particle(s) have a (mean) diameter of between about 1 μm and about 40 μm, between about 10 μm and about 30 μm, between about 15 μm and about 25 μm, or about 20 μm.

Embodiment 91. The synthetic particle of any of Embodiments 1-17 and 56-90, the population of synthetic particles of any of Embodiments 18-45 and 56-90, the mixture of any of Embodiments 46-47 and 52-90, the cell-particle conjugate of any of Embodiments 48-49 and 52-90, or the cell of any one of Embodiments 50-90, wherein the synthetic particle(s) are hydrogel particles.

Embodiment 92. The synthetic particle of any of Embodiments 1-17 and 56-91, the population of synthetic particles of any of Embodiments 18-45 and 56-91, the mixture of any of Embodiments 46-47 and 52-91, the cell-particle conjugate of any of Embodiments 48-49 and 52-91, or the cell of any one of Embodiments 50-91, wherein the synthetic particle(s) have a (mean) porosity of about 5% to about 95% of a volume of the synthetic particle(s); optionally, the synthetic particle(s) have a (mean) porosity of between about 80% and about 95% of the volume of the synthetic particle(s).

Embodiment 93. The synthetic particle of any of Embodiments 1-17 and 56-92, the population of synthetic particles of any of Embodiments 18-45 and 56-92, the mixture of any of Embodiments 46-47 and 52-92, the cell-particle conjugate of any of Embodiments 48-49 and 52-92, or the cell of any one of Embodiments 50-92, wherein the synthetic particle(s) comprise a plurality of micropores and a plurality of macropores within the matrix.

Embodiment 94. The synthetic particle, the population of synthetic particles, the mixture, the cell-particle conjugate, or the cell of Embodiment 93, wherein the mean diameter of the plurality of macropores is between about 200 nm and about 2 μm.

Embodiment 95. The synthetic particle, the population of synthetic particles, the mixture, the cell-particle conjugate, or the cell of Embodiment 93 or 94, wherein the synthetic particle comprises the plurality of macropores at a concentration of at least 2.25% v/v, at least 3.4% v/v, and/or at least 4.5% v/v.

Embodiment 96. The synthetic particle, the population of synthetic particles, the mixture, the cell-particle conjugate, or the cell of Embodiments 93-95, wherein the mean diameter of the plurality of micropores is between about 1 nm and about 20 nm; optionally, between about 2 nm and about 4 nm.

Embodiment 97. The synthetic particle, the population of synthetic particles, the mixture, the cell-particle conjugate, or the cell of Embodiments 93-96, wherein the plurality of macropores comprise between about 2% and about 30% of a total number of pores of the synthetic particle, the total number of pores of the synthetic particle being a combination of the plurality of micropores and the plurality of macropores.

Embodiment 98. The synthetic particle of any of Embodiments 1-17 and 56-97, the population of synthetic particles of any of Embodiments 18-45 and 56-97, the mixture of any of Embodiments 46-47 and 52-97, the cell-particle conjugate of any of Embodiments 48-49 and 52-97, or the cell of any one of Embodiments 50-97, wherein the synthetic particle(s) exhibit a (mean) Young's modulus of between about 0.2 kPa and about 400 kPa.

Embodiment 99. The synthetic particle of any of Embodiments 1-17 and 56-98, the population of synthetic particles of any of Embodiments 18-45 and 56-98, the mixture of any of Embodiments 46-47 and 52-98, the cell-particle conjugate of any of Embodiments 48-49 and 52-98, or the cell of any one of Embodiments 50-98, wherein the biomolecule is located on a surface of the particle(s).

Embodiment 100. The synthetic particle, the population of synthetic particles, the mixture, the cell-particle conjugate, or the cell of Embodiment 99, wherein the surface of the particle is an internal surface or an external surface.

Embodiment 101. The synthetic particle, the population of synthetic particles, the mixture, the cell-particle conjugate, or the cell of Embodiment 100, wherein the internal surface is within the plurality of macropores.

Embodiment 102. A method of inducing proliferation, expansion, and/or activation of immune cells in culture, comprising contacting or culturing the immune cells with the synthetic particle of any of Embodiments 1-17 and 56-101 or the population of synthetic particles of any of Embodiments 18-45 and 56-101.

Embodiment 103. A method of inducing an immune cell response, comprising contacting or culturing the immune cell with the synthetic particle of any of Embodiments 1-17 and 56-101 or the population of synthetic particles of any of Embodiments 18-45 and 56-101.

Embodiment 104. The method of Embodiment 103, wherein the immune cell response includes activation and/or expansion of the immune cell.

Embodiment 105. The method of Embodiment 103 or 104, wherein the immune cell response is determined by (i) IL-2 secretion from the immune cell; (ii) CD25 expression from the immune cell; or (iii) CD69 expression from the immune cell.

Embodiment 106. The method of Embodiment 103 or 104, wherein the immune cell response is determined by interferon-gamma (IFNg) secretion from the immune cell.

Embodiment 107. The method of any one of Embodiments 103-106, wherein the immune cell response from contacting the immune cell with the synthetic particle(s) is at least 50%, at least 100%, at least 2-fold, at least 3-fold, at least 5-fold, or at least 10-fold higher than the immune cell response from a control immune cell contacted with otherwise identical synthetic particle(s) lacking the biomolecule or macropores.

Embodiment 108. The method of any one of Embodiments 102-107, wherein contacting comprises exposing the immune cells to the synthetic particles at a ratio of immune cell:synthetic particle of between about 1:0.5 and about 1:50, between about 1:1 and about 1:40, between about 1:2 and about 1:30, between about 1:5 and about 1:20, or about 1:10.

Embodiment 109. The method of any one of Embodiments 102-108, wherein the contacting or culturing of the immune cell with the synthetic particle(s) lasts more than 8 hours.

Embodiment 110. A method of treating a disease or disorder in a subject in need thereof, comprising administering the activated immune cells obtained by the method according to any one of Embodiments 102-109 to the subject.

Embodiment 111. A method of treating a disease or disorder in a subject in need thereof, comprising administering the synthetic particle of any of Embodiments 1-17 and 56-101, the population of synthetic particles of any of Embodiments 18-45 and 56-101, the mixture of any of Embodiments 46-47 and 52-101, the cell-particle conjugate of any of Embodiments 48-49 and 52-101, or the cell of any one of Embodiments 50-101, to the subject.

Embodiment 112. The method of Embodiment 110 or 111, wherein the disease or disorder is a cancer, an autoimmune disease, or an infectious disease.

Embodiment 113. A method of preparing the synthetic particle of any of Embodiments 1-17 and 56-101, comprising: preparing a precursor particle comprising the matrix and attaching the biomolecule to the precursor particle.

Embodiment 114. The method of Embodiment 113, wherein the method comprises attaching the antigen for the immune cell to the precursor particle.

Embodiment 115. A method of preparing or the population of synthetic particles of any of Embodiments 18-45 and 56-101, comprising: (i) preparing precursor particles comprising the matrix; (ii) attaching the biomolecules to the precursor particles.

Embodiment 116. The method of Embodiment 115, wherein step (ii) comprises attaching the two or more groups of biomolecule groups (i)-(iii) to separate precursor particles and then mixing the precursor particles.

Embodiment 117. The method of any of Embodiments 113-116, comprising attaching the antigen for the immune cell to at least part of the precursor particle.

Embodiment 118. The method of any of Embodiments 113-117, wherein preparing the precursor particle(s) comprises:
mixing a base material with a porogen;
forming microspheres from the mixture;
thermally curing the microspheres; and
washing the microspheres to remove the porogen,
wherein the base material comprises a monomer and a linker.

Embodiment 119. The method of any of Embodiments 113-117, wherein preparing the precursor particle(s) comprises:
mixing a first phase comprising a monomer and porogens, with a second phase, wherein the first phase and the second phase are immiscible;
polymerizing the first phase, thereby encapsulating or embedding porogens within the polymerized monomer;
removing the porogens from the polymerized monomer to form the precursor particle(s).

Embodiment 120. The method of Embodiment 119, wherein the first phase is an aqueous phase and the second phase is a non-aqueous phase.

Embodiment 121. The method of Embodiment 119 or 120, wherein the first phase is a dispersed phase and the second phase is a continuous phase.

1.-121. (canceled)

122. A population of hydrogel particles, comprising:
a combination of immune response biomolecules, comprising a 4-1BB receptor, an OX40 receptor, and a CD28 receptor;
wherein each hydrogel particle of the population of hydrogel particles comprises a polymer matrix, and an immune response biomolecule of the combination of immune response biomolecules is attached to the polymer matrix.

123. The population of hydrogel particles of claim 122, wherein the immune response biomolecules of the combination of immune response molecules are immune co-stimulatory biomolecules.

124. The population of hydrogel particles of claim 122, wherein:
(a) the 4-1BB receptor is human 4-1BB receptor;
(b) the OX40 receptor is human OX40 receptor; or
(c) the CD28 receptor is human CD28 receptor.

125. The population of hydrogel particles of claim 122, wherein:
(a) the 4-1BB receptor comprises a sequence at least 90%, identical to amino acids 24-255 of SEQ ID NO: 3;
(b) the OX40 receptor comprises a sequence at least 90% identical to amino acids 30-277 of SEQ ID NO: 4; or
(c) the CD28 receptor comprises a sequence at least 90% identical to amino acids 28-220 of SEQ ID NO: 5.

126. The population of hydrogel particles of claim 122, wherein the immune response biomolecule is attached to the polymer matrix via an extracellular portion of the corresponding 4-1BB receptor; OX40 receptor, or the CD28 receptor.

127. The population of hydrogel particles of claim 126, wherein:
(a) the extracellular portion of the 4-1BB receptor comprises a sequence at least 90%, identical to amino acids 24-159 of SEQ ID NO: 3;
(b) the extracellular portion of the OX40 receptor comprises a sequence at least 90%, identical to amino acids 30-167 of SEQ ID NO: 4; or
(c) the extracellular portion of the CD28 receptor comprises a sequence at least 90%, identical to amino acids 28-137 of SEQ ID NO: 5.

128. The population of hydrogel particles of claim 122, wherein each hydrogel particle of the population of hydrogel particles further comprises an antigen for an immune cell.

129. The population of hydrogel particles of claim 128, wherein the antigen is CD19.

130. The population of hydrogel particles of claim 122, further comprising a cell conjugated to at least one hydrogel particle of the plurality of hydrogel particles via at least one of the 4-1BB receptor, the OX40 receptor, or the CD28 receptor.

131. The population of hydrogel particles of claim 122, comprising a plurality of sub-populations of hydrogel particles, wherein each sub-population comprises a distinct immune response biomolecule from the combination of immune response biomolecules attached to the corresponding hydrogel particles of the sub-population.

132. The population of hydrogel particles of claim 131, wherein each sub-population further comprises CD19 attached to the corresponding hydrogel particles of the sub-population.

133. The population of hydrogel particles of claim 131, comprising:
a first sub-population of hydrogel particles comprising the 4-1BB receptor;
a second sub-population of hydrogel particles comprising the OX40 receptor; and
a third sub-population of hydrogel particles comprising the CD28 receptor.

134. The population of hydrogel particles of claim 133, wherein
the first sub-population of hydrogel particles comprises CD19 and the 4-1BB receptor;
the second sub-population of hydrogel particles comprises CD19 and the OX40 receptor; and
the third sub-population of hydrogel particles comprises CD19 and the CD28 receptor.

135. The population of hydrogel particles of claim 122, comprising a plurality of sub-populations of hydrogel particles, wherein at least one sub-population comprises two or more immune response biomolecule from the combination of immune response biomolecules attached to the corresponding hydrogel particles of the sub-population.

136. The population of hydrogel particles of claim 135, wherein the at least one sub-population comprises the 4-1BB receptor and the OX40 receptor, the 4-1BB receptor and the CD28 receptor, the OX40 receptor and the CD 28 receptor, or the 4-1BB receptor, the OX40 receptor and the CD28 receptor.

137. The population of hydrogel particles of claim 135, wherein the at least one sub-population further comprises CD19.

138. A method of inducing an immune cell response, comprising contacting or culturing a plurality of immune cells with the population of hydrogel particles of claim 122.

139. The method of claim 138, wherein the immune cell response comprises one or both of activation and expansion of the plurality of immune cells.

140. A method of treating a disease or disorder in a subject in need thereof, comprising administering a plurality of activated immune cells obtained by contacting or culturing a plurality of immune cells with the population of hydrogel particles of claim 122.

141. The method of claim 140, wherein the disease or disorder is a cancer, an autoimmune disease, or an infectious disease.


SEQUENCE LISTING (38 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 4 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 5 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 7 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 8 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 9 - protein, 163 aa, Homo sapiens
ACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCG

SEQ ID NO: 10 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 12 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 209 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLT

SEQ ID NO: 14 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 15 - protein, 138 aa, Homo sapiens
DVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 16 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 17 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 18 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 19 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 21 - protein, 151 aa, Homo sapiens
HEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 22 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 23 - protein, 121 aa, Homo sapiens
CKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLC

SEQ ID NO: 24 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 25 - protein, 161 aa, Homo sapiens
DGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 26 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 27 - protein, 122 aa, Homo sapiens
EPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 28 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 29 - protein, 153 aa, Homo sapiens
CSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 30 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 31 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 32 - protein, 195 aa, Homo sapiens
MMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFS

SEQ ID NO: 33 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 34 - protein, 184 aa, Homo sapiens
HLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLS

SEQ ID NO: 35 - protein, 92 aa, Homo sapiens
SQQIYGVVYGNVTFHVPSNVPLKEVLWKKQKDKVAELENSEFRAFSSFKNRVYLDTVSGSLTIYNLTSSDEDEYEMESPNITDTMKFFLYVL

SEQ ID NO: 36 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 37 - protein, 305 aa, Homo sapiens
PGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQ

SEQ ID NO: 38 - protein, 314 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIF